(19) United States
(12) Patent Application Publication
(10) Pub. No.: US 2023/0339866 A1
(43) Pub. Date: Oct. 26, 2023

(54) Quinazoline compounds and the use thereof in the treatment of cancer

(71) Applicant: TROBIO THERAPEUTICS PTY LTD, Beecroft, New South Wales (AU)

(72) Inventors: Edna HARDEMAN, Beecroft, New South Wales (AU); Peter GUNNING, Castle Hill, New South Wales (AU); Eleanor EIFFE, Newtown, New South Wales (AU)

(21) Appl. No.: 17/795,515

(22) PCT Filed: Feb. 1, 2021
(86) PCT No.: PCT/AU2021/050074

§ 371 Date: Jul. 26, 2022

(30) Foreign Application Priority Data
Feb. 4, 2020 (AU) 2020900302
Jun. 18, 2020 (AU) 2020902029

(51) Int. Cl. C07D 239/95; C07D 409/04; C07D 403/04; C07D 405/04; C07D 403/12; C07D 409/14; C07D 403/14; C07D 405/12; C07D 401/12; C07D 409/12; A61P 35/00

(52) U.S. Cl. CPC C07D 239/95; C07D 409/04; C07D 403/04; C07D 405/04; C07D 403/12; C07D 409/14; C07D 403/14; C07D 405/12; C07D 401/12; C07D 409/12; A61P 35/00

(57) ABSTRACT

The present disclosure relates generally to a class of quinazoline compounds, compositions containing the same and the therapeutic use of the compounds in the treatment of cancer.

### FIELD OF THE DISCLOSURE

The present disclosure relates generally to a class of quinazoline compounds, compositions containing the same and the therapeutic use of the compounds in the treatment of cancer.

### BACKGROUND OF THE DISCLOSURE

Cancer continues to kill many thousands of people annually throughout the world and is fast becoming a global pandemic. The global cancer burden is expected to increase to 21.7 million cases and 13 million deaths worldwide by 2030. There is therefore an ever present need to develop new and improved cancer therapies in an effort to arrest cancer burden on society.

Against this background the present inventors have developed a new class of quinazoline compounds that show promise in the treatment of a number of cancers.

### SUMMARY OF THE DISCLOSURE

In a first aspect there is provided a compound having the following formula (I):

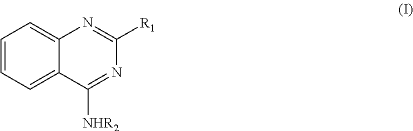

or a pharmaceutically acceptable salt, hydrate, derivative, solvate or prodrug thereof, wherein:
R₁ is selected from the group consisting of:
(i) a heteroaryl group having between 5 and 14 ring atoms in which one or more of the ring atoms are selected from nitrogen, sulfur and oxygen, a heterocyclyl group having between 5 and 14 ring atoms in which one or more of the ring atoms are selected from nitrogen and oxygen,
(ii)
(iii)

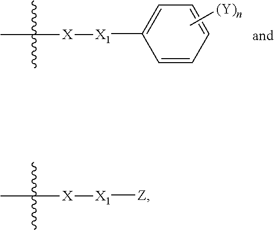

wherein X is O, S or NH;
X₁ is absent or is a straight-chain or branched-chain alkanediyl group having between 1 and 6 carbon atoms;
Y is selected from the group consisting of: CN, NR₅R₆, OH, C₁-C₆alkoxy, halo, CF₃ and C₁-C₆ alkyl;
Z is selected from the group consisting of:a heteroaryl group having 5 or 6 ring atoms in which one or more of the ring atoms are nitrogen, oxygen or sulfur, and (methylenedioxy)phenyl, and wherein the heteroaryl group is optionally substituted with a methyl group;
n is 0, 1, 2 or 3;
R₅ and R₆ are independently selected from the group consisting of: H and C₁-C₆ alkyl;
R₂ is selected from the group consisting of:
(i) T-heteroaryl, wherein the heteroaryl group has 5 ring atoms in which one or more of the ring atoms are nitrogen;
(ii) T-OH
(iii) T-OCH₃
(iv) T-NH₂ and,
(v)

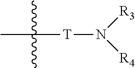

wherein T is a straight-chain or branched-chain alkanediyl group having between 1 and 10 carbon atoms;
R₃ and R₄ are independently selected from the group consisting of: H and C₁—C alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group.

In a second aspect there is provided a pharmaceutical composition comprising a compound of formula (I) according to the first aspect together with a pharmaceutically acceptable carrier, diluent or excipient.

The composition may further comprise a vinca alkaloid or a taxane.

The vinca alkaloid may be vinorelbine and the taxane may be paclitaxel.

The composition may be a synergistic composition.

In a third aspect there is provided a method for the treatment of cancer in a subject in need thereof, the method comprising administration to the subject of a therapeutically effective amount of a compound of formula (I) according to the first aspect or a composition of the second aspect.

The method may further comprise administration of a vinca alkaloid or a taxane.

The vinca alkaloid may be vinorelbine and the taxane may be paclitaxel.

The cancer may be neuroblastoma, ovarian cancer or lung cancer.

In a fourth aspect there is provided use of a compound of formula (I) according to the first aspect in the manufacture of medicament for the treatment of cancer.

The medicament may further comprise, or may be administered with, a vinca alkaloid or a taxane.

The vinca alkaloid may be vinorelbine and the taxane may be paclitaxel.

In a fifth aspect there is provided a compound of formula (I) according to the first aspect for use in the treatment of cancer.

In a sixth aspect there is provided a method for reducing incidences of, or risk of, cancer recurrence in a subject deemed to be at risk of cancer recurrence, the method comprising administration to the subject of an effective amount of a compound of formula (I) according to the first aspect, or a composition of the second aspect.

In a seventh aspect there is provided use of a compound of formula (I) according to the first aspect in the manufacture of a medicament for reducing incidences of, or risk of, cancer recurrence in a subject deemed to be at risk of cancer recurrence.

In an eighth aspect there is provided a compound of formula (I) according to the first aspect for use in reducing incidences of, or risk of, cancer recurrence in a subject deemed to be at risk of cancer recurrence.

### BRIEF DESCRIPTION OF THE FIGURES

FIG. 1: The interactions of compounds 11, 13, 32 and 92 with the N-terminus and overlap region of Tpm3.1 measured using circular dichroism spectroscopy

FIG. 2: Major thermal transitions from individual isotherms. Data were further smoothed by the Savitzky-Golay method (5-point window), differentiated and then fitted to multiple Gaussian peaks.

FIG. 3. Actin depolymerization data for compound 92.

FIG. 4. Imaging and quantitation of actin filaments in SK-N-SH neuroblastoma cells treated with compounds 57, 87, and 92. Cells were transfected with Tpm3.1 tagged with the mCherry fluorophore.

FIG. 5. Imaging and quantitation of actin filaments in SK-N-SH neuroblastoma cells treated with compounds 57, 87 and 92. Cells were stained with 488-Atto-Phalloidin.

### DEFINITIONS

The following are some definitions that may be helpful in understanding the description of the present disclosure. These are intended as general definitions and should in no way limit the scope of the present disclosure to those terms alone, but are put forth for a better understanding of the following description.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The terms “a” and “an” are used herein to refer to one or to more than one (i.e. to at least one) of the grammatical object of the article. By way of example, “an element” means one element or more than one element.

In the context of this specification, the term “alkyl” is taken to mean straight-chain or branched-chain monovalent saturated hydrocarbon groups having the recited number of carbon atoms. Examples of alkyl groups include, but are not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl and the like.

In the context of this specification, the term “alkoxy” is taken to mean O-alkyl groups in which alkyl is as defined herein. Examples of alkoxy groups include, but are not limited to, methoxy, ethoxy, n-propoxy, isopropoxy, sec-butoxy and tert-butoxy.

In the context of this specification, the terms “halo” and “halogen” are used interchangeably and refer to fluorine, chlorine, bromine and iodine.

In the context of this specification, the term “heteroaryl” refers to a monocyclic, bicyclic, or tricyclic aromatic ring system having the recited total number of ring atoms, wherein the ring system contains at least one nitrogen, sulfur or oxygen atom, the remaining ring atoms being carbon. Examples of heteroaryl include, but are not limited to pyridyl, pyrrolyl, indolyl, quinolinyl, furnayl, thienyl, oxazolyl, thiazolyl and the like.

In the context of this specification, the term “heterocyclyl” refers to a non-aromatic, saturated or partially unsaturated ring system having the recited total number of ring atoms, wherein the ring system contains at least one nitrogen, sulfur or oxygen atom, the remaining ring atoms being carbon. The term also includes substituents in which the heterocyclyl group is fused with an aromatic ring. Examples of heterocyclyl include, but are not limited to pyrrolidinyl, piperazinyl, 2,3-dihydroindolyl, piperidinyl, azetidinyl, pyrazolinyl, morpholinyl, dihydroquinolinyl and the like.

In the context of this specification the term “alkanediyl” is understood to refer to a bivalent saturated branched-chain or straight-chain hydrocarbon group conforming to the formula CₙH₂ₙ.

In the context of this specification, the term “prodrug” means a compound which is able to be converted in vivo by metabolic means (e.g. by hydrolysis, reduction or oxidation) to a compound of the formula (I).

In the context of this specification, the term “effective amount” includes a non-toxic but sufficient amount of an active compound to provide the stated effect. When used in reference to cancer recurrence, “effective amount” means an amount of a compound of formula (I) that is required to reduce the incidence of, or risk of an individual experiencing cancer recurrence. Those skilled in the art will appreciate that the exact amount of a compound required will vary based on a number of factors and thus it is not possible to specify an exact “effective amount”. However, for any given case an appropriate “effective amount” may be determined by one of ordinary skill in the art.

In the context of this specification, the term “therapeutically effective amount” includes a non-toxic but sufficient amount of an active compound to provide the desired therapeutic effect. Those skilled in the art will appreciate that the exact amount of a compound required will vary based on a number of factors and thus it is not possible to specify an exact “therapeutically effective amount”. However, for any given case an appropriate “therapeutically effective amount” may be determined by one of ordinary skill in the art.

In the context of this specification, the terms “treating”, “treatment”, “preventing” and “prevention” refer to any and all uses that remedy cancer or symptoms thereof, prevent the establishment of cancer, or otherwise prevent, hinder, retard or reverse the progression of cancer or other undesirable symptoms in any way whatsoever. Thus, the terms “treating”, “treatment”, “preventing” and “prevention” and the like are to be considered in their broadest context. For example, treatment does not necessarily imply that a subject is treated until total recovery.

In the context of this specification, the term “subject” includes human and also non-human animals. As such, in addition to being useful in the treatment of cancer in humans, the compounds of the present disclosure also find use in the treatment of cancer in non-human animals, for example mammals such as companion animals and farm animals. Non-limiting examples of companion animals and farm animals include dogs, cats, horses, cows, sheep and pigs. Preferably, the subject is a human.

In the context of this specification the term “recurrence” as it relates to cancer is understood to mean the return of cancerous cells and/or a cancerous tumour after cancerous cells and/or a cancerous tumour have been successfully treated previously.

In the context of this specification the term “administering” and variations of that term including “administer” and “administration”, includes contacting, applying, delivering or providing a compound or composition to an organism by any appropriate means.

### DETAILED DESCRIPTION

In one aspect of the present disclosure there is provided a compound having the following formula (I):

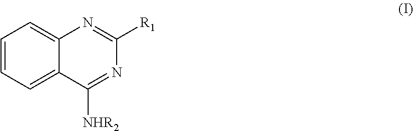

or a pharmaceutically acceptable salt, hydrate, derivative, solvate or prodrug thereof, wherein:
R₁ is selected from the group consisting of:
(i) a heteroaryl group having between 5 and 14 ring atoms in which one or more of the ring atoms are selected from nitrogen, sulfur and oxygen, a heterocyclyl group having between 5 and 14 ring atoms in which one or more of the ring atoms are selected from nitrogen and oxygen,
(ii)
(iii)

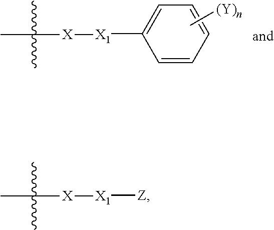

wherein X is O, S or NH;
X, is absent or is a straight-chain or branched-chain alkanediyl group having between 1 and 6 carbon atoms;
Y is selected from the group consisting of: CN, NR₅R₆, OH, C₁-C₆alkoxy, halo, CF₃ and C₁-C₆ alkyl;
Z is selected from the group consisting of:a heteroaryl group having 5 or 6 ring atoms in which one or more of the ring atoms are nitrogen, oxygen or sulfur, and (methylenedioxy)phenyl, and wherein the heteroaryl group is optionally substituted with a methyl group;
n is 0, 1, 2 or 3;
R₅ and R₆ are independently selected from the group consisting of: H and C₁-C₆ alkyl;
R₂ is selected from the group consisting of:
(i) T-heteroaryl, wherein the heteroaryl group has 5 ring atoms in which one or more of the ring atoms are nitrogen;
(ii) T-OH
(iii) T-OCH₃
(iv) T-NH₂ and,
(v)

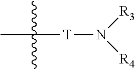

wherein T is a straight-chain or branched-chain alkanediyl group having between 1 and 10 carbon atoms;
R₃ and R₄ are independently selected from the group consisting of: H and C₁-C₆ alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group.

In one embodiment, the heteroaryl group of R₁ may have 5 or 6 ring atoms in which one or more of the ring atoms are selected from nitrogen, sulfur and oxygen, and the heterocyclyl group of R₁ may have 5 or 6 ring atoms in which one or more of the ring atoms are selected from nitrogen and oxygen.

In another embodiment the heteroaryl group of R₁ may have 5 ring atoms in which one or more of the ring atoms are selected from nitrogen, sulfur and oxygen, and the heterocyclyl group of R₁ may have 5 ring atoms in which one or more of the ring atoms are selected from nitrogen and oxygen.

In a further embodiment the heteroaryl group of R₁ may have 5 ring atoms in which one or two of the ring atoms are selected from nitrogen, sulfur and oxygen and the heterocyclyl group of R₁ may have 5 ring atoms in which one or two of the ring atoms are selected from nitrogen and oxygen.

In still another embodiment the heteroaryl group of R₁ may be thienyl, pyrrolyl or furanyl, and the heterocyclic group may be pyrrolidinyl.

In some embodiments X is NH.

In some embodiments X₁ is absent or is a straight-chain or branched-chain alkanediyl group having between 1 and 5 carbon atoms.

In some embodiments X₁ is absent or is a straight-chain or branched-chain alkanediyl group having between 1 and 4 carbon atoms.

In other embodiments X, is absent or is a straight-chain or branched-chain alkanediyl group having between 1 and 3 carbon atoms.

In still further embodiments X₁ is absent or is —CH₂— or —CH₂CH₂—.

In a further embodiment X₁ is —CH₂— or —CH₂CH₂—.

In some embodiments Y is selected from the group consisting of: CN, NR₅R₆, OH, OMe, halo, CF₃ and C₁-C₆ alkyl.

In other embodiments Y is selected from the group consisting of: CN, NR₅R₆, OH, OMe, F, Cl, CF₃ and C₁-C₄ alkyl.

In further embodiments Z is selected from the group consisting of: a heteroaryl group having 5 or 6 ring atoms in which one or two of the ring atoms are nitrogen, oxygen or sulfur, and (methylenedioxy)phenyl, and wherein the heteroaryl group is optionally substituted with a methyl group.

In yet other embodiments Z is selected from the group consisting of: a heteroaryl group having 5 or 6 ring atoms in which one of the ring atoms is nitrogen, oxygen or sulfur, and (methylenedioxy)phenyl, and wherein the heteroaryl group is optionally substituted with a methyl group.

In another embodiment Z is thienyl, pyrrolyl, methylpyrrolyl, furanyl, pyridyl or (methylenedioxy)phenyl.

In a further embodiment n is 0, 1 or 2.

In yet another embodiment n is 0 or 1.

In still a further embodiment n is 1.

In a further embodiment R₅ and R₆ are independently selected from hydrogen and C₁-C₃ alkyl.

In another embodiment R₅ and R₆ are independently selected from hydrogen and methyl.

In one embodiment R₁ is a heteroaryl group having 5 or 6 ring atoms in which one or more of the ring atoms are selected from nitrogen, sulfur and oxygen, or a heterocyclyl group having 5 or 6 ring atoms in which one or more of the ring atoms are selected from nitrogen and oxygen.

In another embodiment R₁ is a heteroaryl group having 5 ring atoms in which one or more of the ring atoms are selected from nitrogen, sulfur and oxygen, or a heterocyclyl group having 5 ring atoms in which one or more of the ring atoms are selected from nitrogen and oxygen.

In still a further embodiment R₁ is a heteroaryl group having 5 ring atoms in which one or two of the ring atoms are selected from nitrogen, sulfur and oxygen or a heterocyclyl group having 5 ring atoms in which one or two of the ring atoms are selected from nitrogen and oxygen.

In a further embodiment R₁ is thienyl, pyrrolyl, furanyl or pyrrolidinyl.

In another embodiment R₁ is

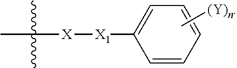

In a further embodiment R₁ is

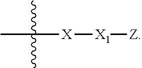

In one embodiment R₁ is a heteroaryl group having 5 or 6 ring atoms in which one or more of the ring atoms are selected from nitrogen, sulfur and oxygen, or a heterocyclyl group having 5 or 6 ring atoms in which one or more of the ring atoms are selected from nitrogen and oxygen, or R₁ is

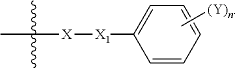

In another embodiment R₁ is a heteroaryl group having 5 ring atoms in which one or more of the ring atoms are selected from nitrogen, sulfur and oxygen, or a heterocyclyl group having 5 ring atoms in which one or more of the ring atoms are selected from nitrogen and oxygen, or R₁ is

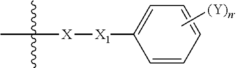

In still a further embodiment R₁ is a heteroaryl group having 5 ring atoms in which one or two of the ring atoms are selected from nitrogen, sulfur and oxygen, or a heterocyclyl group having 5 ring atoms in which one or two of the ring atoms are selected from nitrogen and oxygen, or R₁ is

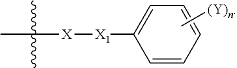

In a further embodiment R₁ is thienyl, pyrrolyl, furanyl, pyrrolidinyl or

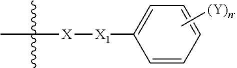

In other embodiments R, is selected from the group consisting of:

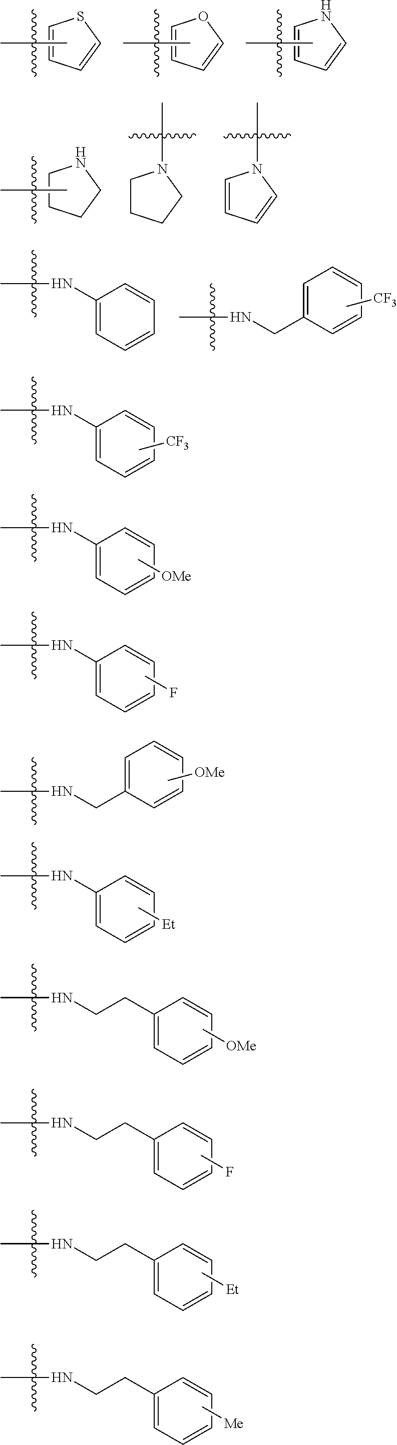

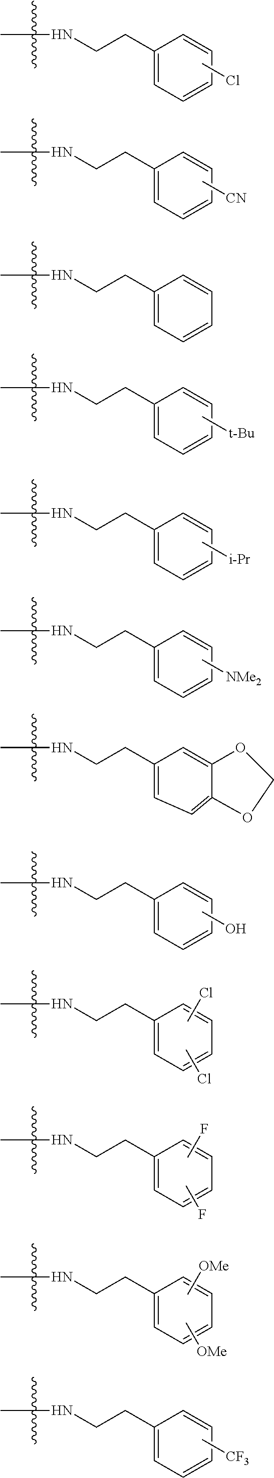

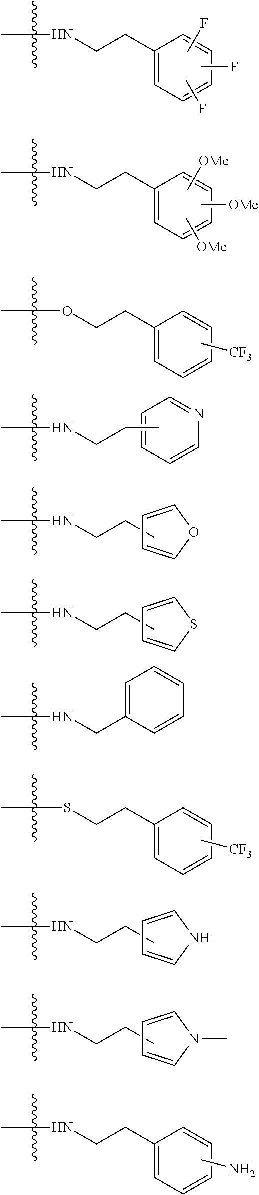

In one embodiment, in the T-heteroaryl substituent, the heteroaryl group has 5 ring atoms in which one or two of the ring atoms are nitrogen.

In one embodiment, in the T-heteroaryl substituent, the heteroaryl group is imidazolyl or pyrrolyl.

In one embodiment, in the T-heteroaryl substituent, the heteroaryl group is imidazolyl.

In some embodiments R₃ and R₄ are independently selected from the group consisting of: H and C₁-C₃ alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group.

In other embodiment R₃ and R₄ are independently selected from the group consisting of: H and C₁-C₃ alkyl, or together with the nitrogen to which they are attached form a saturated 5- or 6-membered ring having 0 or 1 additional nitrogen atoms or 1 oxygen atom, wherein the ring is optionally substituted with a C₁-C₆ alkyl group.

In further embodiments R₃ and R₄ are independently selected from the group consisting of: H and C₁-C₃ alkyl, or together with the nitrogen to which they are attached form a saturated 5- or 6-membered ring having 0 or 1 additional nitrogen atoms or 1 oxygen atom, wherein the ring is optionally substituted with a C₁-C₃ alkyl group.

In yet another embodiment R₃ and R₄ are independently selected from the group consisting of: H and methyl, or together with the nitrogen to which they are attached form a saturated 5- or 6-membered ring having 0 or 1 additional nitrogen atoms or 1 oxygen atom, wherein the ring is optionally substituted with a methyl group.

In a further embodiment R₃ and R₄ are independently selected from the group consisting of: H and methyl, or together with the nitrogen to which they are attached form a saturated 5- or 6-membered ring selected from:

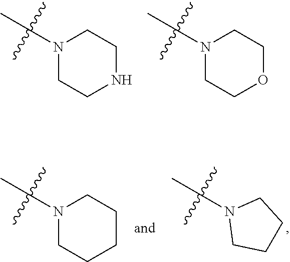 [SMILES: *N1CCNCC1 ; *N1CCOCC1 ; *N1CCCCC1 ; *N1CCCC1]

wherein each ring is optionally substituted with a methyl group.

In still a further embodiment R₃ and R₄ are independently selected from the group consisting of: H and methyl, or together with the nitrogen to which they are attached form a saturated 5- or 6-membered ring selected from:

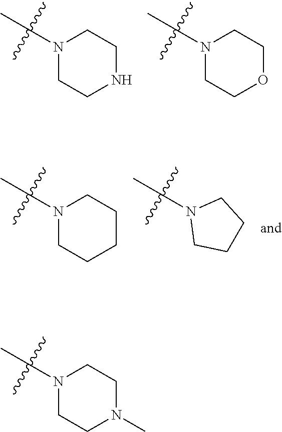

In one embodiment R₂ is T-OH.

In another embodiment R₂ is T-OCH₃.

In a further embodiment R₂ is T-NH₂.

In yet another embodiment R₂ is

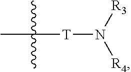

wherein R₃ and R₄ are independently selected from the group consisting of: H and C₁-C₃ alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group.

In a further embodiment R₂ is

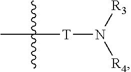

wherein R₃ and R₄ are independently selected from the group consisting of: H and C₁-C₃ alkyl, or together with the nitrogen to which they are attached form a saturated 5- or 6-membered ring having 0 or 1 additional nitrogen atoms or 1 oxygen atom, wherein the ring is optionally substituted with a C₁-C₆ alkyl group.

In yet another embodiment R₂ is

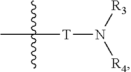

wherein R₃ and R₄ are independently selected from the group consisting of: H and C₁-C₃ alkyl, or together with the nitrogen to which they are attached form a saturated 5- or 6-membered ring having 0 or 1 additional nitrogen atoms or 1 oxygen atom, wherein the ring is optionally substituted with a C₁-C₃ alkyl group.

In a further embodiment R₂ is

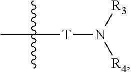

wherein R₃ and R₄ are independently selected from the group consisting of: H and methyl, or together with the nitrogen to which they are attached form a saturated 5- or 6-membered ring having 0 or 1 additional nitrogen atoms or 1 oxygen atom, wherein the ring is optionally substituted with a methyl group.

In yet another embodiment R₂ is

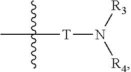

wherein R₃ and R₄ are independently selected from the group consisting of: H and methyl, or together with the nitrogen to which they are attached form a saturated 5- or 6-membered ring selected from:

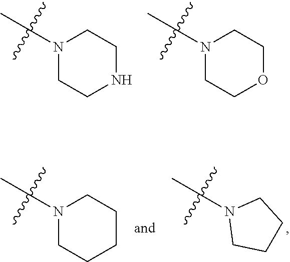 [SMILES: *N1CCNCC1 ; *N1CCOCC1 ; *N1CCCCC1 ; *N1CCCC1]

wherein each ring is optionally substituted with a methyl group.

In yet another embodiment R₂ is

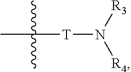

wherein R₃ and R₄ are independently selected from the group consisting of: H and methyl, or together with the nitrogen to which they are attached form a saturated 5- or 6-membered ring selected from:

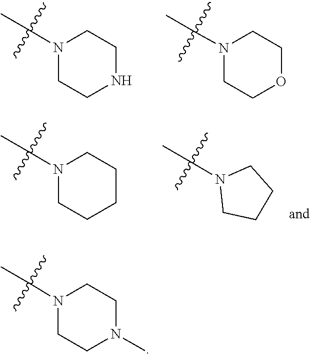

In some embodiments T is a straight-chain or branched-chain alkanediyl group having between 1 and 5 carbon atoms.

In some embodiments T is a straight-chain or branched-chain alkanediyl group having between 1 and 4 carbon atoms.

In other embodiments T is a straight-chain or branched-chain alkanediyl group having between 1 and 3 carbon atoms.

In other embodiments T is a straight-chain or branched-chain alkanediyl group having 1 or 2 carbon atoms.

In some embodiments R₂ is selected from the group consisting of:

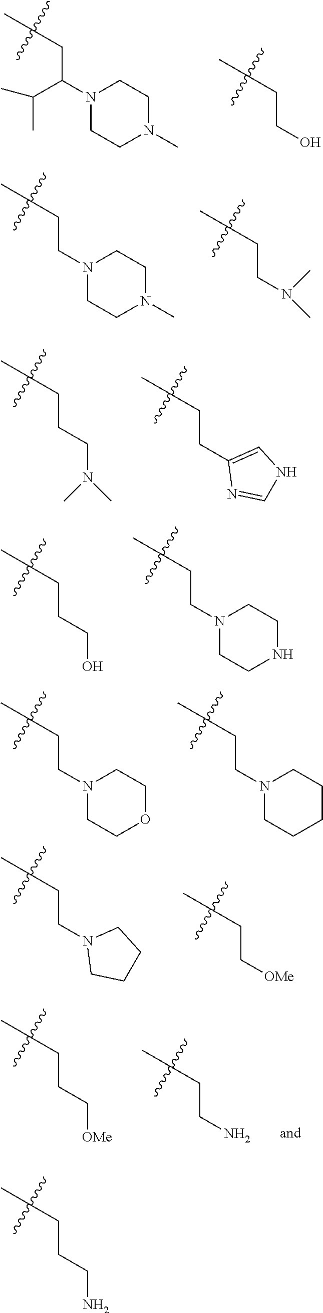

R₂ may be selected from any one or a combination of the above groups. For example, in an alternative embodiment R₂ is selected from the group consisting of:

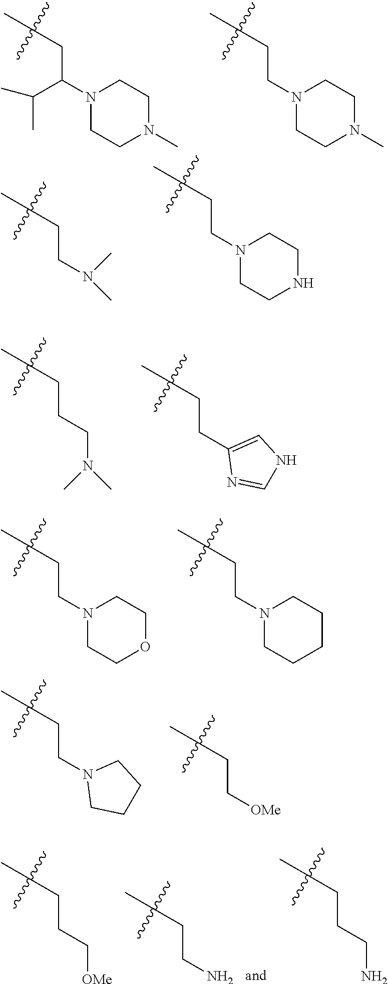

In another alternative embodiment R₂ is selected from the group consisting of:

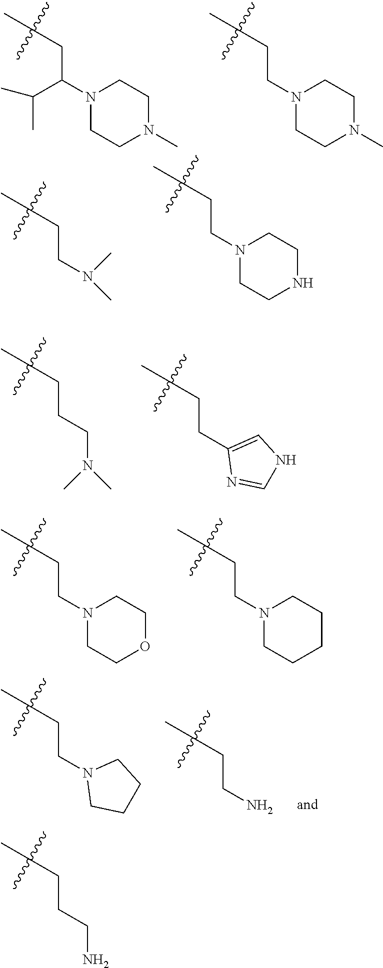

In a further alternative embodiment R₂ is selected from the group consisting of:

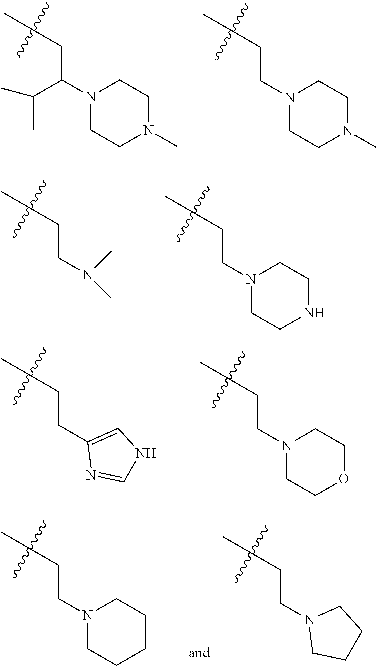

In still a further embodiment R₂ is selected from the group consisting of:

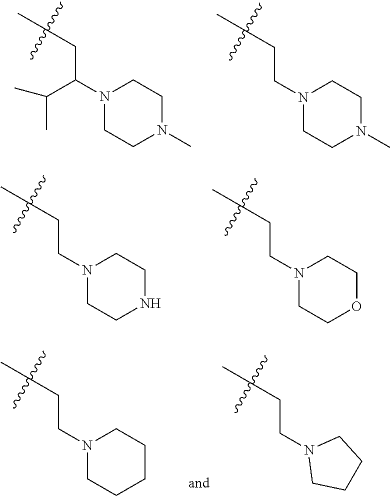

In still another embodiment R₂ is selected from the group consisting of:

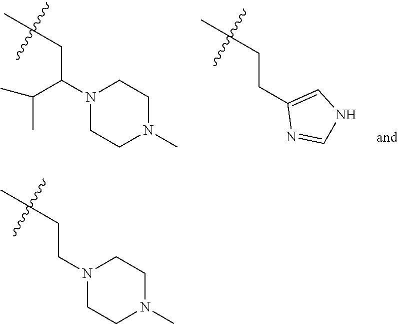

In yet another embodiment R₂ is:

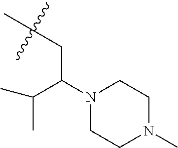 [SMILES: *CC(C(C)C)N1CCN(C)CC1]

In yet another embodiment R₂ is:

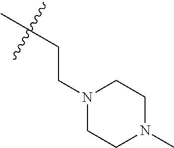 [SMILES: *CCN1CCN(C)CC1]

In still a further embodiment R₂ is:

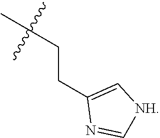 [SMILES: *CCc1c[nH]cn1]

In another embodiment R₂ is:

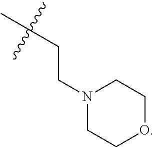 [SMILES: *CCN1CCOCC1]

In still a further embodiment R₂ is:

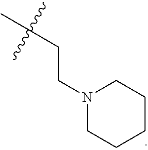 [SMILES: *CCN1CCCCC1]

In yet another embodiment R₂ is:

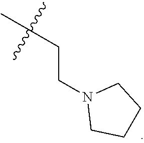 [SMILES: *CCN1CCCC1]

In still a further embodiment R₂ is:

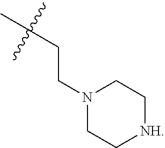 [SMILES: *CCN1CCNCC1]

In yet a further embodiment R₂ is:

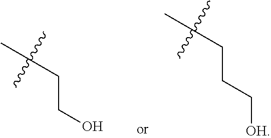

In another embodiment R₂ is:

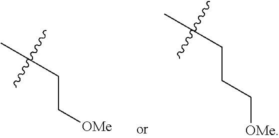 [SMILES: *CCOC ; *CCCOC]

In a further embodiment R₂ is:

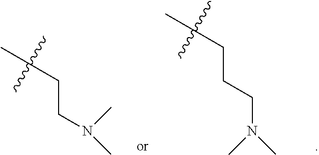

In yet another embodiment R₂ is:

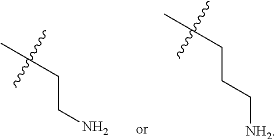

In one embodiment, the present disclosure provides a method for the treatment of cancer in a subject in need thereof, the method comprising administration to the subject of a therapeutically effective amount of a compound of the following formula (I)

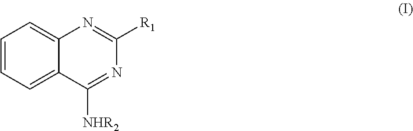

or a pharmaceutically acceptable salt, hydrate, derivative, solvate or prodrug thereof, wherein:
R₁ is selected from the group consisting of:
(i) a heteroaryl group having between 5 and 14 ring atoms in which one or more of the ring atoms are selected from nitrogen, sulfur and oxygen, a heterocyclyl group having between 5 and 14 ring atoms in which one or more of the ring atoms are selected from nitrogen and oxygen,
(ii)

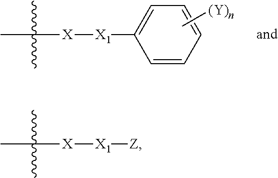

(iii)
wherein X is O, S or NH;
X₁ is absent or is a straight-chain or branched-chain alkanediyl group having between 1 and 6 carbon atoms;
Y is selected from the group consisting of: CN, NR₅R₆, OH, C₁-C₆alkoxy, halo, CF₃ and C₁-C₆ alkyl;
Z is selected from the group consisting of:a heteroaryl group having 5 or 6 ring atoms in which one or more of the ring atoms are nitrogen, oxygen or sulfur, and (methylenedioxy)phenyl, and wherein the heteroaryl group is optionally substituted with a methyl group;
n is 0, 1, 2 or 3;
R₅ and Rₑ are independently selected from the group consisting of: H and C₁-C₆ alkyl;
R₂ is selected from the group consisting of:
(i) T-heteroaryl, wherein the heteroaryl group has 5 ring atoms in which one or more of the ring atoms are nitrogen;
(ii) T-OH
(iii) T-OCH₃
(iv) T-NH₂ and,
(v)

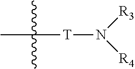

wherein T is a straight-chain or branched-chain alkanediyl group having between 1 and 10 carbon atoms;
R₃ and R₄ are independently selected from the group consisting of: H and C₁-C₆ alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group, with the proviso that the following compound is disclaimed:

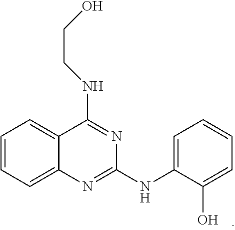 [SMILES: OCCNc1nc(Nc2ccccc2O)nc2ccccc12]

Embodiments in paragraphs [0042] to [00111] above also apply to the embodiment in paragraph [00112].

Exemplary compounds according to formula (I) include:

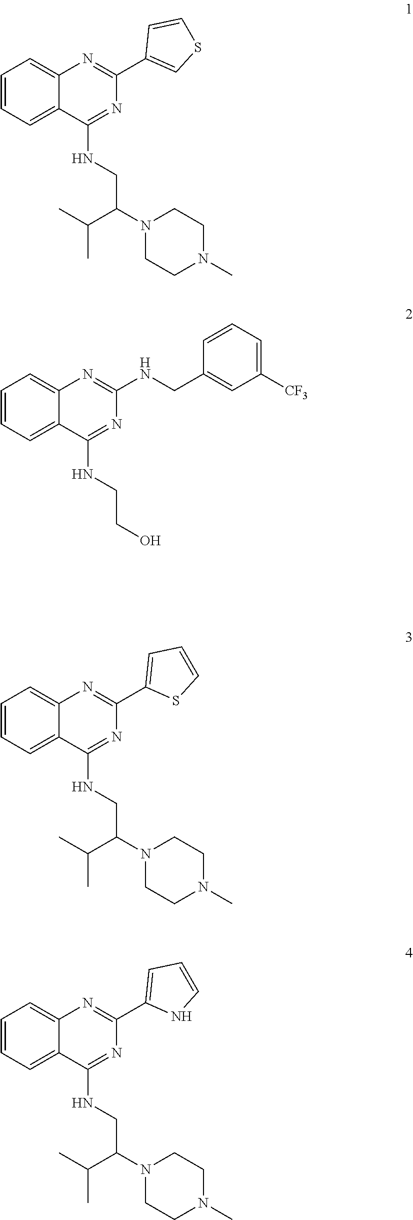 [SMILES: CC(C)C(CNc1nc(-c2ccsc2)nc2ccccc12)N1CCN(C)CC1 ; OCCNc1nc(NCc2cccc(C(F)(F)F)c2)nc2ccccc12 ; CC(C)C(CNc1nc(-c2cccs2)nc2ccccc12)N1CCN(C)CC1 ; CC(C)C(CNc1nc(-c2ccc[nH]2)nc2ccccc12)N1CCN(C)CC1]

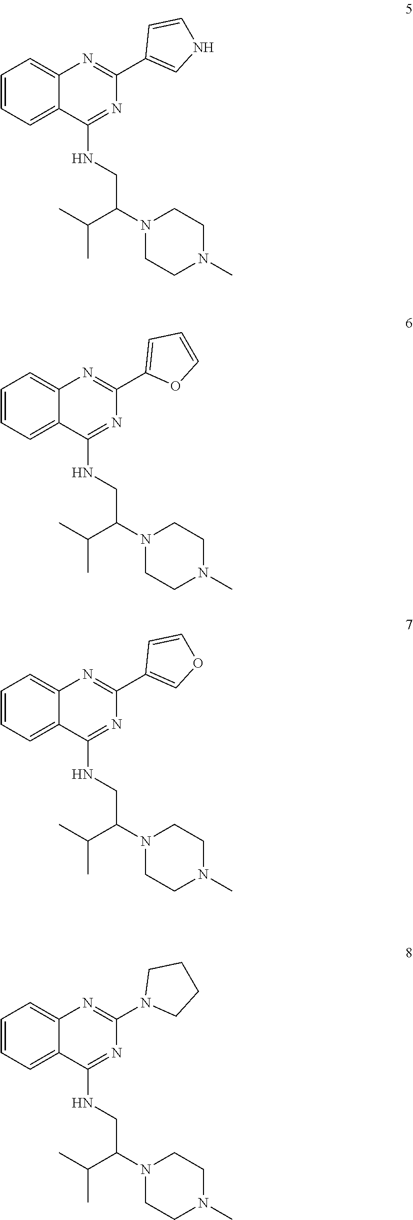

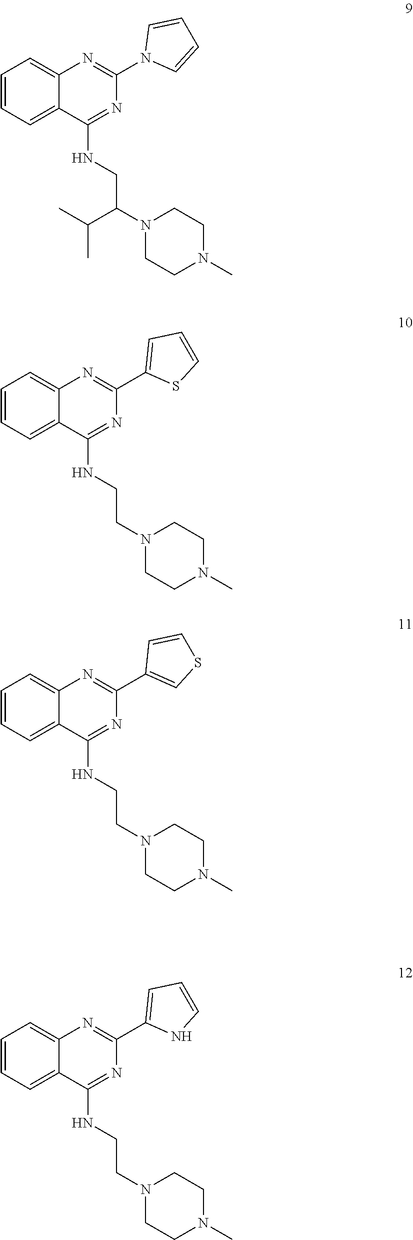

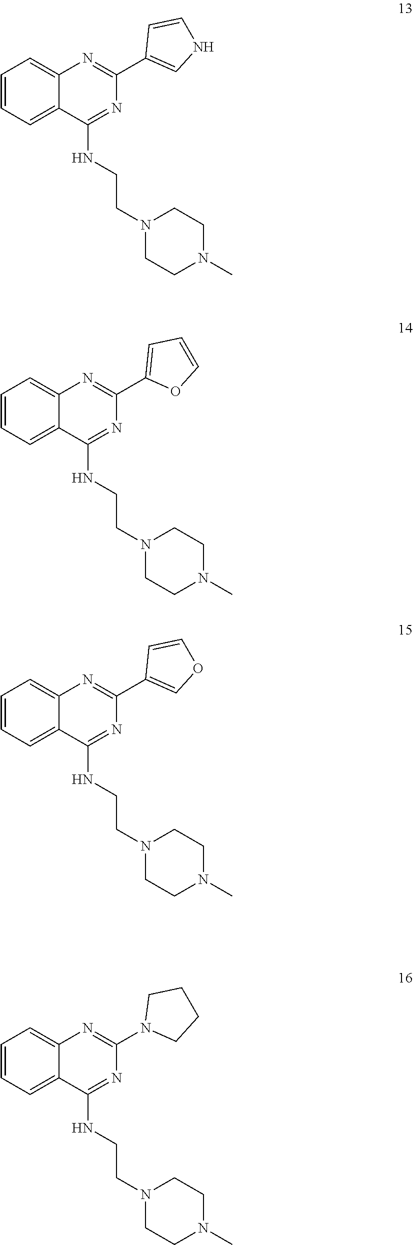

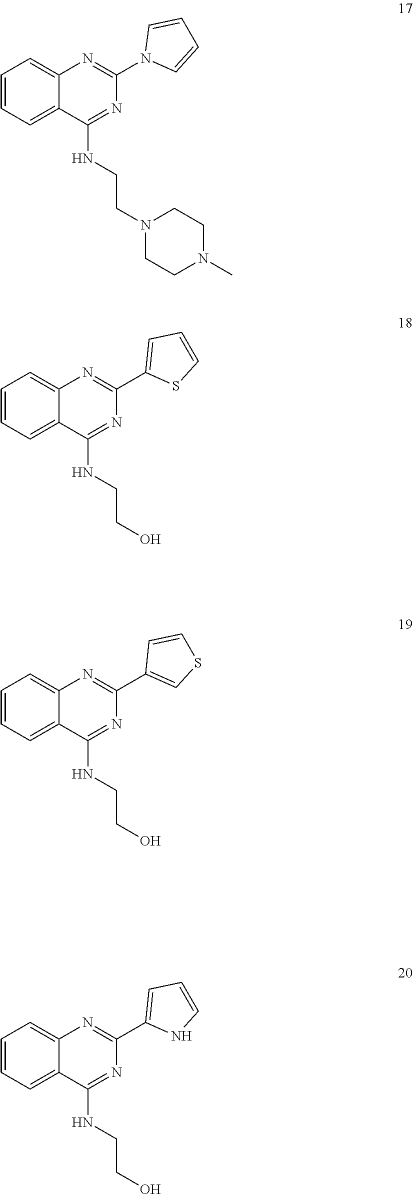 [SMILES: CN1CCN(CCNc2nc(-n3cccc3)nc3ccccc23)CC1 ; OCCNc1nc(-c2cccs2)nc2ccccc12 ; OCCNc1nc(-c2ccsc2)nc2ccccc12 ; OCCNc1nc(-c2ccc[nH]2)nc2ccccc12]

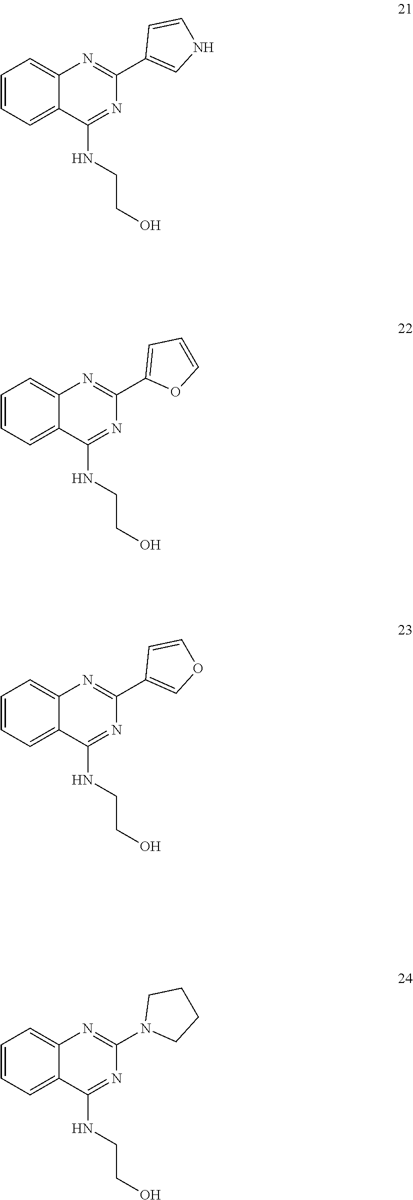 [SMILES: OCCNc1nc(-c2cc[nH]c2)nc2ccccc12 ; OCCNc1nc(-c2ccco2)nc2ccccc12 ; OCCNc1nc(-c2ccoc2)nc2ccccc12 ; OCCNc1nc(N2CCCC2)nc2ccccc12]

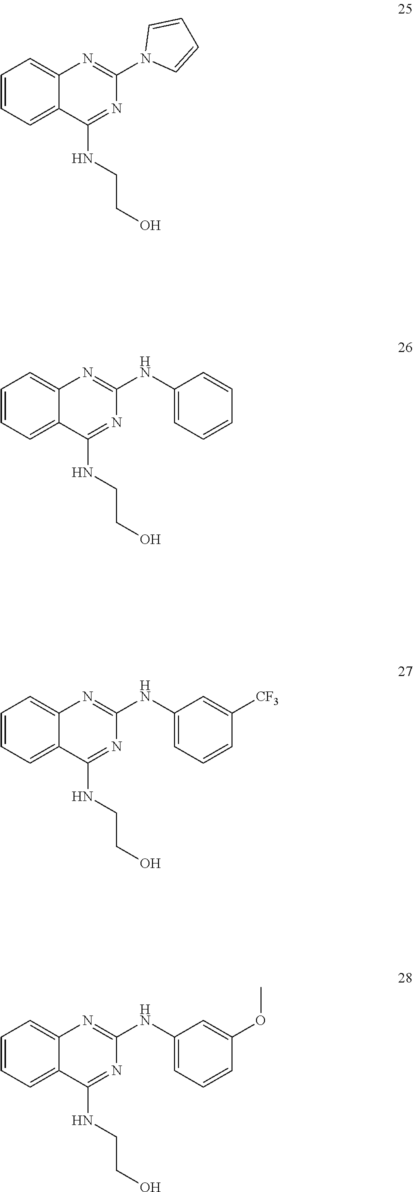 [SMILES: OCCNc1nc(-n2cccc2)nc2ccccc12 ; OCCNc1nc(Nc2ccccc2)nc2ccccc12 ; OCCNc1nc(Nc2cccc(C(F)(F)F)c2)nc2ccccc12 ; COc1cccc(Nc2nc(NCCO)c3ccccc3n2)c1]

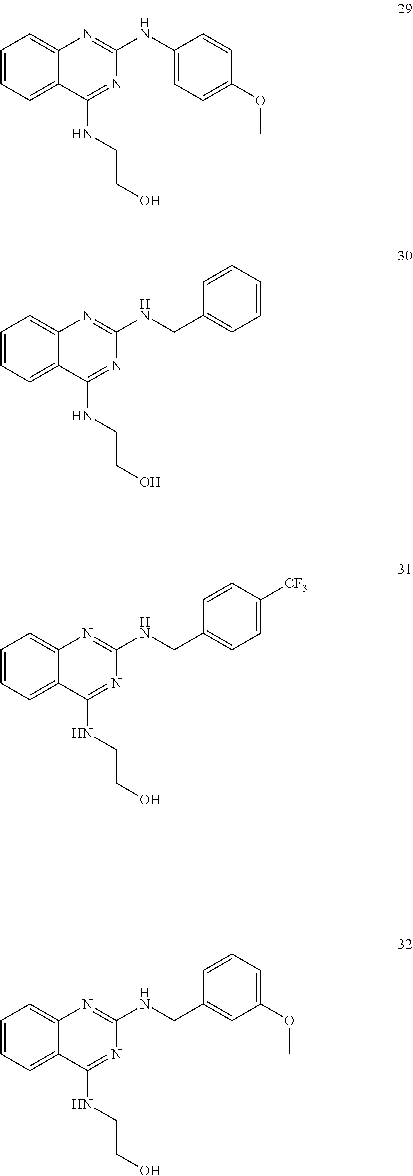 [SMILES: COc1ccc(Nc2nc(NCCO)c3ccccc3n2)cc1 ; OCCNc1nc(NCc2ccccc2)nc2ccccc12 ; OCCNc1nc(NCc2ccc(C(F)(F)F)cc2)nc2ccccc12 ; COc1cccc(CNc2nc(NCCO)c3ccccc3n2)c1]

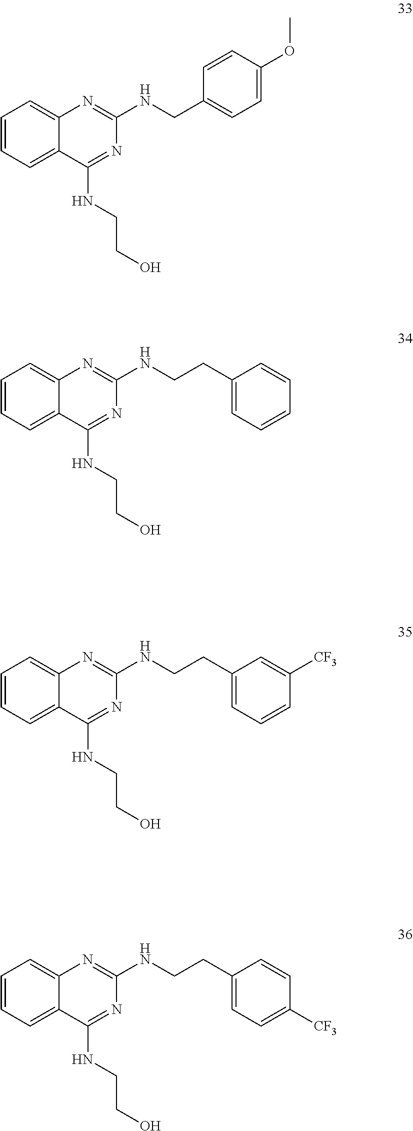 [SMILES: COc1ccc(CNc2nc(NCCO)c3ccccc3n2)cc1 ; OCCNc1nc(NCCc2ccccc2)nc2ccccc12 ; OCCNc1nc(NCCc2cccc(C(F)(F)F)c2)nc2ccccc12 ; OCCNc1nc(NCCc2ccc(C(F)(F)F)cc2)nc2ccccc12]

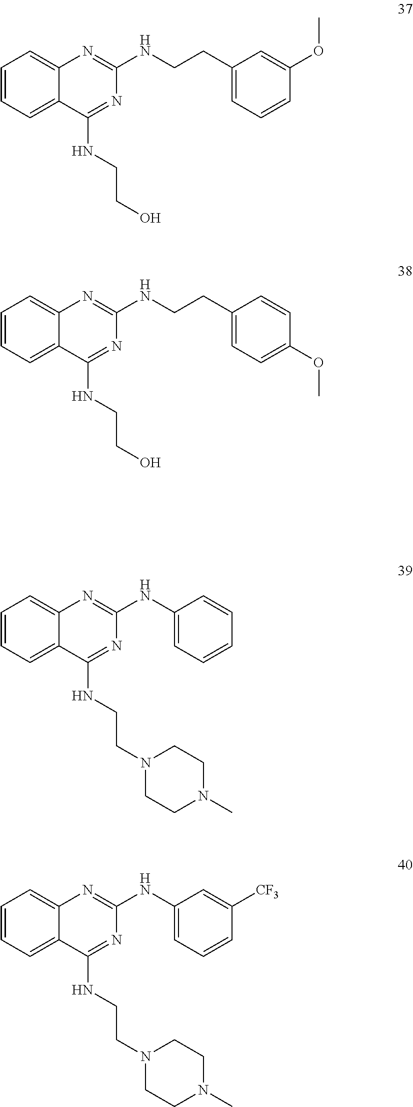 [SMILES: COc1cccc(CCNc2nc(NCCO)c3ccccc3n2)c1 ; COc1ccc(CCNc2nc(NCCO)c3ccccc3n2)cc1 ; CN1CCN(CCNc2nc(Nc3ccccc3)nc3ccccc23)CC1 ; CN1CCN(CCNc2nc(Nc3cccc(C(F)(F)F)c3)nc3ccccc23)CC1]

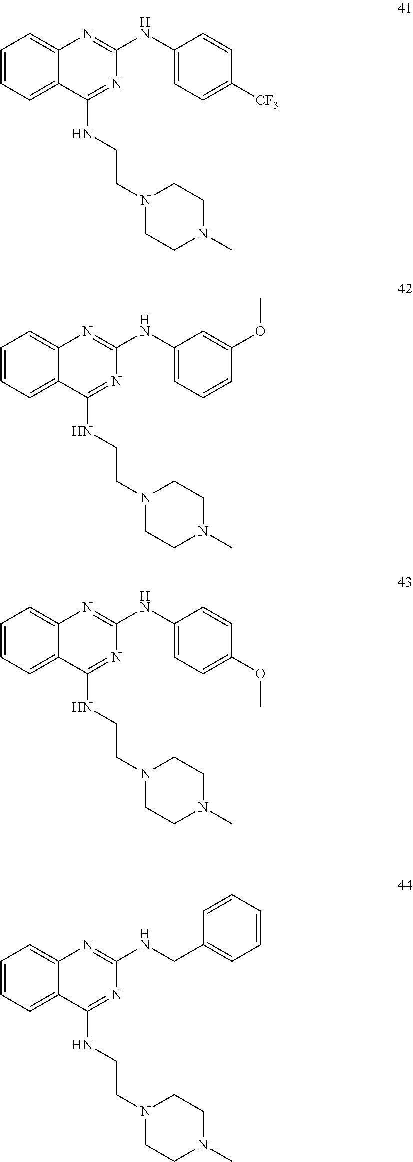 [SMILES: CN1CCN(CCNc2nc(Nc3ccc(C(F)(F)F)cc3)nc3ccccc23)CC1 ; COc1cccc(Nc2nc(NCCN3CCN(C)CC3)c3ccccc3n2)c1 ; COc1ccc(Nc2nc(NCCN3CCN(C)CC3)c3ccccc3n2)cc1 ; CN1CCN(CCNc2nc(NCc3ccccc3)nc3ccccc23)CC1]

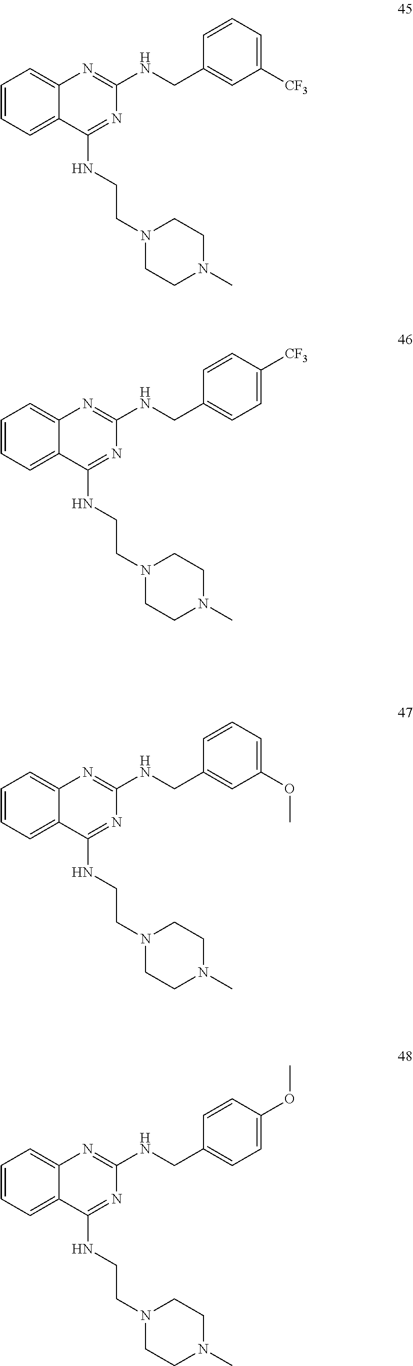 [SMILES: CN1CCN(CCNc2nc(NCc3cccc(C(F)(F)F)c3)nc3ccccc23)CC1 ; CN1CCN(CCNc2nc(NCc3ccc(C(F)(F)F)cc3)nc3ccccc23)CC1 ; COc1cccc(CNc2nc(NCCN3CCN(C)CC3)c3ccccc3n2)c1 ; COc1ccc(CNc2nc(NCCN3CCN(C)CC3)c3ccccc3n2)cc1]

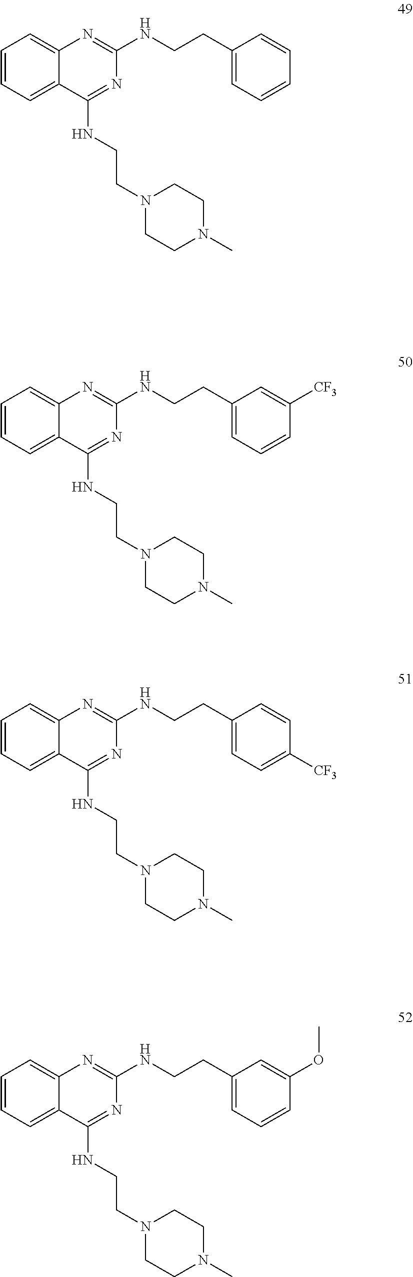 [SMILES: CN1CCN(CCNc2nc(NCCc3ccccc3)nc3ccccc23)CC1 ; CN1CCN(CCNc2nc(NCCc3cccc(C(F)(F)F)c3)nc3ccccc23)CC1 ; CN1CCN(CCNc2nc(NCCc3ccc(C(F)(F)F)cc3)nc3ccccc23)CC1 ; COc1cccc(CCNc2nc(NCCN3CCN(C)CC3)c3ccccc3n2)c1]

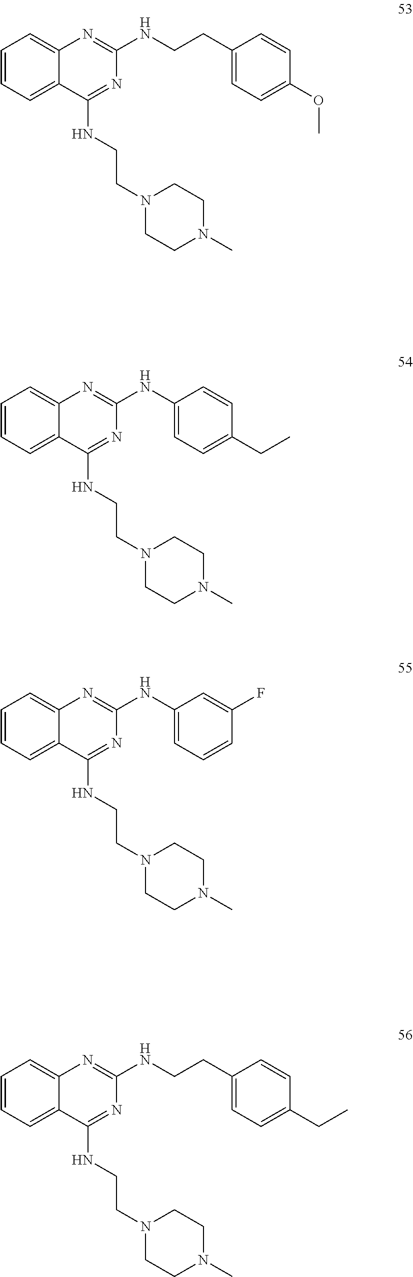 [SMILES: COc1ccc(CCNc2nc(NCCN3CCN(C)CC3)c3ccccc3n2)cc1 ; CCc1ccc(Nc2nc(NCCN3CCN(C)CC3)c3ccccc3n2)cc1 ; CN1CCN(CCNc2nc(Nc3cccc(F)c3)nc3ccccc23)CC1 ; CCc1ccc(CCNc2nc(NCCN3CCN(C)CC3)c3ccccc3n2)cc1]

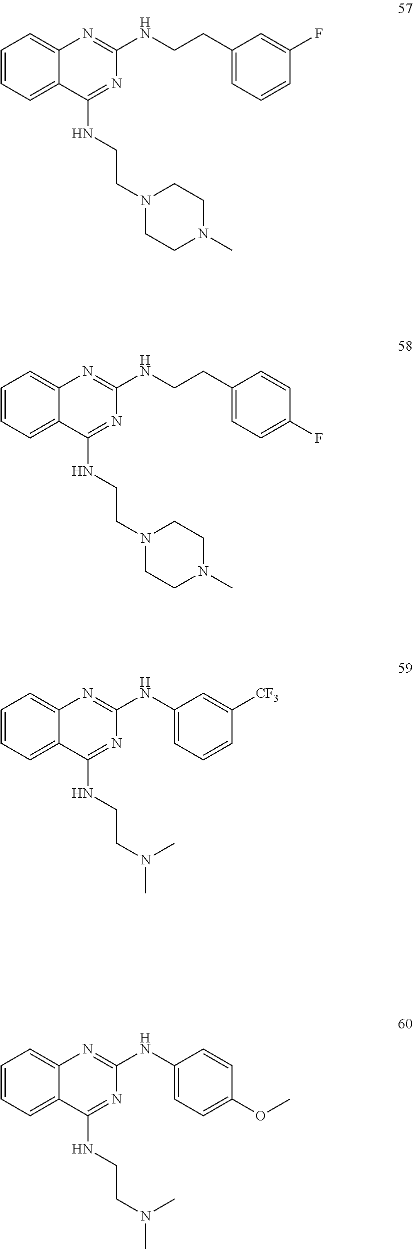 [SMILES: CN1CCN(CCNc2nc(NCCc3cccc(F)c3)nc3ccccc23)CC1 ; CN1CCN(CCNc2nc(NCCc3ccc(F)cc3)nc3ccccc23)CC1 ; CN(C)CCNc1nc(Nc2cccc(C(F)(F)F)c2)nc2ccccc12 ; COc1ccc(Nc2nc(NCCN(C)C)c3ccccc3n2)cc1]

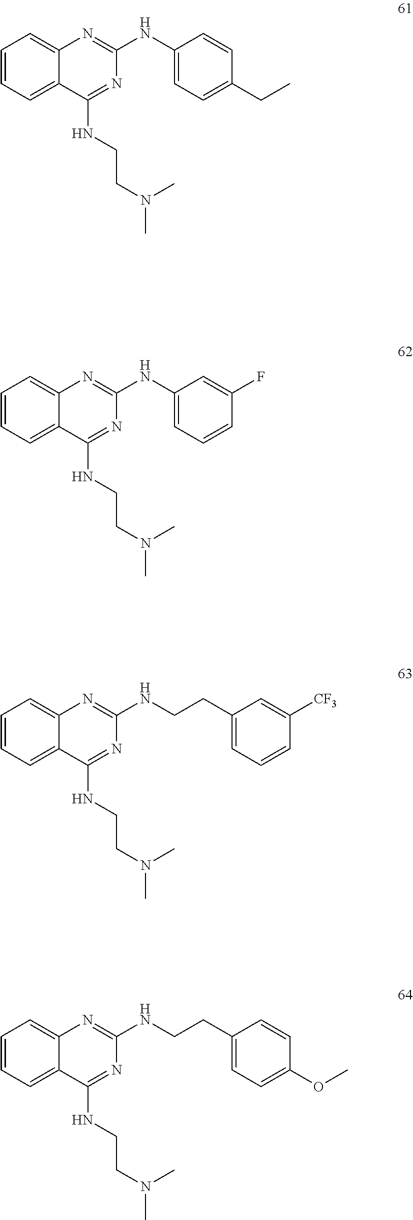 [SMILES: CCc1ccc(Nc2nc(NCCN(C)C)c3ccccc3n2)cc1 ; CN(C)CCNc1nc(Nc2cccc(F)c2)nc2ccccc12 ; CN(C)CCNc1nc(NCCc2cccc(C(F)(F)F)c2)nc2ccccc12 ; COc1ccc(CCNc2nc(NCCN(C)C)c3ccccc3n2)cc1]

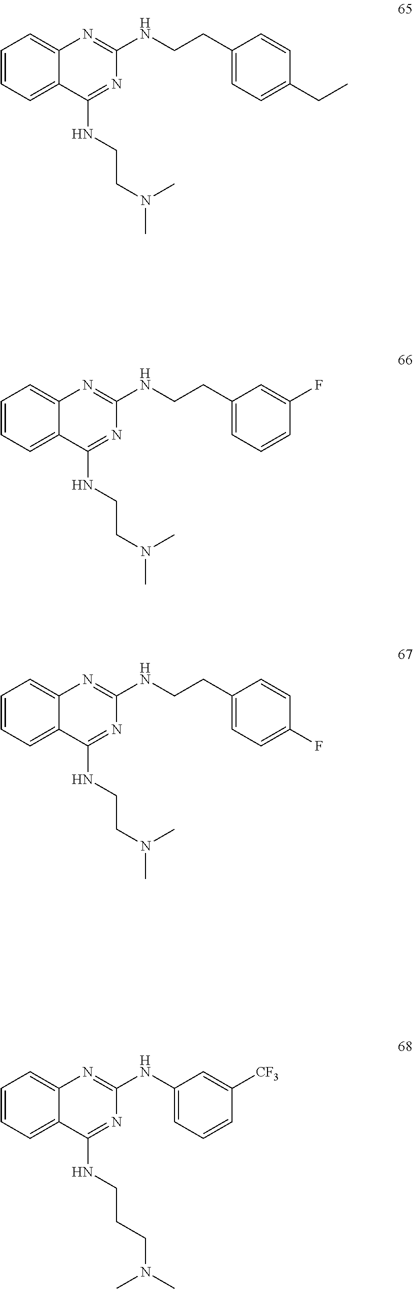 [SMILES: CCc1ccc(CCNc2nc(NCCN(C)C)c3ccccc3n2)cc1 ; CN(C)CCNc1nc(NCCc2cccc(F)c2)nc2ccccc12 ; CN(C)CCNc1nc(NCCc2ccc(F)cc2)nc2ccccc12 ; CN(C)CCCNc1nc(Nc2cccc(C(F)(F)F)c2)nc2ccccc12]

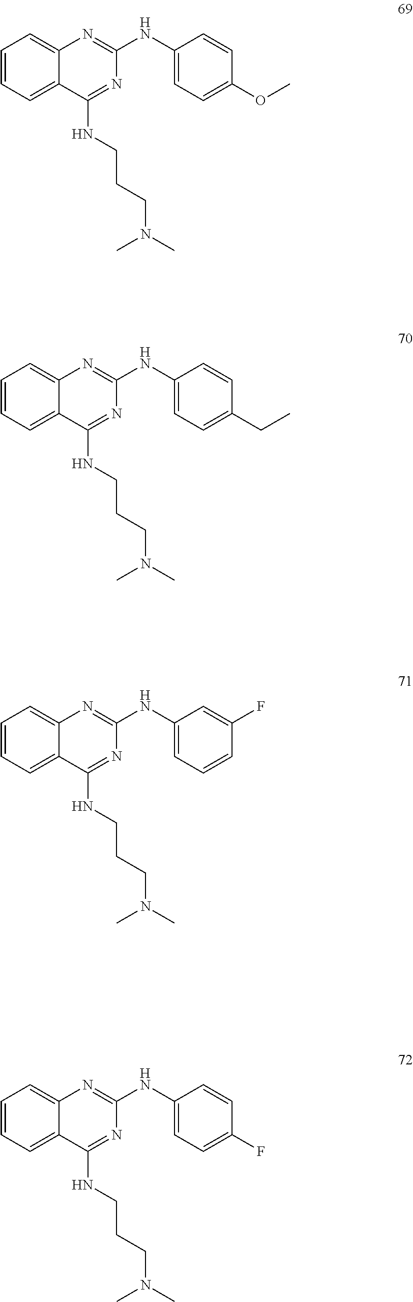 [SMILES: COc1ccc(Nc2nc(NCCCN(C)C)c3ccccc3n2)cc1 ; CCc1ccc(Nc2nc(NCCCN(C)C)c3ccccc3n2)cc1 ; CN(C)CCCNc1nc(Nc2cccc(F)c2)nc2ccccc12 ; CN(C)CCCNc1nc(Nc2ccc(F)cc2)nc2ccccc12]

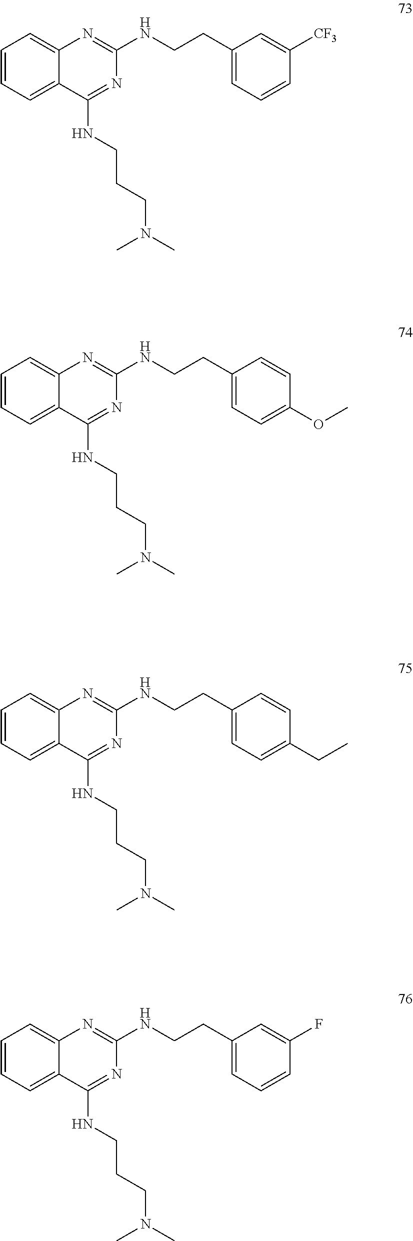 [SMILES: CN(C)CCCNc1nc(NCCc2cccc(C(F)(F)F)c2)nc2ccccc12 ; COc1ccc(CCNc2nc(NCCCN(C)C)c3ccccc3n2)cc1 ; CCc1ccc(CCNc2nc(NCCCN(C)C)c3ccccc3n2)cc1 ; CN(C)CCCNc1nc(NCCc2cccc(F)c2)nc2ccccc12]

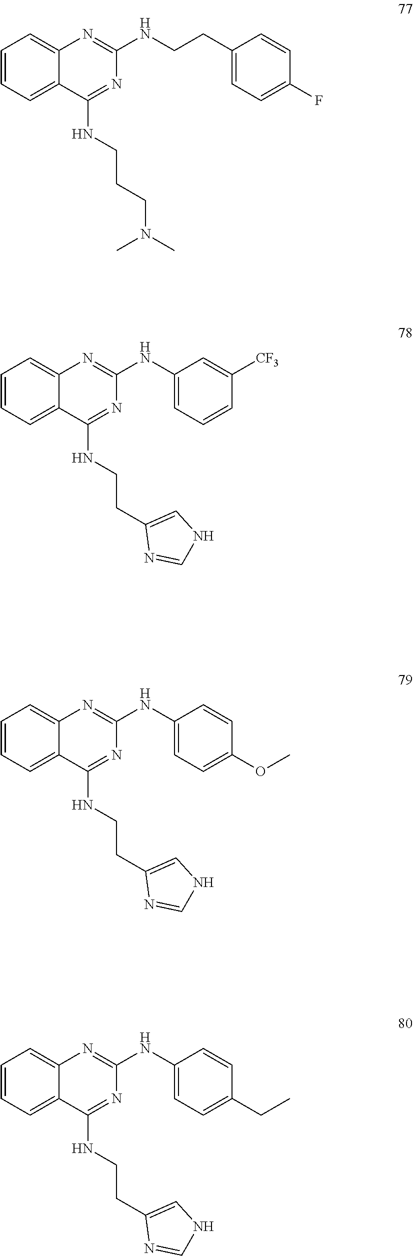 [SMILES: CN(C)CCCNc1nc(NCCc2ccc(F)cc2)nc2ccccc12 ; FC(F)(F)c1cccc(Nc2nc(NCCc3c[nH]cn3)c3ccccc3n2)c1 ; COc1ccc(Nc2nc(NCCc3c[nH]cn3)c3ccccc3n2)cc1 ; CCc1ccc(Nc2nc(NCCc3c[nH]cn3)c3ccccc3n2)cc1]

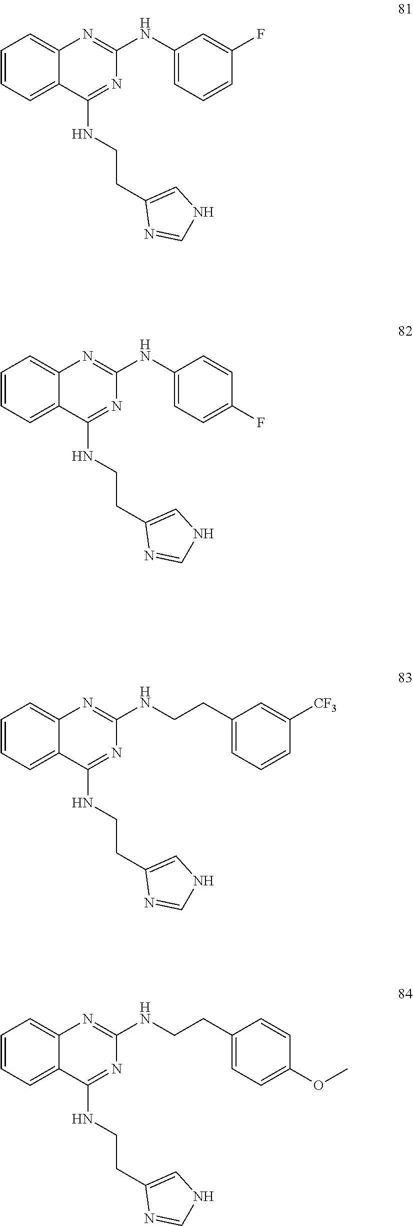 [SMILES: Fc1cccc(Nc2nc(NCCc3c[nH]cn3)c3ccccc3n2)c1 ; Fc1ccc(Nc2nc(NCCc3c[nH]cn3)c3ccccc3n2)cc1 ; FC(F)(F)c1cccc(CCNc2nc(NCCc3c[nH]cn3)c3ccccc3n2)c1 ; COc1ccc(CCNc2nc(NCCc3c[nH]cn3)c3ccccc3n2)cc1]

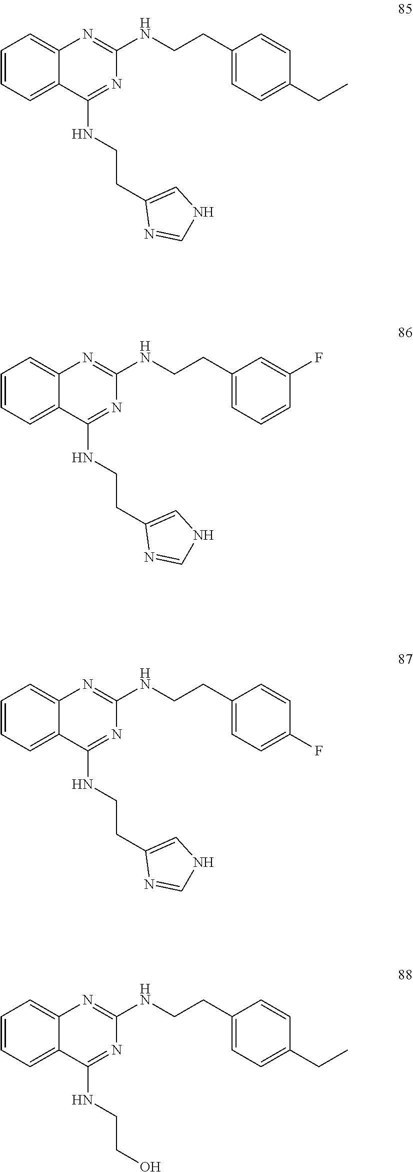 [SMILES: CCc1ccc(CCNc2nc(NCCc3c[nH]cn3)c3ccccc3n2)cc1 ; Fc1cccc(CCNc2nc(NCCc3c[nH]cn3)c3ccccc3n2)c1 ; Fc1ccc(CCNc2nc(NCCc3c[nH]cn3)c3ccccc3n2)cc1 ; CCc1ccc(CCNc2nc(NCCO)c3ccccc3n2)cc1]

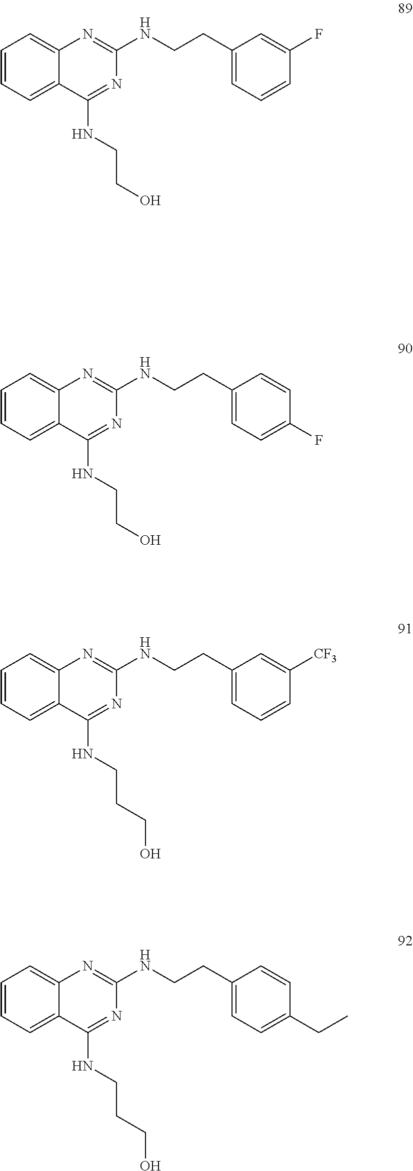 [SMILES: OCCNc1nc(NCCc2cccc(F)c2)nc2ccccc12 ; OCCNc1nc(NCCc2ccc(F)cc2)nc2ccccc12 ; OCCCNc1nc(NCCc2cccc(C(F)(F)F)c2)nc2ccccc12 ; CCc1ccc(CCNc2nc(NCCCO)c3ccccc3n2)cc1]

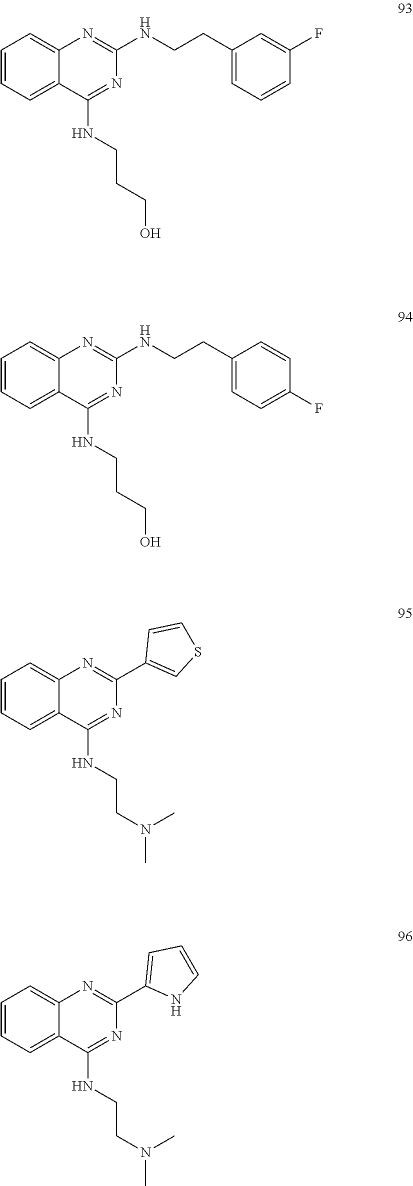 [SMILES: OCCCNc1nc(NCCc2cccc(F)c2)nc2ccccc12 ; OCCCNc1nc(NCCc2ccc(F)cc2)nc2ccccc12 ; CN(C)CCNc1nc(-c2ccsc2)nc2ccccc12 ; CN(C)CCNc1nc(-c2ccc[nH]2)nc2ccccc12]

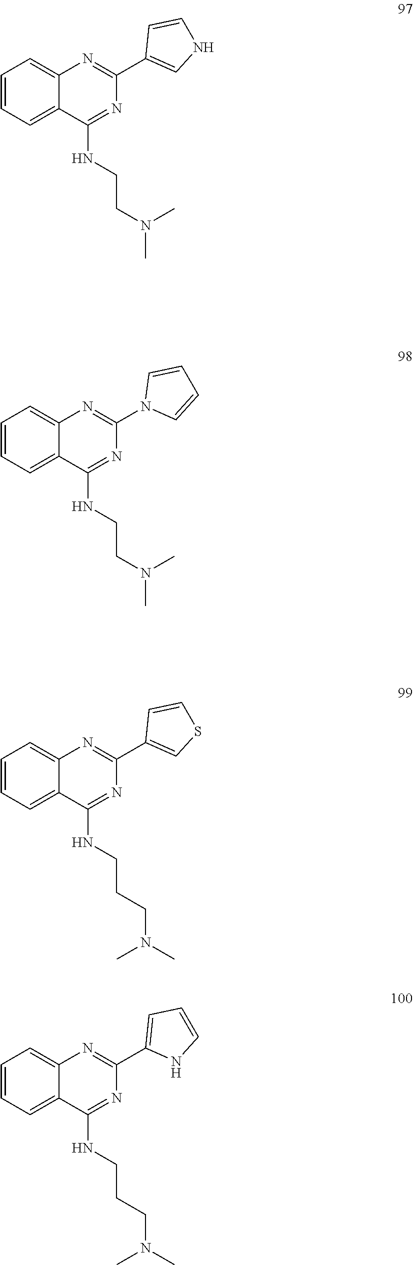 [SMILES: CN(C)CCNc1nc(-c2cc[nH]c2)nc2ccccc12 ; CN(C)CCNc1nc(-n2cccc2)nc2ccccc12 ; CN(C)CCCNc1nc(-c2ccsc2)nc2ccccc12 ; CN(C)CCCNc1nc(-c2ccc[nH]2)nc2ccccc12]

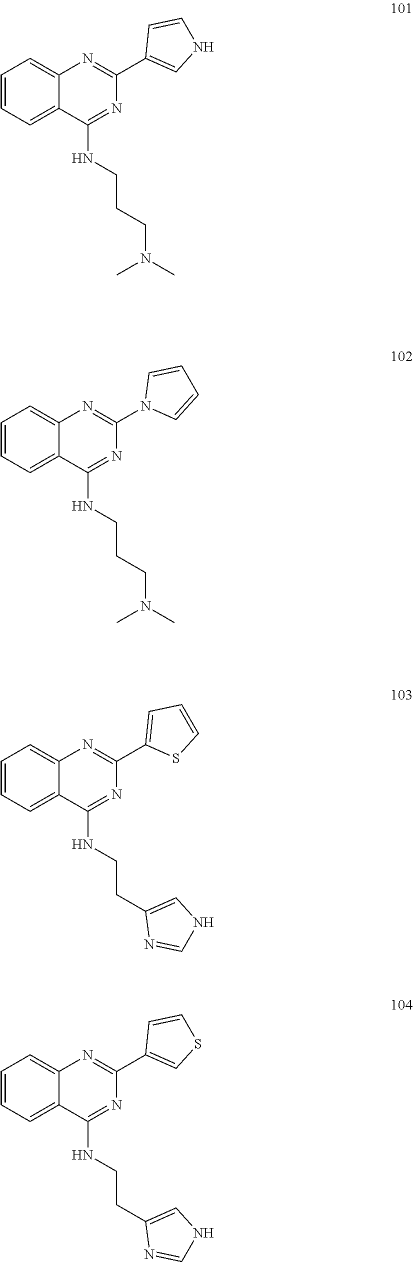 [SMILES: CN(C)CCCNc1nc(-c2cc[nH]c2)nc2ccccc12 ; CN(C)CCCNc1nc(-n2cccc2)nc2ccccc12 ; c1csc(-c2nc(NCCc3c[nH]cn3)c3ccccc3n2)c1 ; c1ccc2c(NCCc3c[nH]cn3)nc(-c3ccsc3)nc2c1]

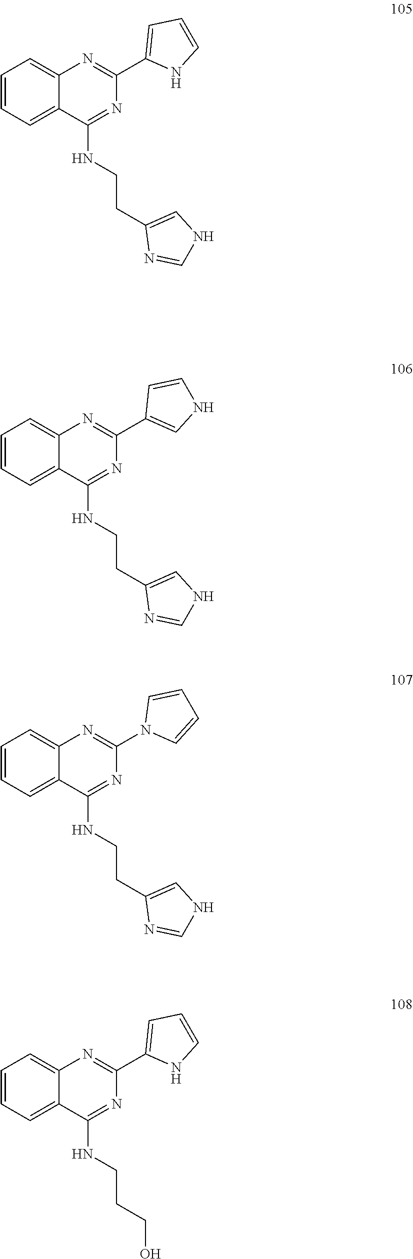 [SMILES: c1c[nH]c(-c2nc(NCCc3c[nH]cn3)c3ccccc3n2)c1 ; c1ccc2c(NCCc3c[nH]cn3)nc(-c3cc[nH]c3)nc2c1 ; c1ccc2c(NCCc3c[nH]cn3)nc(-n3cccc3)nc2c1 ; OCCCNc1nc(-c2ccc[nH]2)nc2ccccc12]

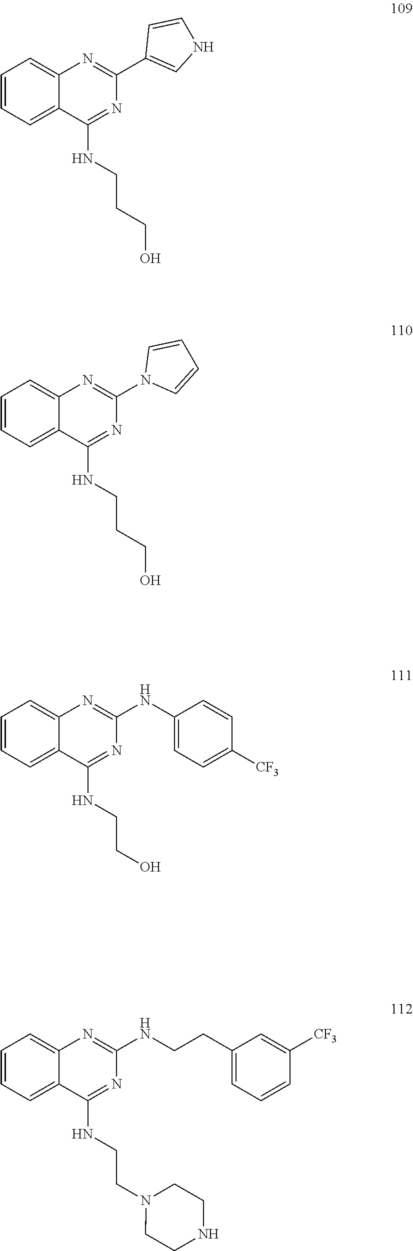 [SMILES: OCCCNc1nc(-c2cc[nH]c2)nc2ccccc12 ; OCCCNc1nc(-n2cccc2)nc2ccccc12 ; OCCNc1nc(Nc2ccc(C(F)(F)F)cc2)nc2ccccc12 ; FC(F)(F)c1cccc(CCNc2nc(NCCN3CCNCC3)c3ccccc3n2)c1]

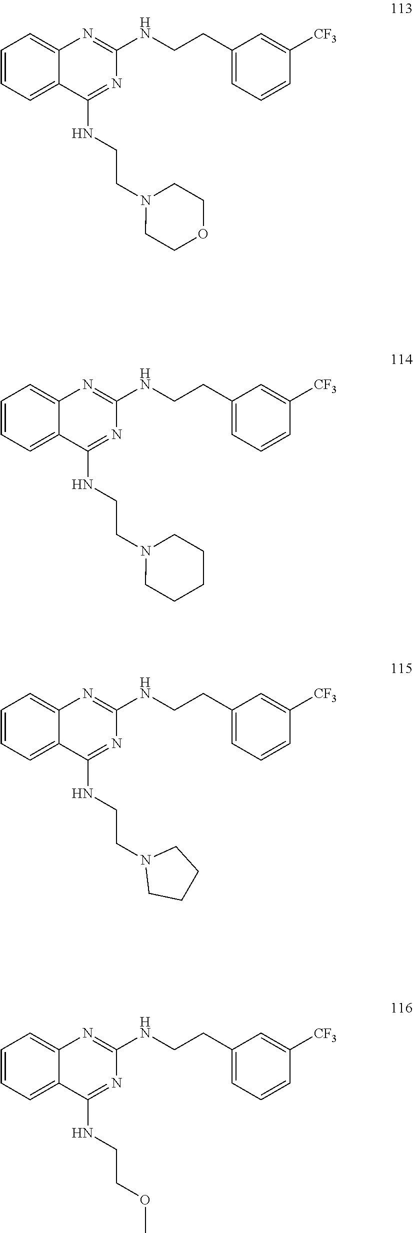 [SMILES: FC(F)(F)c1cccc(CCNc2nc(NCCN3CCOCC3)c3ccccc3n2)c1 ; FC(F)(F)c1cccc(CCNc2nc(NCCN3CCCCC3)c3ccccc3n2)c1 ; FC(F)(F)c1cccc(CCNc2nc(NCCN3CCCC3)c3ccccc3n2)c1 ; COCCNc1nc(NCCc2cccc(C(F)(F)F)c2)nc2ccccc12]

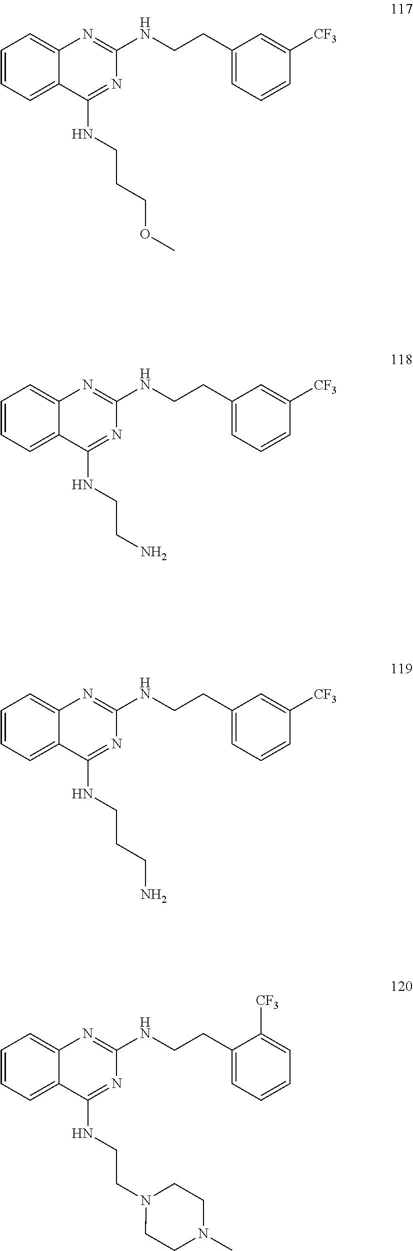 [SMILES: COCCCNc1nc(NCCc2cccc(C(F)(F)F)c2)nc2ccccc12 ; NCCNc1nc(NCCc2cccc(C(F)(F)F)c2)nc2ccccc12 ; NCCCNc1nc(NCCc2cccc(C(F)(F)F)c2)nc2ccccc12 ; CN1CCN(CCNc2nc(NCCc3ccccc3C(F)(F)F)nc3ccccc23)CC1]

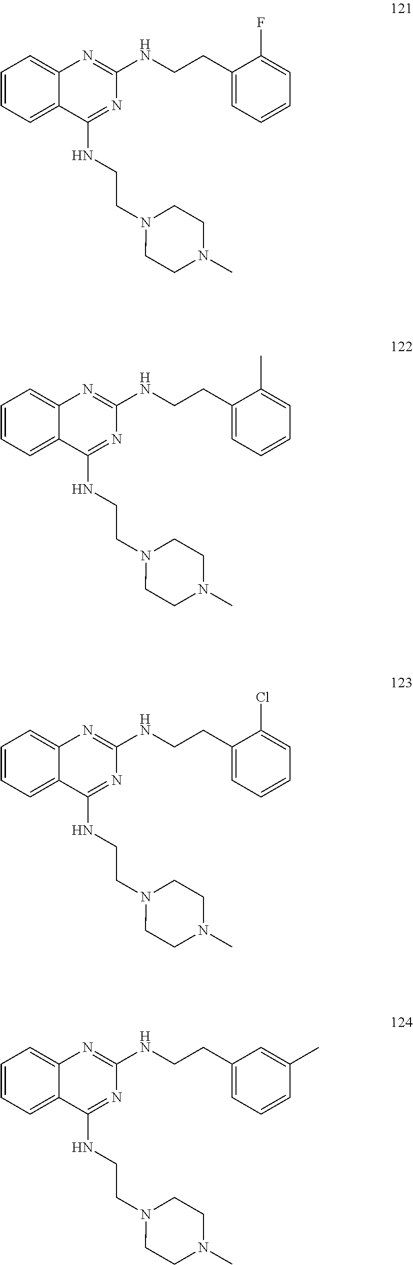 [SMILES: CN1CCN(CCNc2nc(NCCc3ccccc3F)nc3ccccc23)CC1 ; Cc1ccccc1CCNc1nc(NCCN2CCN(C)CC2)c2ccccc2n1 ; CN1CCN(CCNc2nc(NCCc3ccccc3Cl)nc3ccccc23)CC1 ; Cc1cccc(CCNc2nc(NCCN3CCN(C)CC3)c3ccccc3n2)c1]

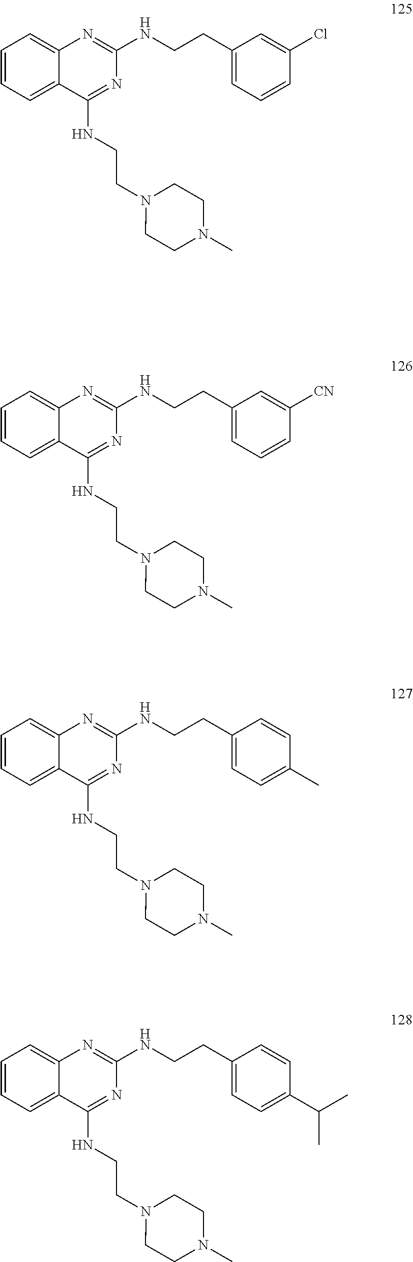 [SMILES: CN1CCN(CCNc2nc(NCCc3cccc(Cl)c3)nc3ccccc23)CC1 ; CN1CCN(CCNc2nc(NCCc3cccc(C#N)c3)nc3ccccc23)CC1 ; Cc1ccc(CCNc2nc(NCCN3CCN(C)CC3)c3ccccc3n2)cc1 ; CC(C)c1ccc(CCNc2nc(NCCN3CCN(C)CC3)c3ccccc3n2)cc1]

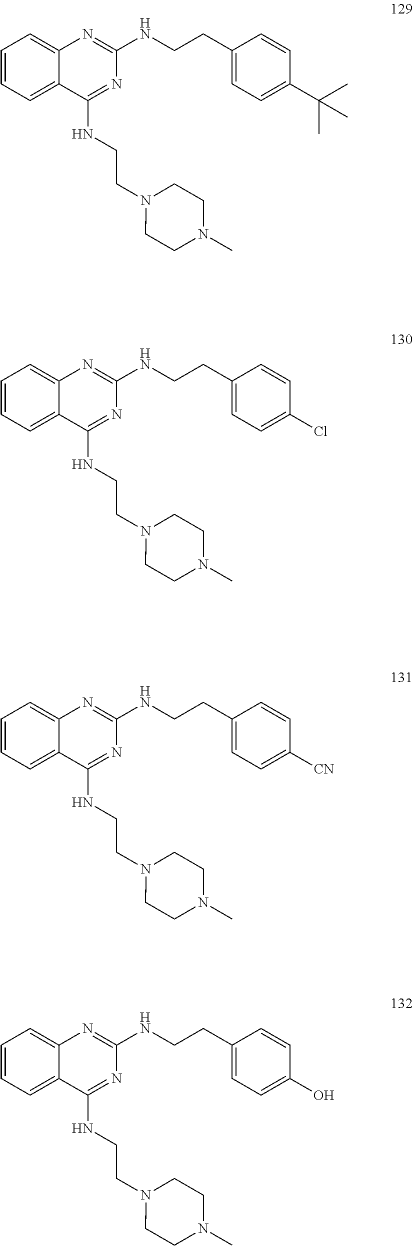 [SMILES: CN1CCN(CCNc2nc(NCCc3ccc(C(C)(C)C)cc3)nc3ccccc23)CC1 ; CN1CCN(CCNc2nc(NCCc3ccc(Cl)cc3)nc3ccccc23)CC1 ; CN1CCN(CCNc2nc(NCCc3ccc(C#N)cc3)nc3ccccc23)CC1 ; CN1CCN(CCNc2nc(NCCc3ccc(O)cc3)nc3ccccc23)CC1]

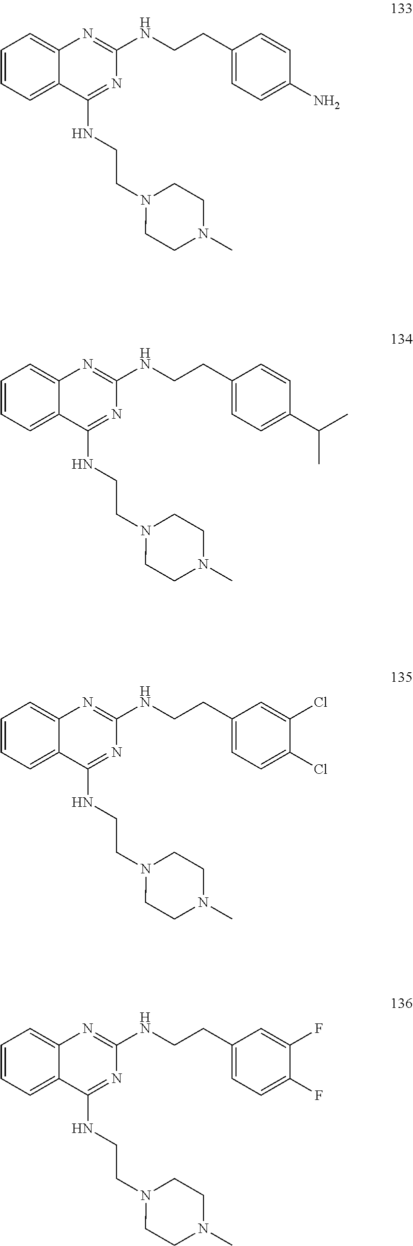 [SMILES: CN1CCN(CCNc2nc(NCCc3ccc(N)cc3)nc3ccccc23)CC1 ; CC(C)c1ccc(CCNc2nc(NCCN3CCN(C)CC3)c3ccccc3n2)cc1 ; CN1CCN(CCNc2nc(NCCc3ccc(Cl)c(Cl)c3)nc3ccccc23)CC1 ; CN1CCN(CCNc2nc(NCCc3ccc(F)c(F)c3)nc3ccccc23)CC1]

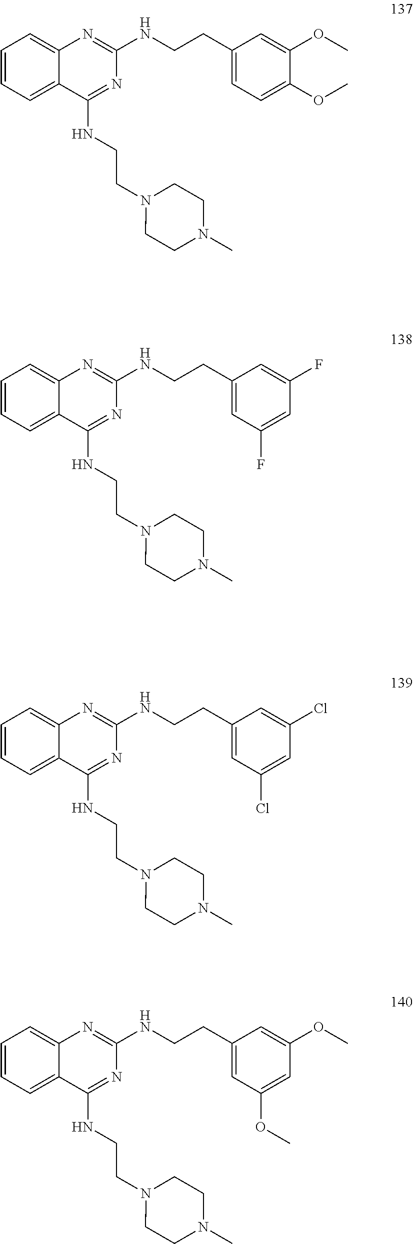 [SMILES: COc1ccc(CCNc2nc(NCCN3CCN(C)CC3)c3ccccc3n2)cc1OC ; CN1CCN(CCNc2nc(NCCc3cc(F)cc(F)c3)nc3ccccc23)CC1 ; CN1CCN(CCNc2nc(NCCc3cc(Cl)cc(Cl)c3)nc3ccccc23)CC1 ; COc1cc(CCNc2nc(NCCN3CCN(C)CC3)c3ccccc3n2)cc(OC)c1]

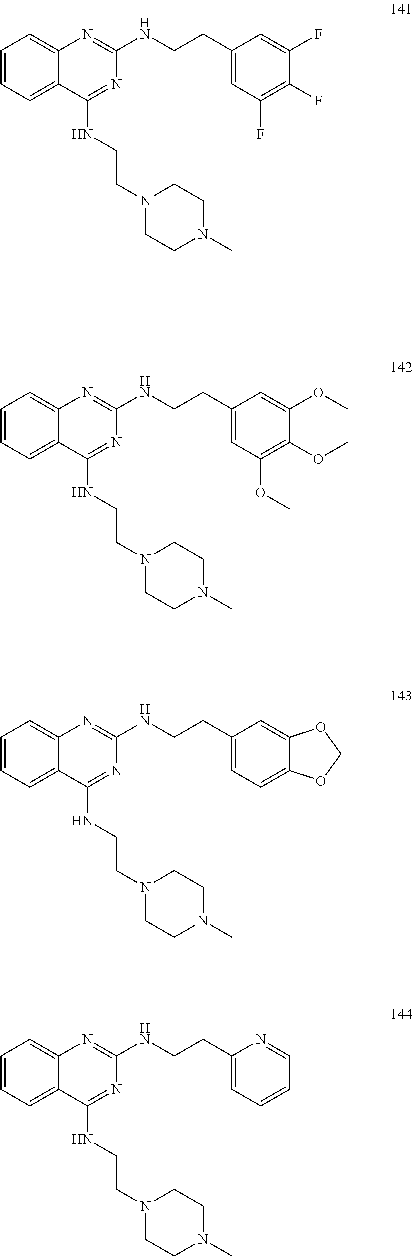 [SMILES: CN1CCN(CCNc2nc(NCCc3cc(F)c(F)c(F)c3)nc3ccccc23)CC1 ; COc1cc(CCNc2nc(NCCN3CCN(C)CC3)c3ccccc3n2)cc(OC)c1OC ; CN1CCN(CCNc2nc(NCCc3ccc4c(c3)OCO4)nc3ccccc23)CC1 ; CN1CCN(CCNc2nc(NCCc3ccccn3)nc3ccccc23)CC1]

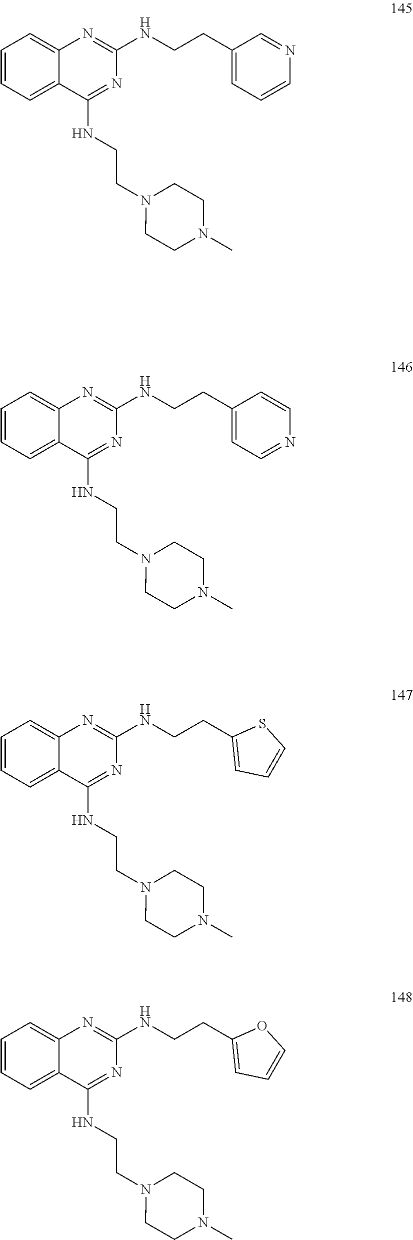 [SMILES: CN1CCN(CCNc2nc(NCCc3cccnc3)nc3ccccc23)CC1 ; CN1CCN(CCNc2nc(NCCc3ccncc3)nc3ccccc23)CC1 ; CN1CCN(CCNc2nc(NCCc3cccs3)nc3ccccc23)CC1 ; CN1CCN(CCNc2nc(NCCc3ccco3)nc3ccccc23)CC1]

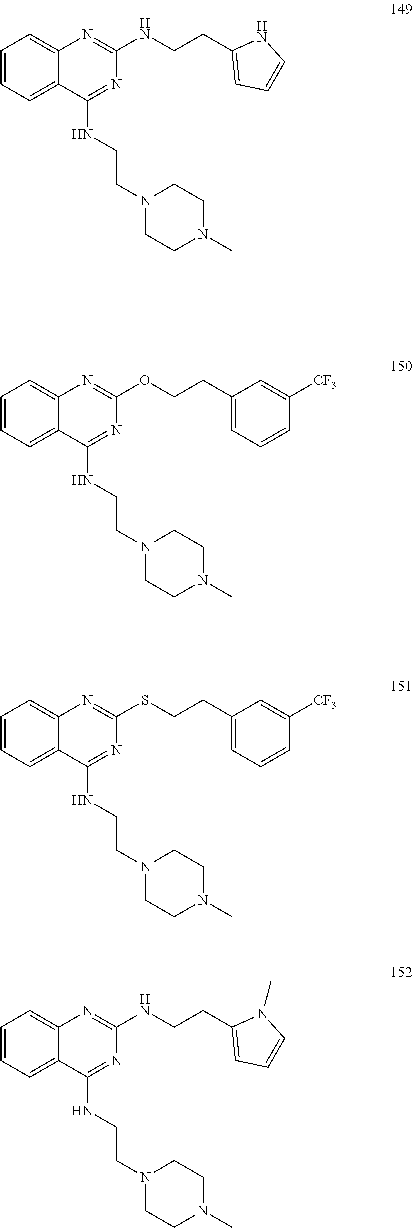 [SMILES: CN1CCN(CCNc2nc(NCCc3ccc[nH]3)nc3ccccc23)CC1 ; CN1CCN(CCNc2nc(OCCc3cccc(C(F)(F)F)c3)nc3ccccc23)CC1 ; CN1CCN(CCNc2nc(SCCc3cccc(C(F)(F)F)c3)nc3ccccc23)CC1 ; CN1CCN(CCNc2nc(NCCc3cccn3C)nc3ccccc23)CC1]

In one embodiment the compound of formula (I) is selected from any one or more of the above compounds 1 to 151, in any combination.

In one embodiment the compound of formula (I) is selected from any one or more of the above compounds 1 to 148 and 150 to 152, in any combination.

In one embodiment the compound of formula (I) is selected from any one or more of the above compounds 1 to 55, in any combination.

Selected compounds of formula (I) may include one or more chiral centres. The present disclosure extends to all enantiomers and diastereoisomers as well as mixtures thereof in any proportions. The disclosure also extends to isolated enantiomers or pairs of enantiomers. Methods of separating enantiomers and diastereoisomers are well known to persons skilled in the art. In some embodiments compounds of the formula (I) are racemic mixtures. In other embodiments compounds of the formula (I) are present in optically pure form.

Compounds of the formula (I) are also taken to include hydrates and solvates. Solvates are complexes formed by association of molecules of a solvent with a compound of the formula (I). In the case of compounds of the formula (I) that are solids, it will be understood by those skilled in the art that such compounds may exist in different crystalline or polymorphic forms, all of which are intended to be within the scope of the present disclosure.

The compounds of formula (I) may be in the form of pharmaceutically acceptable salts. Such salts are well known to those skilled in the art. S. M. Berge et al. describe pharmaceutically acceptable salts in detail in J. Pharmaceutical Sciences, 1977, 66: 1-19. Pharmaceutically acceptable salts can be prepared in situ during the final isolation and purification of compounds of the formula (I), or separately by reacting the free base compound with a suitable organic acid. Suitable pharmaceutically acceptable acid addition salts of the compounds of the present disclosure may be prepared from an inorganic acid or from an organic acid. Examples of such inorganic acids are hydrochloric, hydrobromic. hydroiodic. nitric, carbonic, sulfuric, and phosphoric acid. Appropriate organic acids may be selected from aliphatic, cycioaliphatic, aromatic, heterocyclic carboxylic and sulfonic classes of organic acids, examples of which are formic, acetic, propionic, succinic, giycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, pamoic, pantothenic, sulfanilic, cyclohexylaminosulfonic, stearic, algenic, p-hydroxybutyric and galacturonic acids. Suitable pharmaceutically acceptable base addition salts of the compounds of the present disclosure include metallic salts made from lithium, sodium, potassium, magnesium, calcium, aluminium and zinc, and organic salts made from organic bases such as choline, diethanolamine and morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts, such as tetramethylammonium salt, amino acid addition salts, such as salts with glycine and arginine.

The compounds of formula (I) also extend to include all derivatives with physiologically cleavable leaving groups that can be cleaved in vivo to provide the compounds of formula (I).

Compounds of the formula (I) may be synthesised as described in the Examples section below. Armed with these synthetic procedures and the common general knowledge, those skilled in the art will readily be able to prepare all compounds embraced by formula (I).

After purification, compounds of formula (I) may be substantially pure. For example, the compounds of formula (I) may be isolated in a form which is at least about 80%, 81%, 82%, 83%, 84%, 85%, 86%, 87%, 88%, 89%, 90%, 91%, 92%, 93%, 94%, 95%, 96%, 97%, 98%, 99%, 99.5% or 99.9% pure.

Compounds of the formula (I) may be obtained as racemic mixtures. Enantiomers may be isolated using techniques known to those skilled in the art, including chiral resolution, supercritical fluid chromatography and enantioselective syntheses. Individual enantiomers may be isolated in a substantially pure form or in an enantiomeric excess (ee). For example, in preferred embodiments an enantiomer may be isolated in an enantiomeric excess of about 70%, 75%, 80%, 85%, 90%, 91%, 92%, 93%, 94%, 95%, 96%, 97%, 98%, 99% or greater than 99%.

The compounds of formula (I) find use in the treatment of cancer. The compounds of formula (I) may be used in conjunction with, or alternatively in the absence of, other chemotherapeutic agents. In some embodiments the compounds of formula (I) may be used in the treatment of cancer that is resistant to one or more chemotherapeutic agents.

The compounds of formula (I) may find use in treating cancer that has recurred in a subject and in reducing the incidence of, or the risk of, recurrence of cancer in a subject deemed to be at risk of cancer recurrence, for example a subject who is in cancer remission. Compounds of the formula (I) may also find use in preventing or slowing cancer spread, such as for example, preventing or slowing metastasis.

The cancer may be a solid tumour, such as for example, neuroblastoma, sarcoma, breast cancer, lung cancer, prostate cancer, ovarian cancer, bone cancer, uterine cancer, peritoneal cancer, brain cancer, skin cancer, colon cancer, testicular cancer, colorectal cancer, cervical cancer, renal cancer, bladder cancer, gastric cancer, pancreatic cancer, gall bladder cancer, liver cancer, pancreatic cancer, head and neck cancer, throat cancer and esophageal cancer.

In some embodiments the cancer is lung cancer, ovarian cancer or neuroblastoma. In one embodiment the lung cancer may be non-small cell lung cancer.

Those skilled in the art will recognise that compounds and pharmaceutical compositions of the disclosure may be administered via any route which delivers an effective amount of the compounds to the tissue or site to be treated. In general, the compounds and compositions may be administered by the parenteral (for example intravenous, intraspinal, subcutaneous or intramuscular), oral, rectal or topical route. Administration may be systemic, regional or local.

The particular route of administration to be used in any given circumstance will depend on a number of factors, including the nature of the cancer to be treated, the severity and extent of the cancer, the required dosage of the particular compound to be delivered and the potential side-effects of the compound.

In general, suitable compositions may be prepared according to methods that are known to those of ordinary skill in the art and may include pharmaceutically acceptable carriers, diluents and/or excipients. The carriers, diluents and excipients must be “acceptable” in terms of being compatible with the other ingredients of the composition, and not deleterious to the recipient thereof.

Examples of pharmaceutically acceptable carriers or diluents are demineraiised or distilled water; saline solution; vegetable-based oils such as peanut oil, safflower oil, olive oil, cottonseed oil, maize oil or coconut oil; silicone oils, including polysiloxanes, such as methyl polysiloxane, phenyl polysiloxane and methylphenyl polysiloxane: volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; Cremophor®; cyclodextrins; lower alcohols, for example ethanol or i-propanol; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia and petroleum jelly. Typically, the carrier or carriers will form from about 10% to about 99.9% by weight of the compositions.

Pharmaceutical compositions may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include cyclodextrins (for example Captisol®) Cremophor®, Ringer's solution, isotonic saline, phosphate buffered saline, ethanol and 1,2 propylene glycol. To aid injection and delivery, the compounds may also be added to PEG and non-PEGylated liposomes or micelles with specific targeting tags attached to PEG moieties, such as the RGD peptide or glutathione, for aiding passage across the blood brain barrier.

Some examples of suitable carriers, diluents, excipients and adjuvants for oral use include cyclodextrins, Cremophor®, peanut oil, liquid paraffin, sodium carboxymethylcellulose. methylcellulose, sodium alginate, gum acacia, gum tragacanth, dextrose, sucrose, sorbitol, mannitol, gelatine and lecithin. In addition, these oral formulations may contain suitable flavouring and colourings agents. When used in capsule form, the capsules may be coated with compounds such as glyceryl monostearate or glyceryl distearate that delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose, or polyethylene glycol. Suitable sweeteners include sucrose, lactose, glucose, aspartame or saccharin. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms suitable for oral administration may contain, in addition to the above agents, a liquid carrier. Suitable liquid carriers include water, oils such as olive oil, peanut oil, sesame oil, sunflower oil, safflower oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, PA, the entirety of which is hereby incorporated by reference.

Topical formulations may comprise an active ingredient together with one or more acceptable carriers, and optionally any other therapeutic ingredients. Formulations suitable for topical administration include liquid or semi-liquid preparations suitable for penetration through the skin to the site where treatment is required, such as liniments, lotions, creams, ointments or pastes, and drops suitable for administration to the eye, ear or nose.

Drops may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by autoclaving or maintaining at 90° C. to 100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as olive oil.

Creams, ointments or pastes are typically semi-solid formulations of the active ingredient for external application. They may be made by mixing the active ingredient in finely divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol, such as propylene glycol or macrogols.

The composition may incorporate any suitable surfactant such as an anionic, cationic or non-ionic surfactant, such as sorbitan esters or polyoxyethylene derivatives thereof. Suspending agents such as natural gums, cellulose derivatives or inorganic materials such as silicaceous silicas, and other ingredients such a lanolin, may also be included.

In some embodiments the compositions are administered in the form of suppositories suitable for rectal administration of the compounds of formula (I). These compositions are prepared by mixing the compound of formula (I) with a suitable non-irritating excipient which is solid at ordinary temperatures but liquid at the rectal temperature and will therefore melt in the rectum to release the compound of formula (I). Such materials include cocoa butter, glycerinated gelatin, hydrogenated vegetable oils, mixtures of polyethylene glycols of various molecular weights and fatty acid esters of polyethylene glycol.

The compositions may also be administered or delivered to target cells in the form of liposomes. Liposomes are generally derived from phospholipids or other lipid substances and are formed by mono- or multi-lamellar hydrated liquid crystals that are dispersed in an aqueous medium. Specific examples of liposomes used in administering or delivering a composition to target cells are synthetic cholesterol (Sigma), the phospholipid 1,2-distearoyl-sn-glycero-3-phosphocholine (DSPC, Avanti Polar Lipids), the PEG lipid 3-N-[(-methoxy poly(ethylene glycol)2000)carbamoyl]-1,2-dimyrestyloxy-propylamine (PEG-cDMA), and the cationic lipid 1,2-di-o-octadecenyl-3-(N.N-dimethyl)aminopropane (DODMA) or 1,2-dilinoleyloxy-3-(N,N-dimethylaminopropane (DLinDMA) in the molar ratios 55:20:10:15 or 48:20:2:30, respectively, PEG-cDMA, DODMA and DLinDMA. The liposome may be constructed from 1,2-distearoyl-sn-glycero-3-phosphoethanoiamine-N-[methoxy(polyethyleneglycol)-2000] (DSPE PEG2000) and phosphatidylcholine derived from soy and hydrogenated between 50-100%, for example Soy PC-75 or Soy PC-100. Differing MW PEG's may be used and covalently bound with various specific targeting agents such as glutathione, RGD peptides or other recognized liposome targeting agents. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this, specific reference is made to: Prescott, Ed., Methods in Cell Biology, Volume XIV, Academic Press, New York, N Y. (1976), p. 33 et seq., the contents of which is incorporated herein by reference.

The compositions may also be administered in the form of microparticles or nanoparticles. Biodegradable microparticles formed from polylactide (PLA), polylactide-co-glycolide (PLGA), and epsilon-caprolactone (\acute{ε}-caprolactone) have been extensively used as drug carriers to increase plasma half-life and thereby prolong efficacy (R. Kumar, M. 2000. J. Pharm. Pharmaceut. Sci. 3(2) 234-258). Microparticles have been formulated for the delivery of a range of drug candidates including vaccines, antibiotics, and DNA. Moreover, these formulations have been developed for various delivery routes including parenteral subcutaneous injection, intravenous injection and inhalation.

The compositions may incorporate a controlled release matrix that is composed of sucrose acetate isobutyrate (SAIB) and an organic solvent or organic solvents mixture. Polymer additives may be added to the vehicle as a release modifier to further increase the viscosity and slow down the release rate. SAIB is a well-known food additive. It is a very hydrophobic, fully esterified sucrose derivative, at a nominal ratio of six isobutyrate to two acetate groups. As a mixed ester, SAIB does not crystallise but rather exists as a clear viscous liquid. Mixing SAIB with a pharmaceutically acceptable organic solvent, such as ethanol or benzyl alcohol decreases the viscosity of the mixture sufficiently to allow for injection. An active pharmaceutical ingredient may be added to the SAIB delivery vehicle to form SAIB solution or suspension formulations. When the formulation is injected subcutaneously. the solvent differs from the matrix allowing the SAIB-drug or SAIB-drug-polymer mixtures to set up as an in situ forming depot.

For the purposes of the present disclosure, compounds and compositions may be administered to subjects either therapeutically or preventively. In a therapeutic application compositions are administered to a patient already suffering from cancer in an amount sufficient to cure, or at least partially arrest the cancer and its complications. The composition should provide a quantity of the compound sufficient to effectively treat the subject.

The therapeutically effective amount for any particular subject will depend upon a variety of factors including: the cancer being treated and the severity thereof; the activity of the compound administered; the composition in which the compound is present; the age, body weight, general health, sex and diet of the subject; the time of administration; the route of administration; the rate of sequestration of the compound; the duration of the treatment; drugs used in combination or coincidental with the compound, together with other related factors well known in medicine.

One skilled in the art would be able, by routine experimentation, to determine an effective, non-toxic amount of a compound that would be required to treat or prevent a particular cancer.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the cancer.

Further, it will be apparent to one of ordinary skill in the art that the optimal quantity and spacing of individual dosages will be determined by the nature and extent of the cancer being treated, the form, route and site of administration, and the nature of the particular individual being treated. Also, such optimum conditions can be determined by conventional techniques.

The compounds of formula (I) may be used alone in the treatment of cancer, or alternatively in combination with radiotherapy and/or surgery and/or other therapeutic agents, for example chemotherapeutic agents and immunostimulatory agents, as part of a combination therapy. The compounds of formula (I) may sensitise cancer cells to other chemotherapeutic agents and/or radiotherapy.

In some embodiments the compounds of formula (I) may be administered in combination with anti-microtubule agents as a combination therapy. Anti-microtubule agents interfere with microtubule function, particularly within the mitotic spindle and include the vinca alkaloids (such as for example vincristine, vinorelbine, vinblastine and the like) and the taxanes (such as for example paclitaxel and docetaxel).

The terms “combination therapy” and “adjunct therapy” are intended to embrace administration of multiple therapeutic agents in a sequential manner in a regimen that will provide beneficial effects and is intended to embrace administration of these agents in either a single formulation or in separate formulations.

Combination therapy may involve the active agents being administered together, sequentially, or spaced apart as appropriate in each case. Combinations of active agents including compounds of the disclosure may be synergistic.

The co-administration of compounds of the formula (I) with other therapeutic agent(s) may be effected by a compound of the formula (I) being in the same unit dose form as the other therapeutic agent(s), or the compound of the formula (I) and the other therapeutic agent(s) may be present in individual and discrete unit dosage forms that are administered sequentially, at the same, or at a similar time. Sequential administration may be in any order as required, and may require an ongoing physiological effect of the first or initial agent to be current when the second or later agent is administered, especially where a cumulative or synergistic effect is desired. When administered separately, it may be preferred for the compound of formula (I) and the other agent to be administered by the same route of administration, although it is not necessary for this to be so.

In accordance with various embodiments of the present disclosure one or more compounds of formula (I) may be included in combination therapy with surgery and/or radiotherapy and/or one or more chemotherapeutic agents.

There are large numbers of chemotherapeutic agents that are currently in use, in clinical evaluation and in pre-clinical development, which could be selected for treatment of cancers in combination with compounds of the formula (I).

### EXAMPLES

The present disclosure is further described below by reference to the following non-limiting examples.

### Example 1—Synthesis of Compounds of Formula (I)

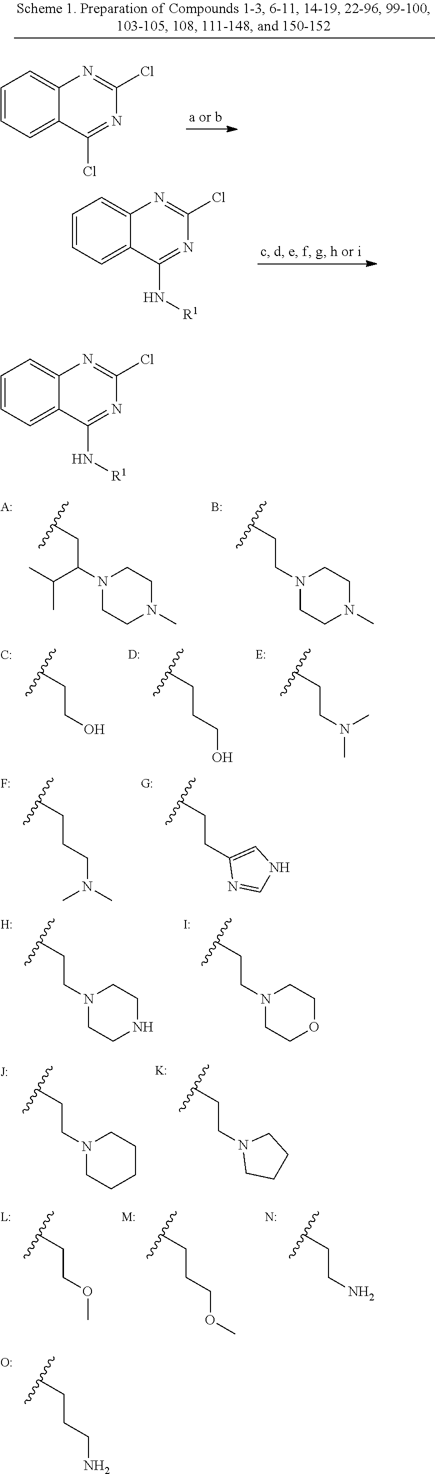

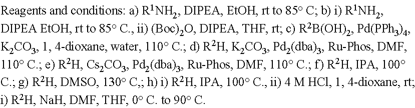

TABLE 1
Compounds prepared according to Scheme 1

| Compound | R¹ | R² | Step 2 Conditions |
|---|---|---|---|
| 1 | A | 3-thiophenyl | c |
| 2 | C | NHCH₂(3-CF₃)Ph | d |
| 3 | A | 2-thiophenyl | c |
| 6 | A | 2-furanyl | c |
| 7 | A | 3-furanyl | c |
| 8 | A | N-pyrrolidinyl | f |
| 9 | A | N-(1H-pyrrolyl) | e |
| 10 | B | 2-thiophenyl | c |
| 11 | B | 3-thiophenyl | c |
| 14 | B | 2-furanyl | c |
| 15 | B | 3-furanyl | c |
| 16 | B | N-pyrrolidinyl | f |
| 17 | B | N-(1H-pyrrolyl) | e |
| 18 | C | 2-thiophenyl | c |
| 19 | C | 3-thiophenyl | c |
| 22 | C | 2-furanyl | c |
| 23 | C | 3-furanyl | c |
| 24 | C | N-pyrrolidinyl | d |
| 25 | C | N-(1H-pyrrolyl) | d |
| 26 | C | NHPh | d |
| 27 | C | NH(3-CF₃—Ph) | d |
| 28 | C | NH(4-CF₃—Ph) | d |
| 29 | C | NH(3-OMe—Ph) | d |
| 30 | C | NH(4-OMe—Ph) | d |
| 31 | C | NHCH₂Ph | d |
| 32 | C | NHCH₂(4-CF₃—Ph) | d |
| 33 | C | NHCH₂(3-OMe—Ph) | d |
| 34 | C | NHCH₂(4-OMe—Ph) | d |
| 35 | C | NH(CH₂)₂Ph | d |
| 36 | C | NH(CH₂)₂(3-CF₃—Ph) | d |
| 37 | C | NH(CH₂)₂(4-CF₃—Ph) | d |
| 38 | C | NH(CH₂)₂(3-OMe—Ph) | d |
| 39 | C | NH(CH₂)₂(4-OMe—Ph) | d |
| 40 | B | NHPh | e |
| 41 | B | NH(3-CF₃—Ph) | e |
| 42 | B | NH(4-CF₃—Ph) | e |
| 43 | B | NH(3-OMe—Ph) | e |
| 44 | B | NH(4-OMe—Ph) | d |
| 45 | B | NHCH₂Ph | f |
| 46 | B | NHCH₂(3-CF₃—Ph) | f |
| 47 | B | NHCH₂(4-CF₃—Ph) | f |
| 48 | B | NHCH₂(3-OMe—Ph) | f |
| 49 | B | NHCH₂(4-OMe—Ph) | f |
| 50 | B | NH(CH₂)₂Ph | f |
| 51 | B | NH(CH₂)₂(3-CF₃—Ph) | f |
| 52 | B | NH(CH₂)₂(4-CF₃—Ph) | f |
| 53 | B | NH(CH₂)₂(3-OMe—Ph) | f |
| 54 | B | NH(CH₂)₂(4-OMe—Ph) | f |
| 55 | B | NH(4-Et—Ph) | d |
| 56 | B | NH(3-F—Ph) | d |
| 57 | B | NH(CH₂)₂(4-Et—Ph) | f |
| 58 | B | NH(CH₂)₂(3-F—Ph) | f |
| 59 | B | NH(CH₂)₂(4-F—Ph) | f |
| 60 | E | NH(3-CF₃—Ph) | d |
| 61 | E | NH(4-OMe—Ph) | d |
| 62 | E | NH(4-Et—Ph) | f |
| 63 | E | NH(3-F—Ph) | f |
| 64 | E | NH(CH₂)₂(3-CF₃—Ph) | e |
| 65 | E | NH(CH₂)₂(4-OMe—Ph) | e |
| 66 | E | NH(CH₂)₂(4-Et—Ph) | e |
| 67 | E | NH(CH₂)₂(3-F—Ph) | f |
| 68 | E | NH(CH₂)₂(4-F—Ph) | f |
| 69 | F | NH(3-CF₃—Ph) | e |
| 70 | F | NH(4-OMe—Ph) | e |
| 71 | F | NH(4-Et—Ph) | e |
| 72 | F | NH(3-F—Ph) | e |
| 73 | F | NH(4-F—Ph) | e |
| 74 | F | NH(CH₂)₂(3-CF₃—Ph) | g |
| 75 | F | NH(CH₂)₂(4-OMe—Ph) | f |
| 76 | F | NH(CH₂)₂(4-Et—Ph) | f |
| 77 | F | NH(CH₂)₂(3-F—Ph) | f |
| 78 | F | NH(CH₂)₂(4-F—Ph) | g |
| 79 | G | NH(3-CF₃—Ph) | d |
| 80 | G | NH(4-OMe—Ph) | d |
| 81 | G | NH(4-Et—Ph) | d |
| 82 | G | NH(3-F—Ph) | d |
| 83 | G | NH(4-F—Ph) | d |
| 84 | G | NH(CH₂)₂(3-CF₃—Ph) | g |
| 85 | G | NH(CH₂)₂(4-OMe—Ph) | g |
| 86 | G | NH(CH₂)₂(4-Et—Ph) | g |
| 87 | G | NH(CH₂)₂(3-F—Ph) | g |
| 88 | G | NH(CH₂)₂(4-F—Ph) | g |
| 89 | C | NH(CH₂)₂(4-Et—Ph) | f |
| 90 | C | NH(CH₂)₂(3-F—Ph) | f |
| 91 | C | NH(CH₂)₂(4-F—Ph) | f |
| 92 | D | NH(CH₂)₂(3-CF₃—Ph) | f |
| 93 | D | NH(CH₂)₂(4-Et—Ph) | f |
| 94 | D | NH(CH₂)₂(3-F—Ph) | f |
| 95 | D | NH(CH₂)₂(4-F—Ph) | f |
| 96 | E | 3-thiophenyl | c |
| 99 | E | N-(1H-pyrrolyl) | d |
| 100 | F | 3-thiophenyl | c |
| 103 | F | N-(1H-pyrrolyl) | d |
| 104 | G | 2-thiophenyl | c |
| 105 | G | 3-thiophenyl | c |
| 108 | G | N-(1H-pyrrolyl) | d |
| 111 | D | N-(1H-pyrrolyl) | d |
| 112 | H | NH(CH₂)₂(3-CF₃—Ph) | h |
| 113 | I | NH(CH₂)₂(3-CF₃—Ph) | f |
| 114 | J | NH(CH₂)₂(3-CF₃—Ph) | f |
| 115 | K | NH(CH₂)₂(3-CF₃—Ph) | f |
| 116 | L | NH(CH₂)₂(3-CF₃—Ph) | f |
| 117 | M | NH(CH₂)₂(3-CF₃—Ph) | f |
| 118 | N | NH(CH₂)₂(3-CF₃—Ph) | h |
| 119 | O | NH(CH₂)₂(3-CF₃—Ph) | h |
| 120 | B | NH(CH₂)₂(2-CF₃—Ph) | f |
| 121 | B | NH(CH₂)₂(2-F—Ph) | f |
| 122 | B | NH(CH₂)₂(2-Me—Ph) | f |
| 123 | B | NH(CH₂)₂(2-Cl—Ph) | f |
| 124 | B | NH(CH₂)₂(3-Me—Ph) | f |
| 125 | B | NH(CH₂)₂(3-Cl—Ph) | f |
| 126 | B | NH(CH₂)₂(3-CN—Ph) | f |
| 127 | B | NH(CH₂)₂(4-Me—Ph) | f |
| 128 | B | NH(CH₂)₂(4-iPr—Ph) | f |
| 129 | B | NH(CH₂)₂(4-tBu—Ph) | f |
| 130 | B | NH(CH₂)₂(4-Cl—Ph) | f |
| 131 | B | NH(CH₂)₂(4-CN—Ph) | f |
| 132 | B | NH(CH₂)₂(4-OH—Ph) | f |
| 133 | B | NH(CH₂)₂(3-NH₂—Ph) | f |
| 134 | B | NH(CH₂)₂(3-NMe₂—Ph) | f |
| 135 | B | NH(CH₂)₂(3,4-diCl—Ph) | f |
| 136 | B | NH(CH₂)₂(3,4-diF—Ph) | f |
| 137 | B | NH(CH₂)₂(3,4-diOMe—Ph) | f |
| 138 | B | NH(CH₂)₂(3,5-diCl—Ph) | f |
| 139 | B | NH(CH₂)₂(3,5-diF—Ph) | f |
| 140 | B | NH(CH₂)₂(3,5-diOMe—Ph) | f |
| 141 | B | NH(CH₂)₂(3,4,5-triF—Ph) | f |
| 142 | B | NH(CH₂)₂(3,4,5-triOMe—Ph) | f |
| 143 | B | NH(CH₂)₂(3,4-methylendioxy-Ph) | f |
| 144 | B | NH(CH₂)₂(2-Py) | f |
| 145 | B | NH(CH₂)₂(3-Py) | f |
| 146 | B | NH(CH₂)₂(4-Py) | f |
| 147 | B | NH(CH₂)₂(2-thiophenyl) | f |
| 148 | B | NH(CH₂)₂(2-furyl) | f |
| 150 | B | O(CH₂)₂(3-CF₃—Ph) | i |
| 151 | B | S(CH₂)₂(3-CF₃—Ph) | i |
| 152 | B | NH(CH₂)₂(1-Me-1H-pyrrol-2-yl) | f |

### Preparation of 2-((2-chloroquinazolin-4-yl)amino) ethan-1-ol

To a stirred solution of 2,4-dichloroquinazoline (1.00 g, 5.02 mmol) in ethanol (10 mL) was added DIPEA (1.75 mL, 10.04 mmol) and 2-aminoethan-1-ol (368 mg, 6.02 mmol) at room temperature. The mixture was then heated to 8500 and stirred for 5 h. The progress of the reaction was monitored by TLC. After complete consumption of the starting material, the reaction mixture was concentrated under reduced pressure. The crude product thus obtained was then purified by column chromatography using 100-200 mesh silica gel to obtain the title compound as an off-white solid (750 mg, 67%). LCMS: m/z 224.16 [M+H]⁺.

Other analogues prepared by this method:

2-chloro-N-(3-methyl-2-(4-methylpiperazin-1-yl)butyl)quinazolin-4-amine (80%). LCMS: m/z 348.38 [M+H]⁺.

2-chloro-N-(2-(4-methylpiperazin-1-yl)ethyl)quinazolin-4-amine (62%). LCMS: m/z 306.31 [M+H]⁺.

3-((2-chloroquinazolin-4-yl)amino)propan-1-ol (55%).

¹H NMR (400 MHz, DMSO-d₆): δ 8.70 (t, J=5.6 Hz, 1H), 8.60 (d, J=8.0 Hz, 1H), 7.78 (t, J=7.6 Hz, 1H), 7.58 (d, J=7.6 Hz, 1H), 7.51 (t, J=8.0 Hz, 1H), 4.55 (t, J=5.2 Hz, 1H), 3.58-3.49 (m, 4H), 1.85-1.79 (m, 2H).

### N¹-(2-chloroquinazolin-4-yl)-N²,N²-dimethylethane-1,2-diamine (48%)

¹H NMR (400 MHz, DMSO-d₆): δ 8.67 (br s, 1H), 8.25 (d, J=8.0 Hz, 1H), 7.79 (t, J=8.0 Hz, 1H), 7.62 (d, J=7.6 Hz, 1H), 7.53 (t, J=7.6 Hz, 1H), 3.62 (t, J=6.0 Hz, 2H), 2.54-2.50 (m, 2H), 2.21 (s, 6H).

N¹-(2-chloroquinazolin-4-yl)-N³,N³-diimethylpropane-1,3-diamine (56%). LCMS: m/z 263.55 [M−H]⁻.

N-(2-(1H-imidazol-4-yl)ethyl)-2-chloroquinazolin-4-amine (51%).

¹H NMR (400 MHz, DMSO-d₆): δ 11.90 (br s, 1H), 8.83 (br s, 1H), 8.23 (d, J=8.0 Hz, 1H), 7.79 (t, J=8.0 Hz, 1H), 7.70-7.48 (m, 3H), 6.86 (br s, 1H), 3.77-3.71 (m, 2H), 2.89 (t, J=7.6 Hz, 2H).

tert-butyl 4-(2-((2-chloroquinazolin-4-yl)amino)ethyl)piperazine-1-carboxylate (61%). LCMS: m/z 390.40 [M+H]⁺.

2-chloro-N-(2-morpholinoethyl)quinazolin-4-amine (54%). LCMS: m/z 293.15 [M+H]⁺.

2-chloro-N-(2-(piperidin-1-yl)ethyl)quinazolin-4-amine (43%). LCMS: m/z 291.18 [M+H]⁺.

2-chloro-N-(2-(pyrrolidin-1-yl)ethyl)quinazolin-4-amine (37%). LCMS: m/z 277.17 [M+H]⁺.

2-chloro-N-(2-methoxyethyl)quinazolin-4-amine (59%). LCMS: m/z 238.23 [M+H]⁺.

2-chloro-N-(3-methoxypropyl)quinazolin-4-amine (44%). LCMS: m/z 252.21 [M+H]⁺.

N¹-(2-chloroquinazolin-4-yl)ethane-1,2-diamine (63%). LCMS: m/z 223.24 [M+H]⁺.

N¹-(2-chloroquinazolin-4-yl)propane-1,3-diamine (46%). LCMS: m/z 237.22 [M+H]⁺.

### Preparation of Tert-Butyl (2-((2-chloroquinazolin-4-yl)amino)ethyl)carbamate

To a stirred solution of N¹-(2-chloroquinazolin-4-yl)ethane-1,2-diamine (700 mg, 3.14 mmol) in THF (10 mL), was added DIPEA (0.60 mL, 3.4 mmol), and Boc anhydride (740 mg, 3.4 mmol) at 0° C. The resulting mixture was then stirred at room temperature for 2 h. The progress of the reaction was monitored by TLC. After complete consumption of the starting material, the reaction mixture was quenched with ice water and extracted with EtOAc (2×20 mL). The combined organic layer was dried over anhydrous Na₂SO₄ and concentrated under reduced pressure. The crude product thus obtained was then purified by column chromatography using 100-200 mesh silica gel to obtain the title compound as an off-white solid (500 mg, 49%). LCMS: m/z 323.31 [M+H]⁺.

Other analogues prepared by this method:

tert-butyl (3-((2-chloroquinazolin-4-yl)amino)propyl)carbamate (77%). LCMS: m/z 337.34 [M+H]⁺.

### Preparation of Compound 1, N-(3-methyl-2-(4-methylpiperazin-1-yl)butyl)-2-(thiophen-3-yl)quinazolin-4-amine

To a stirred solution of 2-chloro-N-(3-methyl-2-(4-methylpiperazin-1-yl)butyl)quinazolin-4-amine (100 mg, 0.29 mmol) and thiophen-3-yl boronic acid (45 mg, 0.35 mmol) in 1,4-dioxane (15 mL) and H₂O (0.5 mL) was added K₂CO₃ (120 mg, 0.87 mmol) at room temperature. The reaction mixture was degassed with argon for 20 min, after which was added Pd(PPh₃)₄ (35 mg, 0.03 mmol) at room temperature. The reaction mixture was heated to 110° C. and stirred at that temperature for 16 h. The progress of the reaction was monitored by TLC. After complete consumption of the starting material, the reaction mixture was quenched with H₂O (30 mL) and extracted with EtOAc (2×50 mL). The combined organic layer was dried over anhydrous Na₂SO₄ and concentrated under reduced pressure to give the crude product. The crude compound was purified by preparative HPLC to obtain the title compound as an off-white solid (75 mg, 66%).

¹H NMR (400 MHz, DMSO-d₆): δ 8.30 (dd, J=3.2 Hz, 0.8 Hz, 1H), 8.21 (d, J=8.0 Hz, 1H), 8.03 (t, J=5.2 Hz, 1H), 7.87 (dd, J=5.2 Hz, 0.8 Hz, 1H), 7.76-7.68 (m, 2H), 7.62-7.59 (m, 1H), 7.49-7.44 (m, 1H), 3.79-3.75 (m, 2H), 2.77-2.60 (m, 5H), 2.18 (br s, 4H), 2.06 (s, 3H), 1.86-1.77 (m, 1H), 1.05 (d, J=6.4 Hz, 3H), 0.98 (d, J=6.8 Hz, 3H). LCMS: m/z 396.1 [M+H]⁺.

Other analogues prepared by this method:

### Compound 3, N-(3-methyl-2-(4-methylpiperazin-1-yl)butyl)-2-(thiophen-2-yl)quinazolin-4-amine (38%)

¹H NMR (400 MHz, DMSO-d₆): δ 8.21 (d, J=8.4 Hz, 1H), 8.13 (t, J=5.4 Hz, 1H), 7.92 (dd, J=3.6 Hz, 1.2 Hz, 1H), 7.75-7.72 (m, 1H), 7.68-7.62 (m, 2H), 7.48-7.44 (m, 1H), 7.18 (dd, J=8.8 Hz, 3.6 Hz, 1H), 3.79-3.71 (m, 2H), 2.79-2.61 (m, 5H), 2.18 (br s, 4H), 2.06 (s, 3H), 1.83-1.78 (m, 1H), 1.06 (d, J=6.4 Hz, 3H), 0.98 (d, J=6.8 Hz, 3H). LCMS: m/z 396.41 [M+H]⁺.

### Compound 6, 2-(furan-2-yl)-N-(3-methyl-2-(4-methylpiperazin-1-yl)butyl)quinazolin-4-amine (84%)

¹H NMR (400 MHz, DMSO-d₆): δ 8.20 (d, J=8.0 Hz, 1H), 8.08 (t, J=5.2 Hz, 1H), 7.85 (s, 1H), 7.76-7.68 (m, 2H), 7.49-7.45 (m, 1H), 7.21 (d, J=3.6 Hz, 1H), 6.66 (dd, J=3.2 Hz, 2.0 Hz, 1H), 3.75-3.72 (m, 2H), 2.74-2.69 (m, 5H), 2.20 (br s, 4H), 2.07 (s, 3H), 1.82-1.75 (m, 1H), 1.04 (d, J=6.4 Hz, 3H), 0.98 (d, J=6.8 Hz, 3H). LCMS: m/z 380.35 [M+H]⁺.

### Compound 7, 2-(furan-3-yl)-N-(3-methyl-2-(4-methylpiperazin-1-yl)butyl)quinazolin-4-amine (66%)

¹H NMR (400 MHz, DMSO-d₆): δ 8.31 (d, J=0.8 Hz, 1H), 8.19 (d, J=8.0 Hz, 1H), 8.04 (t, J=5.2 Hz, 1H), 7.77-7.75 (m, 2H), 7.67 (d, J=7.6 Hz, 1H), 7.46 (t, J=8.0 Hz, 1H), 7.05 (d, J=1.2 Hz, 1H), 3.82-3.69 (m, 2H), 2.75-2.67 (m, 5H), 2.42-2.07 (m, 7H), 1.85-1.76 (m, 1H), 1.04 (d, J=6.4 Hz, 3H), 0.98 (d, J=6.8 Hz, 3H). LCMS: m/z 380.36 [M+H]⁺.

### Compound 10, N-(2-(4-methylpiperazin-1-yl)ethyl)-2-(thiophen-2-yl)quinazolin-4-amine (22%)

¹H NMR (400 MHz, DMSO-d₆): δ 8.30 (t, J=5.6 Hz, 1H), 8.17 (d, J=7.6 Hz, 1H), 7.91 (dd, J=3.6 Hz, 1.2 Hz, 1H), 7.80-7.66 (m, 3H), 7.47-7.45 (m, 1H), 7.18 (dd, J=4.8 Hz, 3.6 Hz, 1H), 3.74 (q, J=6.4 Hz, 2H), 2.64 (t, J=7.2 Hz, 2H), 2.54-2.53 (m, 4H), 2.33-2.35 (m, 4H), 2.13 (s, 3H). LCMS: m/z 352.30 [M−H]⁻.

### Compound 11, N-(2-(4-methylpiperazin-1-yl)ethyl)-2-(thiophen-3-yl)quinazolin-4-amine (35%)

¹H NMR (400 MHz, DMSO-d₆): δ 8.30 (dd, J=3.2 Hz, 1.2 Hz, 1H), 8.22-8.17 (m, 2H), 7.86 (dd, J=5.2, 1.2 Hz, 1H), 7.76-7.68 (m, 2H), 7.60 (dd, J=4.8 Hz, 3.2 Hz, 1H), 7.47-7.43 (m, 1H), 3.76 (q, J=6.4 Hz, 2H), 2.64 (t, J=7.2 Hz, 2H), 2.51-2.49 (m, 4H), 2.33-2.32 (m, 4H), 2.14 (s, 3H). LCMS: m/z 354.32 [M+H]⁺.

### Compound 14, 2-(furan-2-yl)-N-(2-(4-methylpiperazin-1-yl)ethyl)quinazolin-4-amine (45%)

¹H NMR (400 MHz, DMSO-d₆): δ 8.24 (t, J=5.6 Hz, 1H), 8.17 (d, J=7.6 Hz, 1H), 7.85 (dd, J=1.6 Hz, 0.8 Hz, 1H), 7.76-7.74 (m, 1H), 7.70-7.68 (m, 1H), 7.48-7.44 (m, 1H), 7.21 (dd, J=3.2 Hz, 0.8 Hz, 1H), 6.66 (dd, J=3.2 Hz, 1.6 Hz, 1H), 3.73 (q, J=6.4 Hz, 2H), 2.65-2.51 (m, 6H), 2.49-2.32 (m, 4H), 2.19 (s, 3H). LCMS: m/z 338.31 [M+H]⁺.

### Compound 15, 2-(furan-3-yl)-N-(2-(4-methylpiperazin-1-yl)ethyl)quinazolin-4-amine (35%)

¹H NMR (400 MHz, DMSO-d₆): δ 8.32 (d, J=0.8 Hz, 1H), 8.21-8.15 (m, 2H), 7.76-7.75 (m, 2H), 7.67 (dd, J=8.4 Hz, 0.8 Hz, 1H), 7.46-7.42 (m, 1H), 7.04 (dd, J=2.0 Hz, 0.8 Hz, 1H), 3.74 (q, J=6.4 Hz, 2H), 2.65-2.49 (m, 10H), 2.29 (s, 3H). LCMS: m/z 338.35 [M+H]⁺.

### Compound 18, 2-((2-(thiophen-2-yl)quinazolin-4-yl)amino)ethan-1-ol (11%)

¹H NMR (400 MHz, DMSO-d₆): δ 8.34 (br s, 1H), 8.23 (d, J=8.0 Hz, 1H), 7.93 (d, J=2.4 Hz, 1H), 7.76-7.72 (m, 1H), 7.68-7.65 (m, 2H), 7.47-7.43 (m, 1H), 7.18 (dd, J=4.8 Hz, 3.6 Hz, 1H), 4.82 (br s, 1H), 3.73-3.69 (m, 4H). LCMS: m/z 272.15 [M+H]⁺.

### Compound 19, 2-((2-(thiophen-3-yl)quinazolin-4-yl)amino)ethan-1-ol (16%)

¹H NMR (400 MHz, DMSO-d₆): δ 8.33 (dd, J=2.8 Hz, 0.8 Hz, 1H), 8.28 (br s, 1H), 8.24 (d, J=8.0 Hz, 1H), 7.87 (dd, J=8.8 Hz, 0.8 Hz, 1H), 7.76-7.69 (m, 2H), 7.61 (dd, J=4.8 Hz, 3.2 Hz, 1H), 7.46-7.43 (m, 1H), 4.83 (t, J=5.2 Hz, 1H), 3.72 (br s, 4H). LCMS: m/z 272.15 [M+H]⁺.

### Compound 22, 2-((2-(furan-2-yl)quinazolin-4-yl)amino)ethan-1-ol (63%)

¹H NMR (400 MHz, DMSO-d₆): δ 8.29 (br s, 1H), 8.23 (dd, J=8.4 Hz, 0.8 Hz, 1H), 7.86 (dd, J=1.6 Hz, 0.8 Hz, 1H), 7.77-7.74 (m, 1H), 7.70-7.68 (m, 1H), 7.48-7.44 (m, 1H), 7.24 (dd, J=3.2 Hz, 0.8 Hz, 1H), 6.66 (dd, J=3.2 Hz, 1.6 Hz, 1H), 4.85 (t, J=5.6 Hz, 1H), 3.72-3.67 (m, 4H). LCMS: m/z 256.19 [M+H]⁺.

### Compound 23, 2-((2-(furan-3-yl)quinazolin-4-yl)amino)ethan-1-ol (26%)

¹H NMR (400 MHz, DMSO-d₆): δ 8.34 (dd, J=1.2 Hz, 0.8 Hz, 1H), 8.24-8.21 (m, 2H), 7.71-7.76 (m, 2H), 7.68-7.65 (m, 1H), 7.46-7.42 (m, 1H), 7.04 (dd, J=1.6 Hz, 0.8 Hz, 1H), 4.81 (t, J=5.6 Hz, 1H), 3.69 (t, J=2.3 Hz, 4H). LCMS: m/z 256.19 [M+H]⁺.

### Compound 96, N¹,N¹-dimethyl-N²-(2-(thiophen-3-yl)quinazolin-4-yl)ethane-1,2-diamine (10%)

¹H NMR (400 MHz, DMSO-d₆): δ 8.31 (d, J=2.4 Hz, 1H), 8.22-8.18 (m, 2H), 7.86 (d, J=5.2 Hz, 1H), 7.76-7.68 (m, 2H), 7.60 (dd, J=4.8 Hz, 2.8 Hz, 1H), 7.45 (t, J=7.2 Hz, 1H), 3.75 (q, J=6.4 Hz, 2H), 2.58 (t, J=6.8 Hz, 2H), 2.24 (s, 6H). LCMS: m/z 299.28 [M+H]⁺.

### Compound 100, N¹,N¹-dimethyl-N³-(2-(thiophen-3-yl)quinazolin-4-yl)propane-1,3-diamine (10%)

¹H NMR (400 MHz, DMSO-d₆): δ 8.35 (t, J=5.6 Hz, 1H), 8.31 (dd, J=2.8 Hz, 0.8 Hz, 1H), 8.17 (d, J=8.4 Hz, 1H), 7.86 (dd, J=4.8 Hz, 0.8 Hz, 1H), 7.76-7.68 (m, 2H), 7.61 (dd, J=4.8 Hz, 3.2 Hz, 1H), 7.47-7.42 (m, 1H), 3.67 (q, J=6.4 Hz, 2H), 2.36 (t, J=6.8 Hz, 2H), 2.17 (s, 6H), 1.84 (quint, J=6.8 Hz, 2H). LCMS: m/z 313.39 [M+H]⁺.

### Compound 104, N-(2-(1H-imidazol-4-yl)ethyl)-2-(thiophen-2-yl)quinazolin-4-amine (10%)

¹H NMR (400 MHz, DMSO-d₆): δ 11.84 (br s, 1H), 8.47 (br s, 1H), 8.18 (d, J=8.0 Hz, 1H), 7.95 (d, J=2.4 Hz, 1H), 7.76-7.71 (m, 1H), 7.69-7.66 (m, 2H), 7.58 (br s, 1H), 7.47-7.42 (m, 1H), 7.18 (dd, J=4.8 Hz, 3.6 Hz, 1H), 6.95 (br s, 1H), 3.84 (dd, J=12.0 Hz, 7.6 Hz, 2H), 3.01-2.89 (m, 2H). LCMS: m/z 322.29 [M+H]⁺.

### Compound 105, N-(2-(1H-imidazol-4-yl)ethyl)-2-(thiophen-3-yl)quinazolin-4-amine (15%)

¹H NMR (400 MHz, DMSO-d₆): δ 11.79 (br s, 1H), 8.35 (br s, 1H), 8.19 (br d, J=7.6 Hz, 1H), 7.88 (d, J=5.2 Hz, 1H), 7.76-7.69 (m, 2H), 7.61-7.51 (m, 2H), 7.47-7.43 (m, 1H), 6.96 (br s, 1H), 3.85 (dd, J=12.8 Hz, 7.2 Hz, 2H), 3.01-2.90 (m, 2H). LCMS: m/z 322.31 [M+H]⁺.

### Preparation of Compound 2, 2-((2-((3-(trifluoromethyl)benzyl)amino)quinazolin-4-yl)amino)ethan-1-ol

To a stirred solution of 2-((2-chloroquinazolin-4-yl)amino)ethan-1-ol (150 mg, 0.67 mmol) and (3-(trifluoromethyl)phenyl)methanamine (130 mg, 0.74 mmol) in DMF (1.5 mL) was added K₂CO₃ (278 mg, 2.01 mmol) at room temperature. The reaction mixture was degassed with argon for 10 min, after which was added Ru-phos (24 mg, 0.05 mmol) followed by Pd₂(dba)₃ (28 mg, 0.03 mmol) at room temperature. The reaction mixture stirred at 110° C. for 1 h under microwave conditions. The progress of the reaction was monitored by TLC. After complete consumption of the starting material, the reaction mixture was cooled to room temperature, then filtered through a bed of Celite and washed with EtOAc (50 mL). The organic layer was washed with water (30 mL) and extracted with EtOAC (100 mL). The organic layer was dried over anhydrous Na₂SO₄ and concentrated under reduced pressure to obtain the crude product. The crude compound was purified by preparative HPLC to afford the title compound as an off-white solid (55 mg, 23%).

¹H NMR (400 MHz, DMSO-d₆): δ 7.96 (d, J=7.6 Hz, 1H), 7.85 (br s, 1H), 7.72 (br s, 1H), 7.66 (br d, J=6.8 Hz, 1H), 7.57-7.50 (m, 2H), 7.48-7.44 (m, 1H), 7.22-7.15 (m, 2H), 7.02 (t, J=7.2 Hz, 1H), 4.72 (br s, 1H), 4.59 (d, J=6.4 Hz, 2H), 3.56-3.52 (m, 4H). LCMS: m/z 363.21 [M+H]⁺.

Other analogues prepared by this method:

### Compound 24, 2-((2-(pyrrolidin-1-yl)quinazolin-4-yl)amino)ethan-1-ol (40%)

¹H NMR (400 MHz, DMSO-d₆): δ 7.95 (d, J=7.6 Hz, 1H), 7.86 (br s, 1H), 7.48-7.44 (m, 1H), 7.25 (d, J=8.0 Hz, 1H), 7.00 (t, J=7.2 Hz, 1H), 4.75 (t, J=5.2 Hz, 1H), 3.65-3.62 (m, 2H), 3.59-3.50 (m, 6H), 1.91-1.88 (m, 4H). LCMS: m/z 259.21 [M+H]⁺.

### Compound 25, 2-((2-(1H-pyrrol-1-yl)quinazolin-4-yl)amino)ethan-1-ol (19%)

¹H NMR (400 MHz, DMSO-d₆): δ 8.56 (br s, 1H), 8.24 (dd, J=8.4 Hz, 0.8 Hz, 1H), 7.80 (t, J=2.4 Hz, 2H), 7.73 (td, J=8.4 Hz, 1.2 Hz, 1H), 7.62 (dd, J=8.4 Hz, 0.8 Hz, 1H), 7.42-7.38 (m, 1H), 6.26 (t, J=2.4 Hz, 2H), 4.83 (t, J=5.6 Hz, 1H), 3.71-3.70 (m, 4H). LCMS: m/z 255.14 [M+H]⁺.

### Compound 26, 2-((2-(phenylamino)quinazolin-4-yl)amino)ethan-1-ol (16%)

¹H NMR (400 MHz, DMSO-d₆): δ 8.98 (s, 1H), 8.08 (dd, J=8.4 Hz, 0.8 Hz, 1H), 8.03 (t, J=5.2 Hz, 1H), 7.92 (d, J=8.8 Hz, 2H), 7.59-7.55 (m, 1H), 7.39 (dd, J=8.4 Hz, 0.8 Hz, 1H), 7.27-7.23 (m, 2H), 7.18-7.14 (m, 1H), 6.90-6.88 (m, 1H), 4.77 (t, J=5.2 Hz, 1H), 3.71-3.61 (m, 4H). LCMS: m/z 281.22 [M+H]⁺.

### Compound 27, 2-((2-((3-(trifluoromethyl)phenyl)amino)quinazolin-4-yl)amino)ethan-1-ol (20%)

¹H NMR (400 MHz, DMSO-d₆): δ 9.43 (s, 1H), 8.56 (br s, 1H), 8.18-8.12 (m, 2H), 8.04 (br d, J=8.0 Hz, 1H), 7.63-7.59 (m, 1H), 7.46 (t, J=8.0 Hz, 1H), 7.41 (d, J=7.6 Hz, 1H), 7.23-7.18 (m, 2H), 4.78 (t, J=5.2 Hz, 1H), 3.71-3.64 (m, 4H). LCMS: m/z 349.32 [M+H]⁺.

### Compound 28, 2-((2-((4-(trifluoromethyl)phenyl)amino)quinazolin-4-yl)amino)ethan-1-ol (14%)

¹H NMR (400 MHz, DMSO-d₆): δ 9.49 (s, 1H), 8.18-8.11 (m, 4H), 7.64-7.58 (m, 3H), 7.45 (d, J=8.0 Hz, 1H), 7.24-7.20 (m, 1H), 4.79 (t, J=5.2 Hz, 1H), 3.70-3.65 (m, 4H). LCMS: m/z 349.26 [M+H]⁺.

### Compound 29, 2-((2-((3-methoxyphenyl)amino)quinazolin-4-yl)amino)ethan-1-ol (10%)

¹H NMR (400 MHz, DMSO-d₅): δ 8.98 (s, 1H), 8.09-8.04 (m, 2H), 7.78 (t, J=2.0 Hz, 1H), 7.60-7.56 (m, 1H), 7.39 (d, J=8.0 Hz, 2H), 7.19-7.11 (m, 2H), 6.46 (dd, J=8.0 Hz, 2.0 Hz, 1H), 4.77 (t, J=5.2 Hz, 1H), 3.75 (s, 3H), 3.69-3.63 (m, 4H). LCMS: m/z 311.35 [M+H]⁺.

### Compound 30, 2-((2-((4-methoxyphenyl)amino)quinazolin-4-yl)amino)ethan-1-ol (49%)

¹H NMR (400 MHz, DMSO-d₆): δ 8.80 (s, 1H), 8.05 (d, J=7.2 Hz, 1H), 7.97 (t, J=5.2 Hz, 1H), 7.80 (d, J=9.2 Hz, 2H), 7.56-7.52 (m, 1H), 7.34 (d, J=7.6 Hz, 1H), 7.14-7.10 (m, 1H), 6.85 (d, J=9.2 Hz, 2H), 4.76 (t, J=5.2 Hz, 1H), 3.72 (s, 3H), 3.70-3.59 (m, 4H). LCMS: m/z 311.35 [M+H]⁺.

### Compound 31, 2-((2-(benzylamino)quinazolin-4-yl)amino)ethan-1-ol (30%)

¹H NMR (400 MHz, DMSO-d₆): δ 7.95 (d, J=7.6 Hz, 1H), 7.81 (br s, 1H), 7.47-7.43 (m, 1H), 7.35 (d, J=7.6 Hz, 2H), 7.28 (t, J=7.6 Hz, 2H), 7.21-7.16 (m, 2H), 7.10-6.98 (m, 2H), 4.72 (t, J=4.8 Hz, 1H), 4.53 (d, J=6.4 Hz, 2H), 3.58-3.52 (m, 4H). LCMS: m/z 295.27 [M+H]⁺.

### Compound 32, 2-((2-((4-(trifluoromethyl)benzyl)amino)quinazolin-4-yl)amino)ethan-1-ol (12%)

¹H NMR (400 MHz, DMSO-d₆): δ 7.96 (d, J=8.0 Hz, 1H), 7.84 (br s, 1H), 7.65 (d, J=8.4 Hz, 2H), 7.55 (d, J=8.0 Hz, 2H), 7.48-7.43 (m, 1H), 7.21-7.15 (m, 2H), 7.02 (t, J=7.2 Hz, 1H), 4.72 (br s, 1H), 4.60 (d, J=6.4 Hz, 2H), 3.56-3.43 (m, 4H). LCMS: m/z 363.34 [M+H]⁺.

### Compound 33, 2-((2-((3-methoxybenzyl)amino)quinazolin-4-yl)amino)ethan-1-ol (17%)

¹H NMR (400 MHz, DMSO-d₆): δ 7.95 (d, J=7.6 Hz, 1H), 7.81 (br s, 1H), 7.45-7.43 (m, 1H), 7.19 (t, J=8.0 Hz, 2H), 7.03-6.98 (m, 2H), 6.93-6.90 (m, 2H), 6.75 (dd, J=8.0 Hz, 1.2 Hz, 1H), 4.72 (t, J=4.8 Hz, 1H), 4.50 (d, J=6.4 Hz, 2H), 3.71 (s, 3H), 3.58-3.52 (m, 4H). LCMS: m/z 325.29 [M+H]⁺.

### Compound 34, 2-((2-((4-methoxybenzyl)amino)quinazolin-4-yl)amino)ethan-1-ol (18%)

¹H NMR (400 MHz, DMSO-d₆): δ 7.95 (d, J=7.6 Hz, 1H), 7.80 (br s, 1H), 7.48-7.44 (m, 1H), 7.28 (d, J=8.8 Hz, 2H), 7.21 (d, J=8.4 Hz, 1H), 7.02-6.94 (m, 2H), 6.85 (d, J=8.8 Hz, 2H), 4.74 (t, J=5.2 Hz, 1H), 4.45 (d, J=6.4 Hz, 2H), 3.71 (s, 3H), 3.61-3.53 (m, 4H). LCMS: m/z 325.37 [M+H]⁺.

### Compound 35, 2-((2-(phenethylamino)quinazolin-4-yl)amino)ethan-1-ol (44%)

¹H NMR (400 MHz, DMSO-d₆): δ 7.96 (dd, J=8.4 Hz, 0.8 Hz, 1H), 7.80 (brs, 1H), 7.48-7.44 (m, 1H), 7.32-7.17 (m, 6H), 7.01 (t, J=7.2 Hz, 1H), 6.50 (br s, 1H), 4.75 (t, J=5.6 Hz, 1H), 3.64-3.49 (m, 6H), 2.88-2.84 (m, 2H). LCMS: m/z 309.29 [M+H]⁺.

### Compound 36, 2-((2-((3-(trifluoromethyl)phenethyl)amino)quinazolin-4-yl)amino)ethan-1-ol (10%)

¹H NMR (400 MHz, DMSO-d₆): δ 7.96 (d, J=7.2 Hz, 1H), 7.80 (br s, 1H), 7.63 (br s, 1H), 7.59-7.52 (m, 3H), 7.48-7.44 (m, 1H), 7.26-7.10 (m, 1H), 7.01 (t, J=7.2 Hz, 1H), 6.57 (br s, 1H), 4.76 (br s, 1H), 3.63-3.53 (m, 6H), 2.99-2.95 (m, 2H). LCMS: m/z 377.0 [M+H]⁺.

### Compound 37, 2-((2-((4-(trifluoromethyl)phenethyl)amino)quinazolin-4-yl)amino)ethan-1-ol (35%)

¹H NMR (400 MHz, DMSO-d₆): δ 7.96 (dd, J=8.0 Hz, 0.8 Hz, 1H), 7.81 (br s, 1H), 7.65 (d, J=8.0 Hz, 2H), 7.50-7.44 (m, 3H), 7.25-7.23 (m, 1H), 7.01 (t, J=7.2 Hz, 1H), 6.57 (br s, 1H), 4.75 (t, J=5.6 Hz, 1H), 3.64-3.53 (m, 6H), 2.97 (t, J=7.2 Hz, 2H). LCMS: m/z 377.46 [M+H]⁺.

### Compound 38, 2-((2-((3-methoxyphenethyl)amino)quinazolin-4-yl)amino)ethan-1-ol (18%)

¹H NMR (400 MHz, DMSO-d₆): δ 7.96 (dd, J=8.0 Hz, 0.8 Hz, 1H), 7.81 (brs, 1H), 7.48-7.44 (m, 1H), 7.22-7.18 (m, 2H), 7.01 (t, J=7.2 Hz, 1H), 6.84-6.83 (m, 2H), 6.77-6.74 (m, 1H), 6.48 (br s, 1H), 4.76 (br s, 1H), 3.73 (s, 3H), 3.63-3.49 (m, 6H), 2.84 (t, J=7.6 Hz, 2H). LCMS: m/z 339.31 [M+H]⁺.

### Compound 39, 2-((2-((4-methoxyphenethyl)amino)quinazolin-4-yl)amino)ethan-1-ol (13%)

¹H NMR (400 MHz, DMSO-d₆): δ 7.96 (dd, J=8.0 Hz, 0.8 Hz, 1H), 7.80 (brs, 1H), 7.49-7.44 (m, 1H), 7.24-7.17 (m, 3H), 7.01 (t, J=7.2 Hz, 1H), 6.86 (d, J=8.8 Hz, 2H), 6.45 (br s, 1H), 4.77 (t, J=4.8 Hz, 1H), 3.73 (s, 3H), 3.64-3.56 (m, 4H), 3.50-3.44 (m, 2H), 2.80 (t, J=8.0 Hz, 2H). LCMS: m/z 339.32 [M+H]⁺.

### Compound 44, N²-(4-methoxyphenyl)-N⁴-(2-(4-methylpiperazin-1-yl)ethyl)quinazoline-2,4-diamine (23%)

¹H NMR (400 MHz, DMSO-d₆): δ 8.80 (s, 1H), 7.98 (dd, J=8.0 Hz, 0.8 Hz, 1H), 7.94 (t, J=4.8 Hz, 1H), 7.78 (d, J=8.8 Hz, 2H), 7.56-7.52 (m, 1H), 7.34 (dd, J=8.4 Hz, 0.8 Hz, 1H), 7.13-7.09 (m, 1H), 6.84 (d, J=8.8 Hz, 2H), 3.72 (s, 3H), 3.65 (q, J=6.4 Hz, 2H), 2.59 (t, J=7.2 Hz, 2H), 2.50-2.33 (m, 8H), 2.16 (s, 3H). LCMS: m/z 393.41 [M+H]⁺.

### Compound 55, N²-(4-ethylphenyl)-N⁴-(2-(4-methylpiperazin-1-yl)ethyl)quinazoline-2,4-diamine (14%)

¹H NMR (400 MHz, DMSO-d₆): δ 8.89 (s, 1H), 8.01-7.95 (m, 2H), 7.78 (d, J=8.4 Hz, 2H), 7.58-7.54 (m, 1H), 7.36 (dd, J=8.4 Hz, 0.8 Hz, 1H), 7.14-7.16 (m, 1H), 7.07 (d, J=8.8 Hz, 2H), 3.66 (q, J=6.4 Hz, 2H), 2.61-2.53 (m, 4H), 2.50-2.28 (m, 8H), 2.15 (s, 3H), 1.17 (t, J=7.6 Hz, 3H). LCMS: m/z 391.38 [M+H]⁺.

### Compound 56, N²-(3-fluorophenyl)-N⁴-(2-(4-methylpiperazin-1-yl)ethyl)quinazoline-2,4-diamine (16%)

¹H NMR (400 MHz, DMSO-d₆): δ 9.26 (s, 1H), 8.09-8.00 (m, 3H), 7.62-7.56 (m, 2H), 7.42 (d, J=8.0 Hz, 1H), 7.27-7.18 (m, 2H), 6.69-6.65 (m, 1H), 3.69 (q, J=6.4 Hz, 2H), 2.61 (t, J=7.2 Hz, 2H), 2.50-2.26 (m, 8H), 2.14 (s, 3H). LCMS: m/z 381.30 [M+H]⁺.

### Compound 60, N⁴-(2-(dimethylamino)ethyl)-N²-(3-(trifluoromethyl)phenyl)quinazoline-2,4-diamine (13%)

¹H NMR (400 MHz, DMSO-d₆): δ 9.42 (s, 1H), 8.52 (s, 1H), 8.10-8.06 (m, 3H), 7.61 (t, J=7.6 Hz, 1H), 7.46 (t, J=8.0 Hz, 1H), 7.41 (d, J=8.4 Hz, 1H), 7.23-7.18 (m, 2H), 3.67 (q, J=6.4 Hz, 2H), 2.56 (t, J=6.4 Hz, 2H), 2.21 (s, 6H). LCMS: m/z 376.32 [M+H]⁺.

### Compound 61, N⁴-(2-(dimethylamino)ethyl)-N²-(4-methoxyphenyl)quinazoline-2,4-diamine (24%)

¹H NMR (400 MHz, DMSO-d₆): δ 8.80 (s, 1H), 8.00 (d, J=8.0 Hz, 1H), 7.91 (t, J=5.2 Hz, 1H), 7.79 (d, J=9.2 Hz, 2H), 7.54 (t, J=8.0 Hz, 1H), 7.34 (d, J=8.0 Hz, 1H), 7.12 (t, J=8.0 Hz, 1H), 6.84 (d, J=9.2 Hz, 2H), 3.72 (s, 3H), 3.64 (q, J=6.4 Hz, 2H), 2.55-2.51 (m, 2H), 2.22 (s, 6H). LCMS: m/z 338.32 [M+H]⁺.

### Compound 79, N⁴-(2-(1H-imidazol-4-yl)ethyl)-N²-(3-(trifluoromethyl)phenyl)quinazoline-2,4-diamine (11%)

¹H NMR (400 MHz, DMSO-d₆): δ 11.80 (br s, 1H), 9.44 (s, 1H), 8.52 (s, 1H), 8.30 (br s, 1H), 8.14-8.07 (m, 2H), 7.64-7.60 (m, 2H), 7.52-7.41 (m, 2H), 7.23-7.18 (m, 2H), 6.91 (br s, 1H), 3.80 (br s, 2H), 2.98-2.90 (m, 2H). LCMS: m/z 399.30 [M+H]⁺.

### Compound 80, N⁴-(2-(1H-imidazol-4-yl)ethyl)-N²-(4-methoxyphenyl)quinazoline-2,4-diamine (11%)

¹H NMR (400 MHz, DMSO-d₆): δ 11.80 (br s, 1H), 8.83 (s, 1H), 8.16 (br s, 1H), 8.00 (d, J=7.6 Hz, 1H), 7.81 (d, J=8.8 Hz, 2H), 7.59-7.53 (m, 2H), 7.34 (d, J=8.4 Hz, 1H), 7.12 (t, J=7.6 Hz, 1H), 6.91 (br s, 1H), 6.82 (d, J=8.8 Hz, 2H), 3.79-3.71 (m, 5H), 2.96-2.86 (m, 2H). LCMS: m/z 361.34 [M+H]⁺.

### Compound 81, N⁴-(2-(1H-imidazol-4-yl)ethyl)-N²-(4-ethylphenyl)quinazoline-2,4-diamine (17%)

¹H NMR (400 MHz, DMSO-d₆): δ 11.80 (br s, 1H), 8.90 (s, 1H), 8.18 (br s, 1H), 8.01 (d, J=7.6 Hz, 1H), 7.82 (d, J=8.0 Hz, 2H), 7.58-7.53 (m, 2H), 7.37 (d, J=8.4 Hz, 1H), 7.14 (t, J=8.0 Hz, 1H), 7.06 (d, J=8.4 Hz, 2H), 6.92 (br s, 1H), 3.78 (br s, 2H), 2.97-2.87 (m, 2H), 2.55-2.50 (m, 2H), 1.16 (t, J=7.6 Hz, 3H). LCMS: m/z 359.38 [M+H]⁺.

### Compound 82, N⁴-(2-(1H-imidazol-4-yl)ethyl)-N²-(3-fluorophenyl)quinazoline-2,4-diamine (15%)

¹H NMR (400 MHz, DMSO-d₆): δ 11.80 (brs, 1H), 9.28 (s, 1H), 8.28 (brs, 1H), 8.07-8.02 (m, 2H), 7.64-7.52 (m, 3H), 7.43 (d, J=8.0 Hz, 1H), 7.27-7.18 (m, 2H), 6.92 (br s, 1H), 6.66 (td, J=8.4 Hz, 2.0 Hz, 1H), 3.80 (br s, 2H), 2.98-2.88 (m, 2H). LCMS: m/z 349.31 [M+H]⁺.

### Compound 83, N⁴-(2-(1H-imidazol-4-yl)ethyl)-N²-(3-fluorophenyl)quinazoline-2,4-diamine (16%)

¹H NMR (400 MHz, DMSO-d₆): δ 11.80 (br s, 1H), 9.07 (s, 1H), 8.23-8.19 (m, 1H), 8.02 (d, J=8.0 Hz, 1H), 7.96-7.93 (m, 2H), 7.59-7.53 (m, 2H), 7.38 (d, J=8.4 Hz, 1H), 7.16 (t, J=7.6 Hz, 1H), 7.06 (t, J=8.8 Hz, 2H), 6.93 (br s, 1H), 3.81-3.76 (m, 2H), 2.97-2.87 (m, 2H). LCMS: m/z 349.32 [M+H]⁺.

### Compound 99, N¹-(2-(1H-pyrrol-1-yl)quinazolin-4-yl)-N²,N²-dimethylethane-1,2-diamine (16%)

¹H NMR (400 MHz, DMSO-d₆): δ 8.52 (t, J=5.6 Hz, 1H), 8.20 (d, J=8.0 Hz, 1H), 7.79 (t, J=2.4 Hz, 2H), 7.73 (t, J=8.0 Hz, 1H), 7.62 (d, J=7.6 Hz, 1H), 7.41 (t, J=8.0 Hz, 1H), 6.27 (t, J=2.4 Hz, 1H), 3.72 (q, J=6.4 Hz, 2H), 2.58 (t, J=6.8 Hz, 2H), 2.24 (s, 6H). LCMS: m/z 282.42 [M+H]⁺.

### Compound 103, N¹-(2-(1H-pyrrol-1-yl)quinazolin-4-yl)-N³,N³-dimethylpropane-1,3-diamine (17%)

¹H NMR (400 MHz, DMSO-d₆): δ 8.64 (t, J=5.2 Hz, 1H), 8.18 (d, J=8.0 Hz, 1H), 7.80 (t, J=2.4 Hz, 2H), 7.73 (t, J=7.6 Hz, 1H), 7.62 (d, J=8.0 Hz, 1H), 7.40 (t, J=8.0 Hz, 1H), 6.26 (t, J=2.4 Hz, 1H), 3.64 (q, J=6.4 Hz, 2H), 2.35 (t, J=7.2 Hz, 2H), 2.17 (s, 6H), 1.83 (quin, J=6.8 Hz, 2H). LCMS: m/z 296.44 [M+H]⁺.

### Compound 108, N-(2-(1H-imidazol-4-yl)ethyl)-2-(1H-pyrrol-1-yl)quinazolin-4-amine (31%)

¹H NMR (400 MHz, DMSO-d₆): δ 11.80 (br s, 1H), 8.19 (d, J=7.6 Hz, 1H), 7.82 (t, J=2.4 Hz, 2H), 7.75-7.71 (m, 1H), 7.62 (dd, J=8.4 Hz, 0.8 Hz, 1H), 7.57 (d, J=0.8 Hz, 1H), 7.43-7.38 (m, 1H), 6.87 (br s, 1H), 6.27 (t, J=2.4 Hz, 1H), 3.84 (q, J=7.2 Hz, 2H), 2.35 (t, J=7.2 Hz, 2H). LCMS: m/z 305.38 [M+H]⁺.

### Compound 111, 3-((2-(1H-pyrrol-1-yl)quinazolin-4-yl)amino)propan-1-ol (12%)

¹H NMR (400 MHz, DMSO-d₆): δ 8.54 (t, J=5.2 Hz, 1H), 8.21 (dd, J=8.4 Hz, 0.8 Hz, 1H), 7.80 (t, J=2.4 Hz, 2H), 7.75-7.70 (m, 1H), 7.62 (dd, J=8.4 Hz, 0.8 Hz, 1H), 7.42-7.38 (m, 1H), 6.26 (t, J=2.4 Hz, 1H), 4.55 (t, J=4.8 Hz, 1H), 3.64 (q, J=6.4 Hz, 2H), 3.56 (q, J=6.4 Hz, 2H), 1.87 (quin, J=6.4 Hz, 2H). LCMS: m/z 269.35 [M+H]⁺.

### Preparation of Compound 8, N-(3-methyl-2-(4-methylpiperazin-1-yl)butyl)-2-(pyrrolidin-1-yl)quinazolin-4-amine

To a stirred solution of 2-chloro-N-(3-methyl-2-(4-methylpiperazin-1-yl)butyl)quinazolin-4-amine (200 mg, 0.57 mmol) in isopropyl alcohol (4 mL) was added pyrrolidine (122 mg, 1.71 mmol) at room temperature under an atmosphere of nitrogen. The resultant reaction mixture was heated for 30 mins at 100° C. in a microwave reactor. The progress of the reaction was monitored by TLC. After complete consumption of the starting material, the reaction mixture was diluted with H₂O (10 mL) and extracted with ethyl acetate (2×30 mL). The combined organic layer was dried over Na₂SO₄ and concentrated under reduced pressure to obtain the crude product. The crude compound was purified by reverse phase preparative HPLC obtain the title compound as an off-white solid (94 mg, 43%).

¹H NMR (400 MHz, DMSO-d₆): δ 7.93 (dd, J=8.0, 0.8 Hz, 1H), 7.61 (t, J=5.2 Hz, 1H), 7.47-7.43 (m, 1H), 7.24 (dd, J=8.4, 0.8 Hz, 1H), 7.02-6.98 (m, 1H), 3.68-3.64 (m, 1H), 3.57-3.52 (m, 5H), 2.73-2.66 (m, 3H), 2.61-2.56 (m, 2H), 2.20 (br s, 4H), 2.08 (s, 3H), 1.91-1.89 (m, 4H), 1.89-1.71 (m, 1H), 0.98 (d, J=6.4 Hz, 3H), 0.95 (d, J=6.8 Hz, 3H). LCMS: m/z 383.42 [M+H]⁺.

Other analogues prepared by this method:

### Compound 16, N-(2-(4-methylpiperazin-1-yl)ethyl)-2-(pyrrolidin-1-yl)quinazolin-4-amine (19%)

¹H NMR (400 MHz, DMSO-d₆): δ 7.89 (dd, J=8.0 Hz, 0.8 Hz, 1H), 7.80 (t, J=5.2 Hz, 1H), 7.47-7.43 (m, 1H), 7.24 (dd, J=8.4 Hz, 0.8 Hz, 1H), 7.01-6.97 (m, 1H), 3.59 (q, J=6.4 Hz, 2H), 3.52 (br s, 4H), 2.57 (t, J=7.2 Hz, 2H), 2.50-2.31 (m, 8H), 2.14 (s, 3H), 1.91-1.88 (m, 4H). LCMS: m/z 341.37 [M+H]⁺.

### Compound 45, N²-benzyl-N⁴-(2-(4-methylpiperazin-1-yl)ethyl)quinazoline-2,4-diamine (trifluoroacetate salt) (44%)

¹H NMR (400 MHz, DMSO-d₆): δ 12.66 (br s, 1H), 9.49 (br s, 1H), 8.77 (br s, 1H), 8.18 (d, J=8.0 Hz, 1H), 7.82 (t, J=7.6 Hz, 1H), 7.50-6.96 (m, 7H), 4.70 (d, J=6.0 Hz, 2H), 3.68 (br s, 2H), 3.33 (br s, 2H), 2.93 (br s, 4H), 2.75 (s, 3H), 2.65-2.63 (m, 2H), 2.49-2.32 (m, 2H). LCMS: m/z 377.28 [M+H]⁺.

### Compound 46, N⁴-(2-(4-methylpiperazin-1-yl)ethyl)-N²-(3-(trifluoromethyl)benzyl)quinazoline-2,4-diamine (trifluoroacetate salt) (25%)

¹H NMR (400 MHz, DMSO-d₆): δ 12.83 (br s, 1H), 9.51 (br s, 1H), 8.88 (br s, 1H), 8.19 (d, J=8.0 Hz, 1H), 7.88-7.58 (m, 6H), 7.49-7.44 (m, 2H), 4.79 (d, J=6.4 Hz, 2H), 3.76-3.49 (m, 2H), 3.40-3.32 (m, 2H), 2.92 (br s, 4H), 2.75 (s, 3H), 2.62-2.53 (m, 2H), 2.42-2.23 (m, 2H). LCMS: m/z 445.43 [M+H]⁺.

### Compound 47, N⁴-(2-(4-methylpiperazin-1-yl)ethyl)-N²-(4-(trifluoromethyl)benzyl)quinazoline-2,4-diamine (trifluoroacetate salt) (21%)

¹H NMR (400 MHz, DMSO-d₆): δ 12.88 (br s, 1H), 9.52 (br s, 1H), 8.90 (br s, 1H), 8.19 (d, J=8.0 Hz, 1H), 7.84-7.80 (m, 1H), 7.73 (d, J=8.0 Hz, 2H), 7.59 (d, J=8.0 Hz, 2H), 7.50 (d, J=7.6 Hz, 1H), 7.46-7.42 (m, 1H), 4.79 (d, J=5.6 Hz, 2H), 3.62-3.53 (m, 2H), 3.37-3.28 (m, 4H), 2.96-2.81 (m, 4H), 2.75 (s, 3H), 2.40-2.29 (m, 2H). LCMS: m/z 445.43 [M+H]⁺.

### Compound 48, N²-(3-methoxybenzyl)-N⁴-(2-(4-methylpiperazin-1-yl)ethyl)quinazoline-2,4-diamine (29%)

¹H NMR (400 MHz, DMSO-d₆): δ 9.25 (br s, 1H), 8.51 (br s, 1H), 8.12 (br s, 1H), 7.73 (br s, 1H), 7.42-7.23 (m, 3H), 6.93-6.91 (m, 2H), 6.83 (d, J=7.6 Hz, 1H), 4.62 (br s, 2H), 3.73 (s, 3H), 3.64 (br s, 2H), 3.17-2.67 (m, 11H), 2.28-2.20 (s, 2H). LCMS: m/z 407.51 [M+H]⁺.

### Compound 49, N²-(4-methoxybenzyl)-N⁴-(2-(4-methylpiperazin-1-yl)ethyl)quinazoline-2,4-diamine (19%)

¹H NMR (400 MHz, DMSO-d₆): δ 7.89 (d, J=8.4 Hz, 1H), 7.75 (br s, 1H), 7.45 (t, J=7.2 Hz, 1H), 7.26 (d, J=8.0 Hz, 2H), 7.20 (d, J=8.0 Hz, 1H), 7.00 (t, J=7.6 Hz, 1H), 6.98 (br s, 1H), 6.84 (d, J=8.8 Hz, 2H), 4.46 (d, J=6.0 Hz, 2H), 3.70 (s, 3H), 3.55 (q, J=6.0 Hz, 2H), 2.42-2.20 (m, 10H), 2.13 (s, 3H). LCMS: m/z 407.37 [M+H]⁺.

### Compound 50, N⁴-(2-(4-methylpiperazin-1-yl)ethyl)-N²-phenethylquinazoline-2,4-diamine (10%)

¹H NMR (400 MHz, DMSO-d₆): δ 7.90 (dd, J=8.4, 0.8 Hz, 1H), 7.75 (br s, 1H), 7.48-7.43 (m, 1H), 7.29-7.18 (m, 6H), 7.01 (t, J=7.6 Hz, 1H), 6.53 (br s, 1H), 3.60 (br s, 2H), 3.54-3.48 (m, 2H), 2.86 (t, J=7.6 Hz, 2H), 2.56 (t, J=7.2 Hz, 2H), 2.44-2.32 (m, 8H), 2.13 (s, 3H). LCMS: m/z 391.41 [M+H]⁺.

### Compound 51, N⁴-(2-(4-methylpiperazin-1-yl)ethyl)-N²-(3-(trifluoromethyl)phenethyl)quinazoline-2,4-diamine (trifluoroacetate salt) (44%)

¹H NMR (400 MHz, DMSO-d₆): δ 12.67 (br s, 1H), 9.49 (br s, 1H), 8.43 (br s, 1H), 8.18 (d, J=8.0 Hz, 1H), 7.80 (t, J=7.6 Hz, 1H), 7.68-7.53 (m, 4H), 7.43-7.40 (m, 2H), 3.75-3.74 (m, 4H), 3.36-2.67 (m, 15H). LCMS: m/z 457.36 [M−H]⁻.

### Compound 52, N⁴-(2-(4-methylpiperazin-1-yl)ethyl)-N²-(4-(trifluoromethyl)phenethyl)quinazoline-2,4-diamine (trifluoroacetate salt) (68%)

¹H NMR (400 MHz, DMSO-d₆): δ 12.79 (br s, 1H), 9.54 (br s, 1H), 8.52 (br s, 1H), 8.18 (d, J=8.0 Hz, 1H), 7.81 (t, J=7.6 Hz, 1H), 7.68 (d, J=8.0 Hz, 2H), 7.52-7.42 (m, 4H), 3.79-3.74 (m, 4H), 3.23-3.02 (m, 10H), 2.79 (2, 3H), 2.67-2.50 (m, 5H). LCMS: m/z 459.46 [M+H]⁺.

### Compound 53, N²-(3-methoxyphenethyl)-N⁴-(2-(4-methylpiperazin-1-yl)ethyl)quinazoline-2,4-diamine (24%)

¹H NMR (400 MHz, DMSO-d₆): δ 7.90 (d, J=7.2 Hz, 1H), 7.74 (br s, 1H), 7.48-7.43 (m, 1H), 7.28-7.18 (m, 2H), 7.01 (t, J=7.6 Hz, 1H), 6.85-6.82 (m, 2H), 6.76 (dd, J=7.6 Hz, 2.0 Hz, 1H), 6.51 (br s, 1H), 3.73 (s, 3H), 3.62-3.55 (m, 2H), 3.52 (q, J=6.0 Hz, 2H), 2.83 (t, J=7.2 Hz, 2H), 2.56 (t, J=7.2 Hz, 2H), 2.50-2.28 (m, 8H), 2.13 (s, 3H). LCMS: m/z 421.44 [M+H]⁺.

### Compound 54, N²-(4-methoxyphenethyl)-N⁴-(2-(4-methylpiperazin-1-yl)ethyl)quinazoline-2,4-diamine (22%)

¹H NMR (400 MHz, DMSO-d₆): δ 7.89 (d, J=7.6 Hz, 1H), 7.78 (br s, 1H), 7.56-7.44 (m, 1H), 7.28-7.17 (m, 3H), 7.01 (t, J=7.6 Hz, 1H), 6.86 (d, J=8.4 Hz, 2H), 6.59 (br s, 1H), 3.72 (s, 3H), 3.60 (br s, 2H), 3.48 (q, J=6.0 Hz, 2H), 2.79 (t, J=7.2 Hz, 2H), 2.56 (t, J=7.2 Hz, 2H), 2.52-2.32 (m, 8H), 2.15 (s, 3H). LCMS: m/z 421.44 [M+H]1.

### Compound 57, N²-(4-ethylphenethyl)-N⁴-(2-(4-methylpiperazin-1-yl)ethyl)quinazoline-2,4-diamine (11%)

¹H NMR (400 MHz, DMSO-d₆): δ 7.90 (d, J=8.4 Hz, 1H), 7.75 (br s, 1H), 7.46 (t, J=7.6 Hz, 1H), 7.22-7.16 (m, 3H), 7.13 (d, J=8.0 Hz, 2H), 7.00 (t, J=7.6 Hz, 1H), 6.54 (br s, 1H), 3.60 (br s, 2H), 3.49 (q, J=6.4 Hz, 2H), 2.82 (t, J=7.6 Hz, 2H), 2.59-2.53 (m, 4H), 2.50-2.29 (m, 8H), 2.13 (s, 3H), 1.16 (t, J=7.6 Hz, 3H). LCMS: m/z 419.44 [M+H]⁺.

### Compound 58, N²-(3-fluorophenethyl)-N⁴-(2-(4-methylpiperazin-1-yl)ethyl)quinazoline-2,4-diamine (18%)

¹H NMR (400 MHz, DMSO-d₆): δ 7.90 (d, J=7.2 Hz, 1H), 7.77 (br s, 1H), 7.48-7.44 (m, 1H), 7.35-7.30 (m, 1H), 7.23 (br s, 1H), 7.12-7.08 (m, 2H), 7.04-6.99 (m, 2H), 6.56 (br s, 1H), 3.59-3.51 (m, 4H), 2.89 (t, J=7.6 Hz, 2H), 2.57-2.51 (m, 2H), 2.50-2.29 (m, 8H), 2.13 (s, 3H). LCMS: m/z 409.40 [M+H]⁺.

### Compound 59, N²-(4-fluorophenethyl)-N⁴-(2-(4-methylpiperazin-1-yl)ethyl)quinazoline-2,4-diamine (10%)

¹H NMR (400 MHz, DMSO-d₆): δ 7.90 (d, J=7.6 Hz, 1H), 7.79 (br s, 1H), 7.48-7.44 (m, 1H), 7.30 (dd, J=8.4 Hz, 6.0 Hz, 2H), 7.23 (s, 1H), 7.10 (t, J=8.8 Hz, 2H), 7.01 (t, J=7.2 Hz, 1H), 6.54 (br s, 1H), 3.59 (br s, 2H), 3.50 (q, J=6.4 Hz, 2H), 2.86 (t, J=7.6 Hz, 2H), 2.57-2.51 (m, 2H), 2.50-2.44 (m, 8H), 2.13 (s, 3H). LCMS: m/z 409.39 [M+H]⁺.

### Compound 67, N⁴-(2-(dimethylamino)ethyl)-N²-(3-fluorophenethyl)quinazoline-2,4-diamine (17%)

¹H NMR (400 MHz, DMSO-d₆): δ 7.92 (d, J=7.2 Hz, 1H), 7.75 (br s, 1H), 7.48-7.44 (m, 1H), 7.32 (q, J=7.2 Hz, 1H), 7.23 (br s, 1H), 7.11-7.07 (m, 2H), 7.03-6.98 (m, 2H), 6.53 (br s, 1H), 3.58-3.51 (m, 4H), 2.89 (t, J=7.6 Hz, 2H), 2.50-2.48 (m, 2H), 2.19 (s, 6H). LCMS: m/z 354.34 [M+H]⁺.

### Compound 68, N⁴-(2-(dimethylamino)ethyl)-N²-(4-fluorophenethyl)quinazoline-2,4-diamine (19%)

¹H NMR (400 MHz, DMSO-d₆): δ 7.92 (d, J=8.0 Hz, 1H), 7.73 (br s, 1H), 7.46 (t, J=7.6 Hz, 1H), 7.30 (dd, J=8.4 Hz, 6.0 Hz, 2H), 7.23 (br s, 1H), 7.10 (t, J=8.8 Hz, 2H), 7.00 (t, J=7.2 Hz, 1H), 6.51 (br s, 1H), 3.62-3.53 (m, 2H), 3.51 (q, J=6.0 Hz, 2H), 2.86 (t, J=7.6 Hz, 2H), 2.50-2.48 (m, 2H), 2.19 (s, 6H). LCMS: m/z 354.35 [M+H]⁺.

### Compound 75, N⁴-(3-(dimethylamino)propyl)-N²-(4-methoxyphenethyl)quinazoline-2,4-diamine (9%)

¹H NMR (400 MHz, DMSO-d₆): δ 7.90 (dd, J=8.0 Hz, 0.8 Hz, 1H), 7.84 (brs, 1H), 7.48-7.43 (m, 1H), 7.23-7.20 (m, 1H), 7.17 (d, J=8.4 Hz, 2H), 7.00 (t, J=7.6 Hz, 1H), 6.86 (d, J=8.8 Hz, 2H), 6.42 (br s, 1H), 3.72 (s, 3H), 3.50-3.45 (m, 4H), 2.79 (t, J=7.6 Hz, 2H), 2.31 (t, J=6.8 Hz, 2H), 2.15 (s, 6H), 1.79 (quin, J=6.8 Hz, 2H). LCMS: m/z 380.37 [M+H]⁺.

### Compound 76, N⁴-(3-(dimethylamino)propyl)-N²-(4-ethylphenethyl)quinazoline-2,4-diamine (20%)

¹H NMR (400 MHz, DMSO-d₆): δ 7.90 (d, J=7.6 Hz, 1H), 7.84 (br s, 1H), 7.46 (t, J=7.2 Hz, 1H), 7.21 (br s, 1H), 7.17 (d, J=8.0 Hz, 2H), 7.13 (d, J=8.4 Hz, 2H), 7.00 (t, J=7.2 Hz, 1H), 6.46 (br s, 1H), 3.50 (q, J=6.4 Hz, 4H), 2.82 (t, J=7.6 Hz, 2H), 2.55 (q, J=7.6 Hz, 2H), 2.29 (t, J=7.2 Hz, 2H), 2.13 (s, 6H), 1.79 (quin, J=6.8 Hz, 2H), 1.16 (t, J=7.6 Hz, 3H). LCMS: m/z 378.40 [M+H]⁺.

### Compound 77, N⁴-(3-(dimethylamino)propyl)-N²-(3-fluorophenethyl)quinazoline-2,4-diamine (35%)

¹H NMR (400 MHz, DMSO-d₆): δ 7.90 (d, J=7.6 Hz, 1H), 7.88 (br s, 1H), 7.47-7.43 (m, 1H), 7.35-7.30 (m, 1H), 7.22 (br s, 1H), 7.11-7.08 (m, 2H), 7.03-6.98 (m, 2H), 6.52 (br s, 1H), 3.55-3.50 (m, 4H), 2.89 (t, J=7.2 Hz, 2H), 2.29 (t, J=6.8 Hz, 2H), 2.13 (s, 6H), 1.76 (quin, J=7.2 Hz, 2H). LCMS: m/z 368.38 [M+H]⁺.

### Compound 89, 2-((2-((4-ethylphenethyl)amino)quinazolin-4-yl)amino)ethan-1-ol (9%)

¹H NMR (400 MHz, DMSO-d₆): δ 7.96 (d, J=7.2 Hz, 1H), 7.80 (br s, 1H), 7.46 (t, J=8.0 Hz, 1H), 7.23 (br s, 1H), 7.17 (d, J=8.0 Hz, 2H), 7.13 (d, J=8.0 Hz, 2H), 7.00 (t, J=7.6 Hz, 1H), 6.47 (br s, 1H), 4.76 (br s, 1H), 3.63 (br s, 2H), 3.56 (br s, 2H), 3.49 (q, J=6.0 Hz, 2H), 2.82 (t, J=7.6 Hz, 2H), 2.55 (q, J=7.6 Hz, 2H), 1.16 (t, J=7.6 Hz, 3H). LCMS: m/z 337.40 [M+H]⁺.

### Compound 90, 2-((2-((3-fluorophenethyl)amino)quinazolin-4-yl)amino)ethan-1-ol (17%)

¹H NMR (400 MHz, DMSO-d₆): δ 7.96 (d, J=7.2 Hz, 1H), 7.80 (br s, 1H), 7.46 (t, J=7.2 Hz, 1H), 7.36-7.30 (m, 1H), 7.24 (br s, 1H), 7.12-7.08 (m, 2H), 7.04-7.00 (m, 2H), 6.53 (br s, 1H), 4.75 (t, J=5.2 Hz, 1H), 3.64-3.50 (m, 6H), 2.89 (t, J=7.2 Hz, 2H). LCMS: m/z 327.31 [M+H]⁺.

### Compound 91, 2-((2-((4-fluorophenethyl)amino)quinazolin-4-yl)amino)ethan-1-ol (15%)

¹H NMR (400 MHz, DMSO-d₆): δ 7.95 (dd, J=8.0 Hz, 0.8 Hz, 1H), 7.80 (brs, 1H), 7.48-7.44 (m, 1H), 7.29 (dd, J=8.8 Hz, 6.0 Hz, 2H), 7.23 (br d, J=6.4 Hz, 1H), 7.11 (t, J=8.8 Hz, 2H), 7.01 (t, J=7.2 Hz, 1H), 6.51 (br s, 1H), 4.75 (br s, 1H), 3.63 (br s, 2H), 3.56-3.47 (m, 4H), 2.85 (t, J=7.6 Hz, 2H). LCMS: m/z 327.29 [M+H]⁺.

### Compound 92, 3-((2-((3-(trifluoromethyl)phenethyl)amino)quinazolin-4-yl)amino)propan-1-ol (13%)

¹H NMR (400 MHz, DMSO-d₆): δ 7.94 (d, J=8.0 Hz, 1H), 7.79 (br s, 1H), 7.62-7.54 (m, 4H), 7.48-7.44 (m, 1H), 7.22 (br d, J=5.6 Hz, 1H), 7.00 (t, J=7.2 Hz, 1H), 6.57 (br s, 1H), 4.50 (t, J=5.2 Hz, 1H), 3.58-3.48 (m, 6H), 2.97 (t, J=7.2 Hz, 2H), 1.81-1.77 (m, 2H). LCMS: m/z 391.34 [M+H]⁺.

### Compound 93, 3-((2-((4-ethylphenethyl)amino)quinazolin-4-yl)amino)propan-1-ol (9%)

¹H NMR (400 MHz, DMSO-d₆): δ 7.93 (d, J=7.6 Hz, 1H), 7.77 (br s, 1H), 7.46 (t, J=7.2 Hz, 1H), 7.23 (br s, 1H), 7.17 (d, J=8.0 Hz, 2H), 7.13 (d, J=8.0 Hz, 2H), 7.00 (t, J=7.6 Hz, 1H), 6.47 (br s, 1H), 4.50 (t, J=5.2 Hz, 1H), 3.53-3.46 (m, 6H), 2.82 (t, J=7.6 Hz, 2H), 2.56 (q, J=7.6 Hz, 2H), 1.81-1.77 (m, 2H), 1.16 (t, J=7.6 Hz, 3H). LCMS: m/z 351.41 [M+H]⁺.

### Compound 94, 3-((2-((3-fluorophenethyl)amino)quinazolin-4-yl)amino)propan-1-ol (14%)

¹H NMR (400 MHz, DMSO-d₆): δ 7.94 (d, J=7.6 Hz, 1H), 7.78 (br s, 1H), 7.48-7.44 (m, 1H), 7.36-7.30 (m, 1H), 7.23 (br s, 1H), 7.12-7.08 (m, 2H), 7.03-6.99 (m, 2H), 6.53 (br s, 1H), 4.50 (br s, 1H), 3.56-3.49 (m, 6H), 2.89 (t, J=7.6 Hz, 2H), 1.79 (quin, J=6.4 Hz, 2H). LCMS: m/z 341.37 [M+H]⁺.

### Compound 95, 3-((2-((4-fluorophenethyl)amino)quinazolin-4-yl)amino)propan-1-ol (9%)

¹H NMR (400 MHz, DMSO-d₆): δ 7.93 (d, J=7.6 Hz, 1H), 7.78 (br s, 1H), 7.48-7.44 (m, 1H), 7.30 (dd, J=8.4 Hz, 5.6 Hz, 2H), 7.23 (br s, 1H), 7.10 (t, J=9.2 Hz, 2H), 7.00 (t, J=7.2 Hz, 1H), 6.51 (br s, 1H), 4.51 (br s, 1H), 3.53-3.47 (m, 6H), 2.85 (t, J=7.6 Hz, 2H), 1.79 (quin, J=6.4 Hz, 2H). LCMS: m/z 341.37 [M+H]⁺. tert-butyl 4-(2-((2-((3-(trifluoromethyl)phenethyl)amino)quinazolin-4-yl)amino)ethyl)piperazine-1-carboxylate (96%). LCMS: m/z 545.44 [M+H]⁺

### Compound 113, N⁴-(2-morpholinoethyl)-N²-(3-(trifluoromethyl)phenethyl)quinazoline-2,4-diamine (33%)

¹H NMR (400 MHz, DMSO-d₆): δ 7.92 (d, J=8.0 Hz, 1H), 7.79 (br s, 1H), 7.62-7.45 (m, 5H), 7.23 (br s, 1H), 7.01 (t, J=7.6 Hz, 1H), 6.57 (br s, 1H), 3.59-3.56 (m, 8H), 2.98 (t, J=7.2 Hz, 2H), 2.56 (t, J=7.2 Hz, 2H), 2.42 (br s, 4H). LCMS: m/z 446.14 [M+H]⁺.

### Compound 114, N⁴-(2-(piperidin-1-yl)ethyl)-N²-(3-(trifluoromethyl)phenethyl)quinazoline-2,4-diamine (15%)

¹H NMR (400 MHz, DMSO-d₆): δ 7.90 (d, J=8.0 Hz, 1H), 7.70 (br s, 1H), 7.61-7.44 (m, 5H), 7.23 (br s, 1H), 7.01 (t, J=7.2 Hz, 1H), 6.58 (br s, 1H), 3.59-3.55 (m, 4H), 2.98 (t, J=7.2 Hz, 2H), 2.53-2.50 (m, 2H), 2.38-2.32 (m, 4H), 1.46-1.35 (m, 6H). LCMS: m/z 444.32 [M+H]⁺.

### Compound 115, N⁴-(2-(pyrrolidin-1-yl)ethyl)-N²-(3-(trifluoromethyl)phenethyl)quinazoline-2,4-diamine (11%)

¹H NMR (400 MHz, DMSO-d₆): δ 7.92 (d, J=8.0 Hz, 1H), 7.81 (br s, 1H), 7.61-7.44 (m, 5H), 7.23 (br s, 1H), 7.01 (t, J=7.2 Hz, 1H), 6.58 (br s, 1H), 3.59-3.53 (m, 4H), 2.98 (t, J=7.2 Hz, 2H), 2.66 (t, J=6.8 Hz, 2H), 2.50-2.47 (m, 4H), 1.66 (br s, 4H). LCMS: m/z 430.25 [M+H]⁺.

### Compound 116, N⁴-(2-methoxyethyl)-N²-(3-(trifluoromethyl)phenethyl)quinazoline-2,4-diamine (21%)

¹H NMR (400 MHz, DMSO-d₆): δ 7.95 (d, J=8.0 Hz, 1H), 7.88 (br s, 1H), 7.62-7.45 (m, 5H), 7.24 (br s, 1H), 7.01 (t, J=7.2 Hz, 1H), 6.60 (br s, 1H), 3.64-3.55 (m, 6H), 3.27 (s, 3H), 2.98 (t, J=7.2 Hz, 2H). LCMS: m/z 391.14 [M+H]⁺.

### Compound 117, N⁴-(3-methoxypropyl)-N²-(3-(trifluoromethyl)phenethyl)quinazoline-2,4-diamine (45%)

¹H NMR (400 MHz, DMSO-d₆): δ 7.94 (d, J=8.0 Hz, 1H), 7.81 (br s, 1H), 7.62-7.44 (m, 5H), 7.23 (br s, 1H), 7.01 (t, J=7.2 Hz, 1H), 6.57 (br s, 1H), 3.59-3.50 (m, 4H), 3.40 (t, J=6.0 Hz, 2H), 3.23 (s, 3H), 2.97 (t, J=7.2 Hz, 2H), 1.87 (quin, J=6.8 Hz, 2H). LCMS: m/z 405.15 [M+H]⁺.

tert-butyl (2-((2-((3-(trifluoromethyl)phenethyl)amino)quinazolin-4-yl)amino)ethyl)carbamate (41%). LCMS: m/z 476.48 [M+H]⁺.

tert-butyl (2-((2-((3-(trifluoromethyl)phenethyl)amino)quinazolin-4-yl)amino)propyl)carbamate (37%). LCMS: m/z 490.21 [M+H]⁺.

### Compound 120, N⁴-(2-(4-methylpiperazin-1-yl)ethyl)-N²-(2-(trifluoromethyl)phenethyl)quinazoline-2,4-diamine (17%)

¹H NMR (400 MHz, DMSO-d₆): δ 7.91 (d, J=8.0 Hz, 1H), 7.76 (br s, 1H), 7.69 (d, J=7.6 Hz, 1H), 7.62-7.55 (m, 2H), 7.49-7.40 (m, 2H), 7.23 (br s, 1H), 7.02 (t, J=7.2 Hz, 1H), 6.71 (br s, 1H), 3.61-3.54 (m, 4H), 3.06 (t, J=6.8 Hz, 2H), 2.55 (t, J=7.2 Hz, 2H), 2.50-2.19 (m, 8H), 2.13 (s, 3H). LCMS: m/z 459.34 [M+H]⁺.

### Compound 121, N²-(2-fluorophenethyl)-N⁴-(2-(4-methylpiperazin-1-yl)ethyl)quinazoline-2,4-diamine (9%)

¹H NMR (400 MHz, DMSO-d₆): δ 7.90 (d, J=8.0 Hz, 1H), 7.76 (br s, 1H), 7.46 (t, J=7.2 Hz, 1H), 7.35 (t, J=7.6 Hz, 1H), 7.28-7.11 (m, 4H), 7.01 (t, J=7.6 Hz, 1H), 6.60 (br s, 1H), 3.61-3.49 (m, 4H), 2.91 (t, J=7.2 Hz, 2H), 2.55 (t, J=7.2 Hz, 2H), 2.50-2.21 (m, 8H), 2.13 (s, 3H). LCMS: m/z 409.38 [M+H]⁺.

### Compound 122, N²-(2-methylphenethyl)-N⁴-(2-(4-methylpiperazin-1-yl)ethyl)quinazoline-2,4-diamine (17%)

¹H NMR (400 MHz, DMSO-d₆): δ 7.91 (d, J=7.6 Hz, 1H), 7.73 (br s, 1H), 7.47 (t, J=7.6 Hz, 1H), 7.20-7.08 (m, 5H), 7.01 (t, J=7.6 Hz, 1H), 6.59 (br s, 1H), 3.59 (br s, 2H), 3.45 (br s, 2H), 2.85 (t, J=7.6 Hz, 2H), 2.56 (t, J=7.2 Hz, 2H), 2.36-2.29 (m, 11H), 2.14 (s, 3H). LCMS: m/z 405.58 [M+H]⁺.

### Compound 123, N²-(2-chlorophenethyl)-N⁴-(2-(4-methylpiperazin-1-yl)ethyl)quinazoline-2,4-diamine (17%)

¹H NMR (400 MHz, DMSO-d₆): δ 7.90 (d, J=8.0 Hz, 1H), 7.76 (br s, 1H), 7.48-7.38 (m, 3H), 7.29-7.18 (m, 3H), 7.01 (t, J=7.6 Hz, 1H), 6.61 (br s, 1H), 3.64-3.51 (m, 4H), 3.00 (t, J=7.6 Hz, 2H), 2.55 (t, J=7.2 Hz, 2H), 2.50-2.22 (m, 8H), 2.13 (s, 3H). LCMS: m/z 425.26 [M+H]⁺.

### Compound 124, N²-(3-methylphenethyl)-N⁴-(2-(4-methylpiperazin-1-yl)ethyl)quinazoline-2,4-diamine (23%)

¹H NMR (400 MHz, DMSO-d₆): δ 7.90 (d, J=8.0 Hz, 1H), 7.76 (br s, 1H), 7.46 (t, J=7.6 Hz, 1H), 7.23-7.16 (m, 2H), 7.08-7.00 (m, 4H), 6.51 (br s, 1H), 3.60 (br s, 2H), 3.50 (q, J=6.0 Hz, 2H), 2.82 (t, J=7.6 Hz, 2H), 2.56 (t, J=7.2 Hz, 2H), 2.50-2.25 (m, 11H), 2.13 (s, 3H). LCMS: m/z 405.46 [M+H]⁺.

### Compound 125, N²-(3-chlorophenethyl)-N⁴-(2-(4-methylpiperazin-1-yl)ethyl)quinazoline-2,4-diamine (47%)

¹H NMR (400 MHz, DMSO-d₆): δ 7.90 (d, J=8.0 Hz, 1H), 7.76 (br s, 1H), 7.46 (t, J=7.6 Hz, 1H), 7.34-7.30 (m, 2H), 7.26-7.20 (m, 3H), 7.01 (t, J=7.6 Hz, 1H), 6.56 (br s, 1H), 3.64-3.50 (m, 4H), 2.88 (t, J=7.2 Hz, 2H), 2.55 (t, J=7.2 Hz, 2H), 2.50-2.25 (m, 8H), 2.13 (s, 3H). LCMS: m/z 425.21 [M+H]⁺.

### Compound 126, 3-(2-((4-((2-(4-methylpiperazin-1-yl)ethyl)amino)quinazolin-2-yl)amino)ethyl)benzonitrile (11%)

¹H NMR (400 MHz, DMSO-d₆): δ 7.90 (d, J=7.6 Hz, 1H), 7.76 (br s, 1H), 7.73 (s, 1H), 7.66 (d, J=7.6 Hz, 1H), 7.62 (d, J=8.0 Hz, 1H), 7.51-7.44 (m, 2H), 7.23 (br s, 1H), 7.01 (t, J=7.2 Hz, 1H), 6.59 (br s, 1H), 3.64-3.54 (m, 4H), 2.94 (t, J=7.2 Hz, 2H), 2.55 (t, J=7.2 Hz, 2H), 2.50-2.23 (m, 8H), 2.13 (s, 3H). LCMS: m/z 416.36 [M+H]⁺.

### Compound 127, N²-(4-methylphenethyl)-N⁴-(2-(4-methylpiperazin-1-yl)ethyl)quinazoline-2,4-diamine (29%)

¹H NMR (400 MHz, DMSO-d₆): δ 7.90 (d, J=8.0 Hz, 1H), 7.76 (br s, 1H), 7.46 (t, J=7.6 Hz, 1H), 7.22 (br s, 1H), 7.15 (d, J=8.0 Hz, 2H), 7.10 (d, J=7.6 Hz, 2H), 7.00 (t, J=7.6 Hz, 1H), 6.51 (br s, 1H), 3.59 (br s, 2H), 3.50 (q, J=6.0 Hz, 2H), 2.81 (t, J=7.6 Hz, 2H), 2.55 (t, J=7.2 Hz, 2H), 2.49-2.24 (m, 11H), 2.14 (s, 3H). LCMS: m/z 405.46 [M+H]⁺.

### Compound 128, N²-(4-isopropylphenethyl)-N⁴-(2-(4-methylpiperazin-1-yl)ethyl)quinazoline-2,4-diamine (23%)

¹H NMR (400 MHz, DMSO-d₆): δ 7.90 (d, J=8.0 Hz, 1H), 7.78 (br s, 1H), 7.46 (t, J=8.0 Hz, 1H), 7.19-7.15 (m, 5H), 7.00 (t, J=7.2 Hz, 1H), 6.51 (br s, 1H), 3.60 (br s, 2H), 3.49 (q, J=6.4 Hz, 2H), 2.88-2.80 (m, 3H), 2.56 (t, J=7.2 Hz, 2H), 2.44-2.27 (m, 8H), 2.13 (s, 3H), 1.19 (d, J=6.8 Hz, 6H). LCMS: m/z 433.43 [M+H]⁺.

### Compound 129, N²-(4-(tert-butyl)phenethyl)-N⁴-(2-(4-methylpiperazin-1-yl)ethyl)quinazoline-2,4-diamine (15%)

¹H NMR (400 MHz, DMSO-d₆): δ 7.90 (d, J=8.0 Hz, 1H), 7.75 (br s, 1H), 7.46 (t, J=7.6 Hz, 1H), 7.31 (d, J=8.0 Hz, 2H), 7.23-7.17 (m, 3H), 7.00 (t, J=7.6 Hz, 1H), 6.52 (br s, 1H), 3.60 (br s, 2H), 3.50 (q, J=6.4 Hz, 2H), 2.82 (t, J=7.6 Hz, 2H), 2.56 (t, J=6.8 Hz, 2H), 2.45-2.29 (m, 8H), 2.13 (s, 3H), 1.27 (s, 9H). LCMS: m/z 447.65 [M+H]⁺.

### Compound 130, N²-(4-chlorophenethyl)-N⁴-(2-(4-methylpiperazin-1-yl)ethyl)quinazoline-2,4-diamine (32%)

¹H NMR (400 MHz, DMSO-d₆): δ 7.90 (d, J=8.0 Hz, 1H), 7.78 (br s, 1H), 7.46 (t, J=8.0 Hz, 1H), 7.34 (d, J=8.4 Hz, 2H), 7.29 (d, J=8.4 Hz, 2H), 7.23 (br s, 1H), 7.01 (t, J=7.6 Hz, 1H), 6.54 (br s, 1H), 3.59-3.48 (m, 4H), 2.86 (t, J=7.2 Hz, 2H), 2.55 (t, J=7.2 Hz, 2H), 2.44-2.28 (m, 8H), 2.14 (s, 3H). LCMS: m/z 425.37 [M+H]⁺.

### Compound 131, 4-(2-((4-((2-(4-methylpiperazin-1-yl)ethyl)amino)quinazolin-2-yl)amino)ethyl)benzonitrile (14%)

¹H NMR (400 MHz, DMSO-d₆): δ 7.90 (d, J=7.6 Hz, 1H), 7.78-7.74 (m, 3H), 7.48-7.44 (m, 3H), 7.23 (br s, 1H), 7.01 (t, J=7.6 Hz, 1H), 6.60 (br s, 1H), 3.61-3.52 (m, 4H), 2.97 (t, J=7.2 Hz, 2H), 2.54 (t, J=7.2 Hz, 2H), 2.49-2.22 (m, 8H), 2.14 (s, 3H). LCMS: m/z 414.30 [M−H]⁻.

### Compound 132, N²-(4-hydroxyphenethyl)-N⁴-(2-(4-methylpiperazin-1-yl)ethyl)quinazoline-2,4-diamine (23%)

¹H NMR (400 MHz, DMSO-d₆): δ 9.14 (s, 1H), 7.91 (d, J=8.0 Hz, 1H), 7.75 (br s, 1H), 7.45 (t, J=7.6 Hz, 1H), 7.22 (br s, 1H), 7.06-6.98 (m, 3H), 6.68 (d, J=8.0 Hz, 2H), 6.47 (br s, 1H), 3.60 (br s, 2H), 3.45 (q, J=6.4 Hz, 2H), 2.73 (t, J=7.6 Hz, 2H), 2.56 (t, J=6.8 Hz, 2H), 2.45-2.30 (m, 8H), 2.14 (s, 3H). LCMS: m/z 407.39 [M+H]⁺.

### Compound 133, N²-(4-aminophenethyl)-N⁴-(2-(4-methylpiperazin-1-yl)ethyl)quinazoline-2,4-diamine (27%)

¹H NMR (400 MHz, DMSO-d₆): δ 7.89 (d, J=8.0 Hz, 1H), 7.75 (br s, 1H), 7.45 (t, J=7.6 Hz, 1H), 7.21 (br s, 1H), 7.00 (t, J=7.6 Hz, 1H), 6.90 (d, J=8.4 Hz, 2H), 6.50 (d, J=8.0 Hz, 2H), 6.39 (br s, 1H), 4.84 (s, 2H), 3.60 (br s, 2H), 3.41 (q, J=6.4 Hz, 2H), 2.66 (t, J=7.6 Hz, 2H), 2.56 (t, J=7.2 Hz, 2H), 2.45-2.24 (m, 8H), 2.14 (s, 3H). LCMS: m/z 406.31 [M+H]⁺.

### Compound 134, N²-(4-(dimethylamino)phenethyl)-N⁴-(2-(4-methylpiperazin-1-yl)ethyl)quinazoline-2,4-diamine (12%)

¹H NMR (400 MHz, DMSO-d₆): δ 7.90 (d, J=8.0 Hz, 1H), 7.76 (br s, 1H), 7.45 (t, J=7.2 Hz, 1H), 7.22 (br s, 1H), 7.08 (d, J=8.8 Hz, 2H), 7.00 (t, J=7.2 Hz, 1H), 6.67 (d, J=8.8 Hz, 2H), 6.47 (br s, 1H), 3.60 (br s, 2H), 3.45 (q, J=6.4 Hz, 2H), 2.85 (s, 6H), 2.73 (t, J=7.6 Hz, 2H), 2.56 (t, J=7.2 Hz, 2H), 2.49-2.22 (m, 8H), 2.13 (s, 3H). LCMS: m/z 434.37 [M+H]⁺.

### Compound 135, N²-(3,4-dichlorophenethyl)-N⁴-(2-(4-methylpiperazin-1-yl)ethyl)quinazoline-2,4-diamine (9%)

¹H NMR (400 MHz, DMSO-d₆): δ 7.90 (d, J=8.0 Hz, 1H), 7.74 (br s, 1H), 7.53-7.50 (m, 2H), 7.46 (t, J=7.6 Hz, 1H), 7.27-7.22 (m, 2H), 7.01 (t, J=7.2 Hz, 1H), 6.57 (br s, 1H), 3.59-3.50 (m, 4H), 2.88 (t, J=7.2 Hz, 2H), 2.54 (t, J=7.2 Hz, 2H), 2.48-2.20 (m, 8H), 2.13 (s, 3H). LCMS: m/z 459.19 [M+H]⁺.

### Compound 136, N²-(3,4-difluorophenethyl)-N⁴-(2-(4-methylpiperazin-1-yl)ethyl)quinazoline-2,4-diamine (12%)

¹H NMR (400 MHz, DMSO-d₆): δ 7.90 (d, J=8.0 Hz, 1H), 7.76 (br s, 1H), 7.46 (t, J=7.6 Hz, 1H), 7.35-7.27 (m, 2H), 7.23 (br s, 1H), 7.10 (br s, 1H), 7.01 (t, J=7.6 Hz, 1H), 6.56 (br s, 1H), 3.59-3.49 (m, 4H), 2.87 (t, J=7.2 Hz, 2H), 2.54 (t, J=7.2 Hz, 2H), 2.48-2.22 (m, 8H), 2.13 (s, 3H). LCMS: m/z 427.36 [M+H]⁺.

### Compound 137, N²-(3,4-dimethoxyphenethyl)-N⁴-(2-(4-methylpiperazin-1-yl)ethyl)quinazoline-2,4-diamine (30%)

¹H NMR (400 MHz, DMSO-d₆): δ 7.90 (d, J=8.0 Hz, 1H), 7.78 (br s, 1H), 7.46 (t, J=8.0 Hz, 1H), 7.22 (br s, 1H), 7.00 (t, J=7.2 Hz, 1H), 6.87-6.85 (m, 2H), 6.77 (d, J=8.0 Hz, 1H), 6.54 (br s, 1H), 3.74 (s, 3H), 3.71 (s, 3H), 3.59 (br s, 2H), 3.52 (q, J=6.4 Hz, 2H), 2.79 (t, J=7.6 Hz, 2H), 2.55 (t, J=6.8 Hz, 2H), 2.44-2.22 (m, 8H), 2.13 (s, 3H). LCMS: m/z 451.45 [M+H]⁺.

### Compound 138, N²-(3,5-difluorophenethyl)-N⁴-(2-(4-methylpiperazin-1-yl)ethyl)quinazoline-2,4-diamine (13%)

¹H NMR (400 MHz, DMSO-d₆): δ 7.90 (d, J=7.2 Hz, 1H), 7.78 (br s, 1H), 7.46 (t, J=7.2 Hz, 1H), 7.23 (br s, 1H), 7.06-6.99 (m, 4H), 6.58 (br s, 1H), 3.62-3.51 (m, 4H), 2.91 (t, J=6.8 Hz, 2H), 2.55 (t, J=6.8 Hz, 2H), 2.44-2.19 (m, 8H), 2.13 (s, 3H). LCMS: m/z 427.36 [M+H]⁺.

### Compound 139, N²-(3,5-dichlorophenethyl)-N⁴-(2-(4-methylpiperazin-1-yl)ethyl)quinazoline-2,4-diamine (12%)

¹H NMR (400 MHz, DMSO-d₆): δ 7.90 (d, J=8.0 Hz, 1H), 7.76 (br s, 1H), 7.46 (t, J=7.6 Hz, 1H), 7.41 (s, 1H), 7.34 (s, 2H), 7.23 (br s, 1H), 7.01 (t, J=7.2 Hz, 1H), 6.58 (br s, 1H), 3.62-3.53 (m, 4H), 2.89 (t, J=6.8 Hz, 2H), 2.55 (t, J=6.8 Hz, 2H), 2.50-2.22 (m, 8H), 2.13 (s, 3H). LCMS: m/z 459.19 [M+H]⁺.

### Compound 140, N²-(3,5-dimethoxyphenethyl)-N⁴-(2-(4-methylpiperazin-1-yl)ethyl)quinazoline-2,4-diamine (10%)

¹H NMR (400 MHz, DMSO-d₆): δ 7.90 (d, J=8.0 Hz, 1H), 7.75 (br s, 1H), 7.46 (t, J=7.6 Hz, 1H), 7.22 (br s, 1H), 7.01 (t, J=7.2 Hz, 1H), 6.87-6.85 (m, 2H), 6.77 (d, J=8.0 Hz, 1H), 6.54 (br s, 1H), 6.42 (s, 2H), 6.33 (s, 1H), 3.72 (s, 6H), 3.59 (br s, 2H), 3.51 (q, J=6.4 Hz, 2H), 2.79 (t, J=7.2 Hz, 2H), 2.55 (t, J=6.8 Hz, 2H), 2.44-2.22 (m, 8H), 2.13 (s, 3H). LCMS: m/z 451.49 [M+H]⁺.

### Compound 141, N⁴-(2-(4-methylpiperazin-1-yl)ethyl)-N²-(3,4,5-trifluorophenethyl)quinazoline-2,4-diamine (8%)

¹H NMR (400 MHz, DMSO-d₆): δ 7.90 (d, J=7.2 Hz, 1H), 7.78 (br s, 1H), 7.46 (t, J=7.2 Hz, 1H), 7.24-7.20 (m, 3H), 7.01 (t, J=7.6 Hz, 1H), 6.58 (br s, 1H), 3.62-3.50 (m, 4H), 2.87 (t, J=6.8 Hz, 2H), 2.54 (t, J=7.2 Hz, 2H), 2.50-2.22 (m, 8H), 2.13 (s, 3H). LCMS: m/z 445.2 [M+H]⁺.

### Compound 142, N⁴-(2-(4-methylpiperazin-1-yl)ethyl)-N²-(3,4,5-trimethoxyphenethyl)quinazoline-2,4-diamine (3%)

¹H NMR (400 MHz, DMSO-d₆): δ 7.90 (d, J=8.0 Hz, 1H), 7.74 (br s, 1H), 7.46 (t, J=7.2 Hz, 1H), 7.23 (br s, 1H), 7.00 (t, J=7.6 Hz, 1H), 6.55 (br s, 3H), 3.75 (s, 6H), 3.61 (s, 3H), 3.60-3.51 (m, 4H), 2.80 (t, J=7.2 Hz, 2H), 2.55 (t, J=7.2 Hz, 2H), 2.50-2.22 (m, 8H), 2.12 (s, 3H). LCMS: m/z 481.33 [M+H]⁺.

### Compound 143, N²-(2-(benzo[d][1,3]dioxol-5-yl)ethyl)-N⁴-(2-(4-methylpiperazin-1-yl)ethyl)quinazoline-2,4-diamine (6%)

¹H NMR (400 MHz, DMSO-d₆): δ 7.90 (d, J=8.0 Hz, 1H), 7.75 (br s, 1H), 7.46 (t, J=7.6 Hz, 1H), 7.22 (br s, 1H), 7.00 (t, J=7.6 Hz, 1H), 6.84-6.80 (m, 2H), 6.71 (J=8.0 Hz, 1H), 6.47 (br s, 1H), 5.96 (s, 2H), 3.59 (br s, 2H), 3.47 (q, J=6.0 Hz, 2H), 2.78 (t, J=7.6 Hz, 2H), 2.55 (t, J=7.2 Hz, 2H), 2.50-2.23 (m, 8H), 2.13 (s, 3H). LCMS: m/z 435.29 [M+H]⁺.

### Compound 144, N⁴-(2-(4-methylpiperazin-1-yl)ethyl)-N²-(2-(pyridin-2-yl)ethyl)quinazoline-2,4-diamine (35%)

¹H NMR (400 MHz, DMSO-d₆): δ 8.50 (d, J=4.0 Hz, 1H), 7.90 (d, J=8.0 Hz, 1H), 7.72-7.68 (m, 2H), 7.46 (t, J=7.2 Hz, 1H), 7.30 (d, J=7.6 Hz, 1H), 7.23-7.20 (m, 2H), 7.01 (t, J=7.6 Hz, 1H), 6.53 (br s, 1H), 3.68-3.57 (m, 4H), 3.02 (t, J=7.6 Hz, 2H), 2.55 (t, J=7.2 Hz, 2H), 2.44-2.28 (m, 8H), 2.13 (s, 3H). LCMS: m/z 392.39 [M+H]⁺.

### Compound 145, N⁴-(2-(4-methylpiperazin-1-yl)ethyl)-N²-(2-(pyridin-3-yl)ethyl)quinazoline-2,4-diamine (16%)

¹H NMR (400 MHz, DMSO-d₆): δ 8.47 (d, J=1.6 Hz, 1H), 8.40 (dd, J=4.4 Hz, 1.2 Hz, 1H), 7.90 (d, J=8.0 Hz, 1H), 7.77 (br s, 1H), 7.68 (d, J=7.6 Hz, 1H), 7.46 (t, J=8.0 Hz, 1H), 7.31 (dd, J=7.6 Hz, 4.8 Hz, 1H), 7.23 (br s, 1H), 7.01 (t, J=7.2 Hz, 1H), 6.60 (br s, 1H), 3.64-3.51 (m, 4H), 2.89 (t, J=6.8 Hz, 2H), 2.55 (t, J=7.2 Hz, 2H), 2.49-2.24 (m, 8H), 2.13 (s, 3H). LCMS: m/z 392.42 [M+H]⁺.

### Compound 146, N⁴-(2-(4-methylpiperazin-1-yl)ethyl)-N²-(2-(pyridin-4-yl)ethyl)quinazoline-2,4-diamine (26%)

¹H NMR (400 MHz, DMSO-d₆): δ 8.46 (d, J=5.6 Hz, 2H), 7.90 (d, J=8.0 Hz, 1H), 7.76 (brs, 1H), 7.46 (t, J=8.0 Hz, 1H), 7.29 (d, J=5.6 Hz, 2H), 7.24 (br s, 1H), 7.01 (t, J=7.2 Hz, 1H), 6.60 (br s, 1H), 3.62-3.53 (m, 4H), 2.90 (t, J=7.2 Hz, 2H), 2.55 (t, J=6.8 Hz, 2H), 2.49-2.24 (m, 8H), 2.13 (s, 3H). LCMS: m/z 392.39 [M+H]⁺.

### Compound 147, N⁴-(2-(4-methylpiperazin-1-yl)ethyl)-N²-(2-(thiophen-2-yl)ethyl)quinazoline-2,4-diamine (24%)

¹H NMR (400 MHz, DMSO-d₆): δ 7.90 (d, J=8.0 Hz, 1H), 7.78 (br s, 1H), 7.46 (t, J=7.2 Hz, 1H), 7.33 (dd, J=4.8 Hz, 0.8 Hz, 1H), 7.23 (br s, 1H), 7.01 (t, J=7.6 Hz, 1H), 6.96-6.91 (m, 2H), 6.61 (br s, 1H), 3.62-3.52 (m, 4H), 3.08 (t, J=7.6 Hz, 2H), 2.56 (t, J=7.2 Hz, 2H), 2.49-2.23 (m, 8H), 2.13 (s, 3H). LCMS: m/z 397.23 [M+H]⁺.

### Compound 148, N²-(2-(furan-2-yl)ethyl)-N⁴-(2-(4-methylpiperazin-1-yl)ethyl)quinazoline-2,4-diamine (13%)

¹H NMR (400 MHz, DMSO-d₆): δ 7.90 (d, J=8.0 Hz, 1H), 7.77 (br s, 1H), 7.51 (d, J=1.2 Hz, 1H), 7.46 (t, J=8.0 Hz, 1H), 7.23 (br s, 1H), 7.01 (t, J=7.6 Hz, 1H), 6.57 (br s, 1H), 6.36 (dd, J=4.8 Hz, 2.0 Hz, 1H), 6.17 (d, J=2.8 Hz, 1H), 3.61-3.53 (m, 4H), 2.90 (t, J=7.2 Hz, 2H), 2.55 (t, J=7.2 Hz, 2H), 2.49-2.22 (m, 8H), 2.14 (s, 3H). LCMS: m/z 381.34 [M+H]⁺.

### Compound 152, N²-(2-(1-methyl-1H-pyrrol-2-yl)ethyl)-N⁴-(2-(4-methylpiperazin-1-yl)ethyl)quinazoline-2,4-diamine (18%)

¹H NMR (400 MHz, DMSO-d₆): δ 7.90 (d, J=8.0 Hz, 1H), 7.73 (br s, 1H), 7.46 (t, J=8.0 Hz, 1H), 7.22 (br d, J=6.8 Hz, 1H), 7.01 (t, J=7.6 Hz, 1H), 6.60 (br s, 1H), 6.55 (br s, 1H), 5.87 (t, J=6.8 Hz, 1H), 5.82 (br s, 1H), 3.57 (br s, 5H), 3.48 (q, J=6.4 Hz, 2H), 2.79 (t, J=7.6 Hz, 2H), 2.55 (t, J=7.2 Hz, 2H), 2.49-2.23 (m, 8H), 2.14 (s, 3H). LCMS: m/z 394.52 [M+H]⁺.

### Preparation of Compound 9, N-(3-methyl-2-(4-methylpiperazin-1-yl)butyl)-2-(1H-pyrrol-1-yl)quinazolin-4-amine

To a stirred solution of 2-chloro-N-(3-methyl-2-(4-methylpiperazin-1-yl)butyl)quinazolin-4-amine (100 mg, 0.29 mmol) and 1H-pyrrole (30 mg, 0.44 mmol) in 1,4-dioxane (5 mL) was added Ru-Phos (28 mg, 0.06 mmol) followed by Cs₂CO₃ (283 mg, 0.87 mmol) under a nitrogen atmosphere at room temperature. The reaction mixture was degassed with nitrogen for 10 min, after which was added Pd₂(dba)₃ (28 mg, 0.03 mmol) at room temperature. The reaction mixture was stirred at 110° C. for 16 h. The progress of the reaction was monitored by TLC. After complete consumption of the starting material, the reaction mixture was quenched with H₂O (30 mL) and extracted with EtOAc (2×50 mL). The combined organic layer was dried over anhydrous Na₂SO₄ and concentrated under reduced pressure to obtain the crude product. The crude compound was purified by reverse phase preparative HPLC to obtain the title compound as an off-white solid (44 mg, 41%).

¹H NMR (400 MHz, DMSO-d₆): δ 8.35 (t, J=5.2 Hz, 1H), 8.22 (d, J=8.0 Hz, 1H), 7.80 (t, J=2.4 Hz, 2H), 7.73 (t, J=7.2 Hz, 1H), 7.62 (d, J=8.0 Hz, 1H), 7.42 (t, J=8.0 Hz, 1H), 6.27 (t, J=2.0 Hz, 2H), 3.75 (t, J=6.0 Hz, 2H), 2.76-2.59 (m, 5H), 2.18 (br s, 4H), 2.06 (s, 3H), 1.83-1.78 (m, 1H), 1.04 (d, J=6.8 Hz, 3H), 0.98 (d, J=6.8 Hz, 3H). LCMS: m/z 379.34 [M+H]⁺.

Other analogues prepared by this method:

### Compound 17, N-(2-(4-methylpiperazin-1-yl)ethyl)-2-(1H-pyrrol-1-yl)quinazolin-4-amine (25%)

¹H NMR (400 MHz, DMSO-d₆): δ 8.52 (t, J=5.6 Hz, 1H), 8.19 (d, J=8.0 Hz, 1H), 7.79 (t, J=2.4 Hz, 2H), 7.76-7.71 (m, 1H), 7.62 (dd, J=8.4 Hz, 0.8 Hz, 1H), 7.43-7.39 (m, 1H), 6.27 (t, J=2.4 Hz, 2H), 3.73 (q, J=6.8 Hz, 2H), 2.64 (t, J=6.8 Hz, 2H), 2.50-2.32 (m, 8H), 2.14 (s, 3H). LCMS: m/z 337.34 [M+H]⁺.

### Compound 40, N⁴-(2-(4-methylpiperazin-1-yl)ethyl)-N²-phenylquinazoline-2,4-diamine (24%)

¹H NMR (400 MHz, DMSO-d₆): δ 8.99 (s, 1H), 8.03-7.99 (m, 2H), 7.90 (d, J=7.6 Hz, 2H), 7.59-7.55 (m, 1H), 7.39 (dd, J=8.4 Hz, 0.8 Hz, 1H), 7.25-7.21 (m, 2H), 7.17-7.13 (m, 1H), 6.88 (t, J=7.2 Hz, 1H), 3.69 (q, J=6.8 Hz, 2H), 2.60 (t, J=7.2 Hz, 2H), 2.49-2.32 (m, 8H), 2.15 (s, 3H). LCMS: m/z 363.35 [M+H]⁺.

### Compound 41, N⁴-(2-(4-methylpiperazin-1-yl)ethyl)-N²-(3-(trifluoromethyl)phenyl)quinazoline-2,4-diamine (44%)

¹H NMR (400 MHz, DMSO-d₆): δ 9.42 (s, 1H), 8.50 (s, 1H), 8.10-8.06 (m, 3H), 7.63-7.59 (m, 1H), 7.46 (t, J=8.0 Hz, 1H), 7.41 (dd, J=8.4 Hz, 0.8 Hz, 1H), 7.23-7.21 (m, 2H), 3.69 (q, J=6.4 Hz, 2H), 2.61 (t, J=6.8 Hz, 2H), 2.33-2.30 (m, 8H), 2.14 (s, 3H). LCMS: m/z 431.44 [M+H]⁺.

### Compound 42, N⁴-(2-(4-methylpiperazin-1-yl)ethyl)-N²-(4-(trifluoromethyl)phenyl)quinazoline-2,4-diamine (37%)

¹H NMR (400 MHz, DMSO-d₆): δ 9.49 (s, 1H), 8.14-8.12 (m, 3H), 8.06 (dd, J=8.0 Hz, 0.8 Hz, 1H), 7.64-7.56 (m, 3H), 7.45 (dd, J=8.0 Hz, 0.8 Hz, 1H), 7.24-7.23 (m, 1H), 3.69 (q, J=6.4 Hz, 2H), 2.61 (t, J=7.2 Hz, 2H), 2.33-2.32 (m, 8H), 2.15 (s, 3H). LCMS: m/z 431.31 [M+H]⁺.

### Compound 43, N²-(3-methoxyphenyl)-N⁴-(2-(4-methylpiperazin-1-yl)ethyl)quinazoline-2,4-diamine (37%)

¹H NMR (400 MHz, DMSO-d₆): δ 8.98 (s, 1H), 8.04-7.98 (m, 2H), 7.73 (br s, 1H), 7.66-7.60 (m, 1H), 7.42-7.37 (m, 2H), 7.20-7.15 (m, 2H), 6.52 (dd, J=8.0 Hz, 2.0 Hz, 1H), 3.75 (s, 3H), 3.69 (q, J=6.4 Hz, 2H), 2.61 (t, J=7.2 Hz, 2H), 2.50-2.34 (m, 8H), 2.15 (s, 3H). LCMS: m/z 398.38 [M+H]⁺.

### Compound 62, N⁴-(2-(dimethylamino)ethyl)-N²-(4-ethylphenyl)quinazoline-2,4-diamine (39%)

¹H NMR (400 MHz, DMSO-d₆): δ 8.88 (s, 1H), 8.02 (d, J=7.6 Hz, 1H), 7.95 (t, J=5.2 Hz, 1H), 7.79 (d, J=8.4 Hz, 2H), 7.57-7.53 (m, 1H), 7.36 (d, J=8.0 Hz, 1H), 7.15-7.11 (m, 1H), 7.07 (d, J=8.4 Hz, 2H), 3.65 (q, J=6.4 Hz, 2H), 2.56-2.50 (m, 4H), 2.22 (s, 6H), 1.16 (t, J=7.6 Hz, 3H). LCMS: m/z 336.33 [M+H]⁺.

### Compound 63, N⁴-(2-(dimethylamino)ethyl)-N²-(3-fluorophenyl)quinazoline-2,4-diamine (38%)

¹H NMR (400 MHz, DMSO-d₆): δ 9.27 (s, 1H), 8.07-8.01 (m, 3H), 7.62-7.57 (m, 2H), 7.42 (d, J=8.4 Hz, 1H), 7.27-7.17 (m, 2H), 6.67 (td, J=8.4 Hz, 2.0 Hz, 1H), 3.67 (q, J=6.4 Hz, 2H), 2.56 (t, J=6.8 Hz, 2H), 2.22 (s, 6H). LCMS: m/z 326.26 [M+H]⁺.

### Compound 64, N⁴-(2-(dimethylamino)ethyl)-N²-(3-(trifluoromethyl)phenethyl)quinazoline-2,4-diamine (25%)

¹H NMR (400 MHz, DMSO-d₆): δ 7.91 (d, J=7.6 Hz, 1H), 7.73 (br s, 1H), 7.62-7.49 (m, 4H), 7.46 (t, J=7.2 Hz, 1H), 7.23 (br s, 1H), 7.01 (t, J=7.2 Hz, 1H), 6.58 (br s, 1H), 3.56 (q, J=6.8 Hz, 4H), 2.98 (t, J=7.6 Hz, 2H), 2.50-2.47 (m, 2H), 2.18 (s, 6H). LCMS: m/z 405.35 [M+H]⁺.

### Compound 65, N⁴-(2-(dimethylamino)ethyl)-N²-(4-methoxyphenethyl)quinazoline-2,4-diamine (36%)

¹H NMR (400 MHz, DMSO-d₆): δ 7.91 (d, J=7.6 Hz, 1H), 7.72 (br s, 1H), 7.46 (t, J=7.2 Hz, 1H), 7.22 (br s, 1H), 7.17 (d, J=8.8 Hz, 2H), 7.00 (t, J=7.6 Hz, 1H), 6.85 (d, J=8.4 Hz, 2H), 6.45 (br s, 1H), 3.72 (s, 3H), 3.57 (br s, 2H), 3.47 (q, J=6.4 Hz, 2H), 2.78 (t, J=7.2 Hz, 2H), 2.50-2.49 (m, 2H), 2.19 (s, 6H). LCMS: m/z 366.38 [M+H]⁺.

### Compound 66, N⁴-(2-(dimethylamino)ethyl)-N²-(4-ethylphenethyl)quinazoline-2,4-diamine (15%)

¹H NMR (400 MHz, DMSO-d₆): δ 7.91 (d, J=8.0 Hz, 1H), 7.73 (br s, 1H), 7.46 (t, J=7.2 Hz, 1H), 7.22 (br s, 1H), 7.17 (d, J=8.0 Hz, 2H), 7.12 (d, J=8.0 Hz, 2H), 7.00 (t, J=7.6 Hz, 1H), 6.49 (br s, 1H), 3.57 (br s, 2H), 3.50 (q, J=6.4 Hz, 2H), 2.82 (t, J=7.6 Hz, 2H), 2.59-2.50 (m, 4H), 2.19 (s, 6H), 1.16 (t, J=7.2 Hz, 1H). LCMS: m/z 364.40 [M+H]⁺.

### Compound 69, N⁴-(3-(dimethylamino)propyl)-N²-(3-(trifluoromethyl)phenyl)quinazoline-2,4-diamine (7%)

¹H NMR (400 MHz, DMSO-d₆): δ 9.41 (s, 1H), 8.59 (s, 1H), 8.24 (t, J=5.2 Hz, 1H), 8.06 (d, J=7.6 Hz, 1H), 8.02 (d, J=8.4 Hz, 1H), 7.61 (t, J=8.0 Hz, 1H), 7.46 (t, J=8.0 Hz, 1H), 7.40 (d, J=8.0 Hz, 1H), 7.23-7.19 (m, 2H), 3.59 (q, J=6.8 Hz, 2H), 2.33 (t, J=7.2 Hz, 2H), 2.15 (s, 6H), 1.82 (quin, J=7.2 Hz, 2H). LCMS: m/z 388.35 [M−H]⁻.

### Compound 70, N⁴-(3-(dimethylamino)propyl)-N²-(4-methoxyphenyl)quinazoline-2,4-diamine (38%)

¹H NMR (400 MHz, DMSO-d₆): δ 8.79 (s, 1H), 8.07 (t, J=5.2 Hz, 1H), 7.99 (d, J=7.6 Hz, 1H), 7.80 (d, J=8.8 Hz, 2H), 7.56-7.51 (m, 1H), 7.34 (d, J=8.0 Hz, 1H), 7.13-7.09 (m, 1H), 6.84 (d, J=8.8 Hz, 2H), 3.72 (s, 3H), 3.55 (q, J=6.4 Hz, 2H), 2.33 (t, J=6.8 Hz, 2H), 2.15 (s, 6H), 1.80 (quin, J=6.8 Hz, 2H). LCMS: m/z 352.34 [M+H]⁺.

### Compound 71, N⁴-(3-(dimethylamino)propyl)-N²-(4-ethylphenyl)quinazoline-2,4-diamine (20%)

¹H NMR (400 MHz, DMSO-d₆): δ 8.88 (s, 1H), 8.11 (t, J=5.2 Hz, 1H), 8.00 (dd, J=8.4 Hz, 0.8 Hz, 1H), 7.80 (d, J=8.8 Hz, 2H), 7.56-7.53 (m, 1H), 7.36 (dd, J=8.4 Hz, 0.8 Hz, 1H), 7.13-7.09 (m, 1H), 7.08 (d, J=8.4 Hz, 2H), 3.56 (q, J=6.4 Hz, 2H), 2.54 (q, J=7.6 Hz, 2H), 2.33 (t, J=7.2 Hz, 2H), 2.16 (s, 6H), 1.81 (quin, J=7.2 Hz, 2H), 1.17 (t, J=7.6 Hz, 3H). LCMS: m/z 350.41 [M+H]⁺.

### Compound 72, N⁴-(3-(dimethylamino)propyl)-N²-(3-fluorophenyl)quinazoline-2,4-diamine (27%)

¹H NMR (400 MHz, DMSO-d₆): δ 9.27 (s, 1H), 8.23 (t, J=5.2 Hz, 1H), 8.08-8.03 (m, 2H), 7.61-7.55 (m, 2H), 7.42 (d, J=7.6 Hz, 1H), 7.27-7.17 (m, 2H), 6.67 (td, J=8.4 Hz, 2.4 Hz, 1H), 3.35 (q, J=6.4 Hz, 2H), 2.34 (t, J=7.2 Hz, 2H), 2.15 (s, 6H), 1.82 (quin, J=7.2 Hz, 2H). LCMS: m/z 340.32 [M+H]⁺.

### Compound 73, N⁴-(3-(dimethylamino)propyl)-N²-(4-fluorophenyl)quinazoline-2,4-diamine (14%)

¹H NMR (400 MHz, DMSO-d₆): δ 9.03 (s, 1H), 8.14 (t, J=5.2 Hz, 1H), 8.01 (d, J=7.6 Hz, 1H), 7.93 (dd, J=8.8 Hz, 5.2 Hz, 2H), 7.58-7.54 (m, 1H), 7.38 (d, J=8.0 Hz, 1H), 7.16 (t, J=7.6 Hz, 1H), 7.08 (t, J=8.8 Hz, 2H), 3.56 (q, J=6.8 Hz, 2H), 2.33 (t, J=7.2 Hz, 2H), 2.16 (s, 6H), 1.80 (quin, J=7.2 Hz, 2H). LCMS: m/z 340.32 [M+H]⁺.

### Preparation of Compound 74, N⁴-(3-(dimethylamino)propyl)-N²-(3-(trifluoromethyl)phenethyl)quinazoline-2,4-diamine

To a stirred solution of N¹-(2-chloroquinazolin-4-yl)-N³,N³-dimethylpropane-1,3-diamine (150 mg, 0.57 mmol) in DMSO (10 mL) was added 2-(3-(trifluoromethyl)phenyl)ethan-1-amine (431 mg, 2.28 mmol) at room temperature under an argon atmosphere. The resultant reaction mixture was heated under microwave conditions at 110° C. for 1 h. The progress of the reaction was monitored by TLC. After complete consumption of the starting material, the reaction mixture was concentrated under reduced pressure to obtain the crude product. The crude compound was purified by reverse phase preparative HPLC to obtain the title compound as an off-white solid (32 mg, 14%).

¹H NMR (400 MHz, DMSO-d₆): δ 7.94-7.83 (m, 2H), 7.61-7.52 (m, 4H), 7.47-7.43 (m, 1H), 7.22 (br d, J=6.8 Hz, 1H), 7.01 (t, J=7.6 Hz, 1H), 6.55 (br s, 1H), 3.56 (q, J=6.4 Hz, 2H), 3.52-3.47 (m, 2H), 2.96 (t, J=7.2 Hz, 2H), 2.28 (t, J=7.2 Hz, 2H), 2.12 (s, 6H), 1.76 (quin, J=7.2 Hz, 2H). LCMS: m/z 418.37 [M+H]⁺.

Other analogues prepared by this method:

### Compound 78, N⁴-(3-(dimethylamino)propyl)-N²-(4-fluorophenethyl)quinazoline-2,4-diamine (17%)

¹H NMR (400 MHz, DMSO-d₆): δ 7.94-7.83 (m, 2H), 7.47-7.43 (m, 1H), 7.29 (dd, J=8.4 Hz, 5.6 Hz, 2H), 7.22 (br d, J=6.6 Hz, 1H), 7.11 (t, J=8.8 Hz, 2H), 7.00 (t, J=7.2 Hz, 1H), 6.49 (br s, 1H), 3.52-3.47 (m, 4H), 2.86 (t, J=7.2 Hz, 2H), 2.29 (t, J=7.2 Hz, 2H), 2.13 (s, 6H), 1.76 (quin, J=7.2 Hz, 2H). LCMS: m/z 368.36 [M+H]⁺.

### Compound 84, N⁴-(2-(1H-imidazol-4-yl)ethyl)-N²-(3-(trifluoromethyl)phenethyl)quinazoline-2,4-diamine (11%)

¹H NMR (400 MHz, DMSO-d₆): δ 11.85 (br s, 1H), 7.96-7.90 (m, 2H), 7.62-7.45 (m, 6H), 7.24 (br s, 1H), 7.01 (t, J=7.2 Hz, 1H), 6.88 (br s, 1H), 6.60 (br s, 1H), 3.69 (br s, 2H), 3.57 (q, J=6.4 Hz, 2H), 3.00-2.83 (m, 4H). LCMS: m/z 427.39 [M+H]⁺.

### Compound 85, N⁴-(2-(1H-imidazol-4-yl)ethyl)-N²-(4-methoxyphenethyl)quinazoline-2,4-diamine (9%)

¹H NMR (400 MHz, DMSO-d₆): δ 11.81 (br s, 1H), 8.04-7.88 (m, 2H), 7.56 (br s, 1H), 7.47 (t, J=7.6 Hz, 1H), 7.23 (br s, 1H), 7.16 (d, J=7.6 Hz, 2H), 7.01 (t, J=7.6 Hz, 1H), 6.89-6.80 (m, 3H), 6.50 (br s, 1H), 3.76-3.66 (m, 5H), 3.48 (q, J=6.4 Hz, 2H), 2.88 (br s, 2H), 2.79 (t, J=7.2 Hz, 2H). LCMS: m/z 389.37 [M+H]⁺.

### Compound 86, N⁴-(2-(1H-imidazol-4-yl)ethyl)-N²-(4-ethylphenethyl)quinazoline-2,4-diamine (27%)

¹H NMR (400 MHz, DMSO-d₆): δ 11.79 (br s, 1H), 7.99-7.88 (m, 2H), 7.57 (br s, 1H), 7.46 (t, J=7.2 Hz, 1H), 7.23-7.03 (m, 5H), 7.01 (t, J=7.6 Hz, 1H), 6.89 (br s, 1H), 6.51 (br s, 1H), 3.71 (br s, 2H), 3.50 (q, J=6.8 Hz, 2H), 2.93-2.80 (m, 4H), 2.56 (q, J=7.6 Hz, 2H), 1.16 (t, J=7.6 Hz, 3H). LCMS: m/z 387.38 [M+H]⁺.

### Compound 87, N⁴-(2-(1H-imidazol-4-yl)ethyl)-N²-(3-fluorophenethyl)quinazoline-2,4-diamine (45%)

¹H NMR (400 MHz, DMSO-d₆): δ 11.78 (br s, 1H), 8.04-7.90 (m, 2H), 7.56 (br s, 1H), 7.46 (t, J=7.2 Hz, 1H), 7.38-7.24 (m, 2H), 7.10 (d, J=8.0 Hz, 2H), 7.08-6.99 (m, 2H), 6.88 (br s, 1H), 6.57 (br s, 1H), 3.70 (br s, 2H), 3.54 (q, J=6.8 Hz, 2H), 2.91-2.84 (m, 4H). LCMS: m/z 377.38 [M+H]⁺.

### Compound 88, N⁴-(2-(1H-imidazol-4-yl)ethyl)-N²-(4-fluorophenethyl)quinazoline-2,4-diamine (25%)

¹H NMR (400 MHz, DMSO-d₆): δ 11.79 (br s, 1H), 8.02-7.90 (m, 2H), 7.58 (br s, 1H), 7.46 (t, J=8.0 Hz, 1H), 7.29-7.24 (m, 3H), 7.08 (d, J=8.8 Hz, 2H), 7.01 (t, J=7.6 Hz, 1H), 6.89 (br s, 1H), 6.55 (br s, 1H), 3.71 (br s, 2H), 3.50 (q, J=7.2 Hz, 2H), 2.94-2.83 (m, 4H). LCMS: m/z 377.30 [M+H]⁺.

### Preparation of Compound 112, N⁴-(2-(piperazin-1-yl)ethyl)-N²-(3-(trifluoromethyl)phenethyl)quinazoline-2,4-diamine

To a stirred solution of tert-butyl 4-(2-((2-((3-(trifluoromethyl)phenethyl)amino)quinazolin-4-yl)amino)ethyl)piperazine-1-carboxylate (300 mg, 0.55 mmol) in 1,4-dioxane (1.5 mL) was added 4M HCl in 1,4-dioxane (3 mL) at 0° C. The reaction mixture was stirred at room temperature for 16 h. The progress of the reaction was monitored by TLC. After complete consumption of starting material, the reaction mixture was concentrated under reduced pressure to obtain the crude product. The crude compound was purified by reverse phase preparative HPLC to obtain the title compound as an off-white solid (38 mg, 16%).

¹H NMR (400 MHz, DMSO-d₆): δ 7.91 (d, J=8.0 Hz, 1H), 7.77 (br s, 1H), 7.66-7.44 (m, 5H), 7.23 (br s, 1H), 7.01 (t, J=7.6 Hz, 1H), 6.59 (br s, 1H), 3.62-3.53 (m, 4H), 2.98 (t, J=7.2 Hz, 2H), 2.65 (br s, 4H), 2.54-2.51 (m, 2H), 2.35 (br. s, 4H). LCMS: m/z 445.56 [M+H]⁺.

Other analogues prepared by this method:

### Compound 118, N⁴-(2-aminoethyl)-N²-(3-(trifluoromethyl)phenethyl)quinazoline-2,4-diamine (24%)

¹H NMR (400 MHz, DMSO-d₆): δ 7.97 (d, J=8.0 Hz, 1H), 7.76 (br s, 1H), 7.63-7.44 (m, 5H), 7.23 (br s, 1H), 7.01 (t, J=7.6 Hz, 1H), 6.56 (br s, 1H), 3.56 (q, J=6.4 Hz, 2H), 3.47 (br s, 2H), 2.98 (t, J=7.2 Hz, 2H), 2.79 (t, J=6.4 Hz, 2H). LCMS: m/z 376.21 [M+H]⁺.

### Compound 119, N⁴-(3-aminopropyl)-N²-(3-(trifluoromethyl)phenethyl)quinazoline-2,4-diamine (31%)

¹H NMR (400 MHz, DMSO-d₆): δ 7.95-7.90 (m, 2H), 7.62-7.43 (m, 5H), 7.23 (br s, 1H), 7.00 (t, J=7.6 Hz, 1H), 6.57 (br s, 1H), 3.58-3.52 (m, 4H), 2.98 (t, J=7.2 Hz, 2H), 2.63 (t, J=6.4 Hz, 2H), 1.69 (quin, J=6.4 Hz, 2H). LCMS: m/z 390.31 [M+H]⁺.

### Preparation of Compound 150, N-(2-(4-methylpiperazin-1-yl)ethyl)-2-(3-(trifluoromethyl)phenethoxy)quinazolin-4-amine

To a stirred solution of 2-(3-(trifluoromethyl)phenyl)ethan-1-ol (187 mg, 0.98 mmol) in THF (2 mL), was added NaH (60% dispersion in mineral oil) (52 mg, 1.30 mmol) at 0° C. The mixture was stirred for 30 min. A solution of 2-chloro-N-(2-(4-methylpiperazin-1-yl)ethyl)quinazolin-4-amine (200 mg, 0.65 mmol) in DMF (2 mL) was added drop wise to the reaction mixture. The resultant reaction mixture was stirred at 90° C. for 16 h. The progress of the reaction was monitored by TLC. After complete consumption of the starting material, the reaction mixture was quenched with crushed ice and extracted with EtOAc (2×100 mL). The combined organic layer was dried over anhydrous Na₂SO₄, filtered and concentrated under reduced pressure. The crude compound was purified by reverse phase preparative HPLC to obtain the title compound as a colourless gummy solid (59 mg, 20%).

¹H NMR (400 MHz, DMSO-d₆): δ 8.22 (t, J=5.2 Hz, 1H), 8.10 (d, J=8.4 Hz, 1H), 7.71 (s, 1H), 7.66-7.53 (m, 4H), 7.46 (d, J=8.0 Hz, 1H), 7.29 (t, J=7.6 Hz, 1H), 4.54 (t, J=6.8 Hz, 2H), 3.59 (q, J=6.4 Hz, 2H), 3.16 (t, J=6.8 Hz, 2H), 2.55 (t, J=6.8 Hz, 2H), 2.44-2.28 (m, 8H), 2.13 (s, 3H). LCMS: m/z 460.29 [M+H]⁺.

Other analogues prepared by this method:

### Compound 151, N-(2-(4-methylpiperazin-1-yl)ethyl)-2-((3-(trifluoromethyl)phenethyl)thio)quinazolin-4-amine (13%)

¹H NMR (400 MHz, DMSO-d₆): δ 8.30 (t, J=5.2 Hz, 1H), 8.12 (d, J=8.0 Hz, 1H), 7.72-7.64 (m, 3H), 7.60-7.51 (m, 3H), 7.39 (t, J=8.0 Hz, 1H), 3.62 (q, J=6.4 Hz, 2H), 4.54 (t, J=6.8 Hz, 2H), 3.12 (t, J=6.8 Hz, 2H), 2.55 (t, J=7.2 Hz, 2H), 2.43-2.18 (m, 8H), 2.12 (s, 3H). LCMS: m/z 476.44 [M+H]⁺.

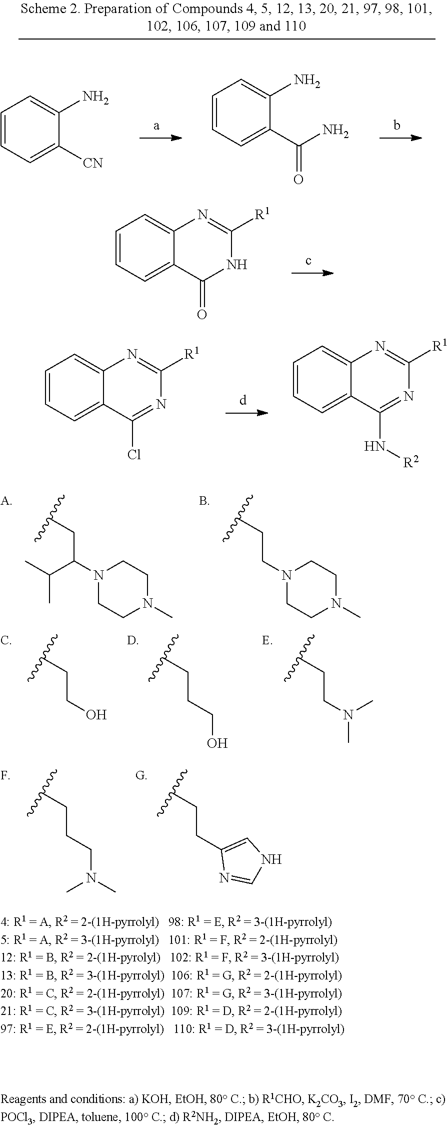

### Preparation of 2-aminobenzamide

To a stirred solution of 2-aminobenzonitrile (5.00 g, 42.32 mmol) in EtOH (20 mL), was added KOH (2.40 g, 42.32 mmol). The resultant reaction mixture was stirred for 16 h at 80° C. The progress of the reaction was monitored by TLC. After complete consumption of the starting material, the reaction mixture was diluted with water and extracted with EtOAc (100 mL). The organic layer was dried over anhydrous Na₂SO₄ and concentrated under reduced pressure to obtain the crude product. This crude compound was purified by column chromatography using 100-200 silica-gel eluting with 30% EtOAc in petroleum ether to obtain the title compound as an off-white solid (3.0 g, 52%).

¹H NMR (400 MHz, DMSO-d₆): δ 7.70 (br s, 1H), 7.51 (dd, J=8.0 Hz, 2.4 Hz, 1H), 7.14-7.09 (m, 1H), 7.04 (br s, 1H), 6.66 (dd, J=8.4 Hz, 2.1 Hz, 1H), 6.48-6.44 (m, 3H).

### Preparation of 2-(1H-pyrrol-2-yl)quinazolin-4(3H)-one

To a stirred solution of 2-aminobenzamide (1.00 g, 7.35 mmol) and 1H-pyrrole-2-carbaldehyde (698 mg, 7.35 mmol) in DMF (20 mL), was added K₂CO₃ (1.00 g, 7.35 mmol) followed by iodine (2.3 g, 8.81 mmol). The resultant reaction mixture was stirred for 16 h at 70° C. The progress of the reaction was monitored by TLC. After complete consumption of the starting material, the reaction mixture was diluted with water and extracted with EtOAc (100 mL). The organic layer was dried over anhydrous Na₂SO₄ and concentrated under reduced pressure to obtain the crude product. This crude compound was purified by column chromatography using 100-200 silica-gel eluting with 20% EtOAc in petroleum ether to obtain the title compound as an off-white solid (230 mg, 15%). LCMS: m/z 212.14 [M+H]⁺.

Other analogues prepared by this method:

2-(1H-pyrrol-3-yl)quinazolin-4(3H)-one (39%). LCMS: m/z 210.37 [M−H]⁻.

### Preparation of 4-chloro-2-(1H-pyrrol-2-yl)quinazoline

To a stirred solution of 2-(1H-pyrrol-2-yl)quinazolin-4(3H)-one (230 mg, 1.09 mmol) in toluene (5 mL), was added POCl₃ (0.5 mL, 5.44 mmol) followed by DIPEA (0.95 mL, 5.44 mmol). The resultant reaction mixture was stirred for 16 h at 100° C. The progress of the reaction was monitored by TLC. After complete consumption of the starting material, the reaction mixture was concentrated under reduced pressure, washed with NaHCO₃ solution and extracted with EtOAc (100 mL). The organic layer was dried over anhydrous Na₂SO₄ and concentrated under reduced pressure to obtain the crude product. This crude compound was purified by column chromatography using 100-200 silica-gel eluting with 25% EtOAc in petroleum ether to obtain the title compound as a brown liquid (180 mg, 72%). LCMS: m/z 230.17 [M+H]⁺.

Other analogues prepared by this method:

4-chloro-2-(1H-pyrrol-3-yl)quinazoline (21%). LCMS: m/z 230.24 [M+H]⁺.

### Preparation of Compound 4, N-(3-methyl-2-(4-methylpiperazin-1-yl)butyl)-2-(1H-pyrrol-2-yl)quinazolin-4-amine

To a stirred solution of 4-chloro-2-(1H-pyrrol-2-yl)quinazoline (120 mg, 0.52 mmol) in ethanol (4 mL) was added DIPEA (0.27 mL, 1.56 mmol) followed by 3-methyl-2-(4-methylpiperazin-1-yl)butan-1-amine (96 mg, 0.52 mmol) at room temperature under an argon atmosphere. The resultant reaction mixture was stirred at room temperature for 16 h. The progress of the reaction was monitored by TLC. After complete consumption of the starting material, the reaction mixture was extracted with EtOAc (2×20 mL) and washed with water (10 mL). The combined organic layer was dried over Na₂SO₄, filtered, and concentrated under reduced pressure to obtain the crude product. The crude compound was purified by reverse phase preparative HPLC to obtain the title compound as an off-white solid (60 mg, 30%).

¹H NMR (400 MHz, DMSO-d₆): δ 11.31 (s, 1H), 8.14 (d, J=7.6 Hz, 1H), 7.85 (t, J=5.2 Hz, 1H), 7.70-7.66 (m, 1H), 7.62 (d, J=7.6 Hz, 1H), 7.39-7.35 (m, 1H), 6.89 (t, J=2.4 Hz, 2H), 6.15 (q, J=2.8 Hz, 1H), 3.83-3.72 (m, 2H), 2.73-2.60 (m, 5H), 2.19 (br s, 4H), 2.06 (s, 3H), 1.84-1.77 (m, 1H), 1.04 (d, J=6.8 Hz, 3H), 0.98 (d, J=6.4 Hz, 3H). LCMS: m/z 379.36 [M+H]⁺.

Other analogues prepared by this method:

### Compound 5, N-(3-methyl-2-(4-methylpiperazin-1-yl)butyl)-2-(1H-pyrrol-3-yl)quinazolin-4-amine (31%)

¹H NMR (400 MHz, DMSO-d₆): δ 11.03 (s, 1H), 8.11 (d, J=8.0 Hz, 1H), 7.78 (t, J=5.2 Hz, 1H), 7.67-7.63 (m, 1H), 7.58 (d, J=7.6 Hz, 1H), 7.51 (br s, 1H), 7.35-7.31 (m, 1H), 6.78 (q, J=2.4 Hz, 1H), 6.73 (d, J=1.2 Hz, 1H), 3.78-3.70 (m, 1H), 3.68-3.65 (m, 1H), 2.77-2.61 (m, 5H), 2.20 (br s, 4H), 2.07 (s, 3H), 1.82-1.77 (m, 1H), 1.05 (d, J=6.8 Hz, 3H), 0.98 (d, J=6.4 Hz, 3H). LCMS: m/z 379.36 [M+H]⁺.

### Compound 12, N-(2-(4-methylpiperazin-1-yl)ethyl)-2-(1H-pyrrol-2-yl)quinazolin-4-amine (45%)

¹H NMR (400 MHz, DMSO-d₆): δ 11.32 (s, 1H), 8.12 (d, J=7.6 Hz, 1H), 8.03 (t, J=5.2 Hz, 1H), 7.70-7.66 (m, 1H), 7.62 (dd, J=8.4 Hz, 0.8 Hz, 1H), 7.38-7.34 (m, 1H), 6.90-6.86 (m, 2H), 6.16-6.14 (m, 1H), 3.77 (q, J=6.4 Hz, 2H), 2.62 (t, J=6.8 Hz, 2H), 2.50-2.31 (m, 8H), 2.14 (s, 3H). LCMS: m/z 337.34 [M+H]⁺.

### Compound 13, N-(2-(4-methylpiperazin-1-yl)ethyl)-2-(1H-pyrrol-3-yl)quinazolin-4-amine (31%)

¹H NMR (400 MHz, DMSO-d₆): δ 11.03 (s, 1H), 8.08 (d, J=8.4 Hz, 1H), 7.96 (t, J=5.2 Hz, 1H), 7.65 (t, J=7.6 Hz, 1H), 7.58 (d, J=8.0 Hz, 1H), 7.50 (br s, 1H), 7.32 (t, J=7.2 Hz, 1H), 6.78 (d, J=2.0 Hz, 1H), 6.72 (br s, 1H), 3.72 (q, J=6.4 Hz, 2H), 2.63 (t, J=7.2 Hz, 2H), 2.50-2.32 (m, 8H), 2.14 (s, 3H). LCMS: m/z 337.32 [M+H]⁺.

### Compound 20, 2-((2-(1H-pyrrol-2-yl)quinazolin-4-yl)amino)ethan-1-ol (18%)

¹H NMR (400 MHz, DMSO-d₆): δ 11.31 (s, 1H), 8.17 (d, J=7.6 Hz, 1H), 8.13 (t, J=5.2 Hz, 1H), 7.70-7.66 (m, 1H), 7.61 (dd, J=8.4 Hz, 0.8 Hz, 1H), 7.37-7.33 (m, 1H), 6.90-6.87 (m, 2H), 6.15 (q, J=2.4 Hz, 1H), 4.80 (t, J=5.6 Hz, 1H), 3.75-3.66 (m, 4H). LCMS: m/z 255.35 [M+H]⁺.

### Compound 21, 2-((2-(1H-pyrrol-3-yl)quinazolin-4-yl)amino)ethan-1-ol (trifluoroacetate salt) (32%)

¹H NMR (400 MHz, DMSO-d₆): δ 13.17 (br s, 1H), 11.88 (s, 1H), 9.84 (br s, 1H), 8.41 (d, J=8.0 Hz, 1H), 8.02-7.95 (m, 2H), 7.85 (d, J=8.0 Hz, 1H), 7.66 (t, J=8.0 Hz, 1H), 7.06 (d, J=2.4 Hz, 1H), 7.01 (br s, 1H), 4.94 (br s, 1H), 3.84 (q, J=5.6 Hz, 2H), 3.74 (d, J=5.5 Hz, 2H). LCMS: m/z 255.24 [M+H]⁺.

### Compound 97, N¹-(2-(1H-pyrrol-2-yl)quinazolin-4-yl)-N²,N²-dimethylethane-1,2-diamine (18%)

¹H NMR (400 MHz, DMSO-d₆): δ 11.33 (s, 1H), 8.13 (d, J=8.0 Hz, 1H), 8.03 (t, J=5.6 Hz, 1H), 7.70-7.66 (m, 1H), 7.61 (d, J=7.6 Hz, 1H), 7.37-7.33 (m, 1H), 6.89-6.87 (m, 2H), 6.15 (q, J=2.4 Hz, 1H), 3.76 (q, J=6.4 Hz, 2H), 2.56 (t, J=6.8 Hz, 2H), 2.24 (s, 6H). LCMS: m/z 282.42 [M+H]⁺.

### Compound 98, N¹-(2-(1H-pyrrol-3-yl)quinazolin-4-yl)-N²,N²-dimethylethane-1,2-diamine (12%)

¹H NMR (400 MHz, DMSO-d₆): δ 11.02 (s, 1H), 8.09 (d, J=7.6 Hz, 1H), 7.95 (t, J=5.2 Hz, 1H), 7.67-7.62 (m, 1H), 7.58 (dd, J=8.0 Hz, 0.8 Hz, 1H), 7.51-7.49 (m, 1H), 7.34-7.29 (m, 1H), 6.78 (q, J=2.4 Hz, 1H), 6.73-6.71 (m, 1H), 3.71 (q, J=6.8 Hz, 2H), 2.56 (t, J=7.2 Hz, 2H), 2.24 (s, 6H). LCMS: m/z 282.31 [M+H]⁺.

### Compound 101, N¹-(2-(1H-pyrrol-2-yl)quinazolin-4-yl)-N³,N³-dimethylpropane-1,3-diamine (12%)

¹H NMR (400 MHz, DMSO-d₆): δ 11.33 (s, 1H), 8.20 (t, J=5.6 Hz, 1H), 8.11 (dd, J=8.4 Hz, 0.8 Hz, 1H), 7.70-7.66 (m, 1H), 7.61 (dd, J=8.4 Hz, 0.8 Hz, 1H), 7.37-7.33 (m, 1H), 6.90-6.86 (m, 2H), 6.16-6.13 (m, 1H), 3.67 (q, J=6.8 Hz, 2H), 2.36 (t, J=7.2 Hz, 2H), 2.17 (s, 6H), 1.82 (quin, J=7.2 Hz, 2H). LCMS: m/z 296.34 [M+H]⁺.

### Compound 102, N¹-(2-(1H-pyrrol-3-yl)quinazolin-4-yl)-N³,N³-dimethylpropane-1,3-diamine (9%)

¹H NMR (400 MHz, DMSO-d₆): δ 11.03 (s, 1H), 8.09-8.06 (m, 2H), 7.66-7.63 (m, 1H), 7.60 (dd, J=8.0 Hz, 1.2 Hz, 1H), 7.51 (d, J=1.2 Hz, 1H), 7.34-7.29 (m, 1H), 6.78 (q, J=2.4 Hz, 1H), 6.73-6.71 (m, 1H), 3.61 (q, J=6.4 Hz, 2H), 2.35 (t, J=6.8 Hz, 2H), 2.17 (s, 6H), 1.83 (quin, J=7.2 Hz, 2H). LCMS: m/z 296.39 [M+H]⁺.

### Compound 106, N-(2-(1H-imidazol-4-yl)ethyl)-2-(1H-pyrrol-2-yl)quinazolin-4-amine (41%)

¹H NMR (400 MHz, DMSO-d₆): δ 11.82 (br s, 1H), 11.33 (s, 1H), 8.31 (br s, 1H), 8.13 (d, J=7.6 Hz, 1H), 7.70-7.61 (m, 3H), 7.38-7.33 (m, 1H), 6.96-6.89 (m, 3H), 6.16 (d, J=2.8 Hz, 1H), 3.86 (br s, 2H), 2.99-2.78 (m, 2H). LCMS: m/z 305.28 [M+H]⁺.

### Compound 107, N-(2-(1H-imidazol-4-yl)ethyl)-2-(1H-pyrrol-3-yl)quinazolin-4-amine (22%)

¹H NMR (400 MHz, DMSO-d₆): δ 11.80 (br s, 1H), 11.04 (s, 1H), 8.14 (br s, 1H), 8.10 (d, J=8.0 Hz, 1H), 7.66-7.62 (m, 1H), 7.59-7.54 (m, 3H), 7.34-7.30 (m, 1H), 6.91 (br s, 1H), 6.78 (q, J=2.4 Hz, 1H), 6.74 (d, J=1.6 Hz, 1H), 3.81 (q, J=6.8 Hz, 2H), 2.94 (br s, 2H). LCMS: m/z 305.25 [M+H]⁺.

### Compound 109, 3-((2-(1H-pyrrol-2-yl)quinazolin-4-yl)amino)propan-1-ol (29%)

¹H NMR (400 MHz, DMSO-d₆): δ 11.31 (s, 1H), 8.15-8.10 (m, 2H), 7.70-7.66 (m, 1H), 7.61 (dd, J=8.4 Hz, 0.8 Hz, 1H), 7.37-7.32 (m, 1H), 6.90-6.87 (m, 2H), 6.16-6.13 (m, 1H), 4.57 (t, J=5.2 Hz, 1H), 3.71 (q, J=6.4 Hz, 2H), 3.56 (q, J=6.8 Hz, 2H), 1.85 (quin, J=6.8 Hz, 2H). LCMS: m/z 269.35 [M+H]⁺.

### Compound 110, 3-((2-(1H-pyrrol-3-yl)quinazolin-4-yl)amino)propan-1-ol (30%)

¹H NMR (400 MHz, DMSO-d₆): δ 11.03 (s, 1H), 8.11 (d, J=8.0 Hz, 1H), 8.00 (t, J=5.6 Hz, 1H), 7.64 (t, J=7.6 Hz, 1H), 7.58 (d, J=7.6 Hz, 1H), 7.34-7.30 (m, 1H), 6.78-6.76 (m, 1H), 6.72 (d, J=1.2 Hz, 1H), 4.59 (br s, 1H), 3.66 (q, J=6.4 Hz, 2H), 3.54 (t, J=6.4 Hz, 2H), 1.86 (quin, J=6.4 Hz, 2H). LCMS: m/z 269.41 [M+H]⁺.

### Example 2—Activity of Anti-Tropomyosin Compounds as Monotherapy

The ability of compounds of the invention to inhibit the proliferation of cancer cells representative of non-small cell lung cancer and ovarian cancer (Table 2), pancreatic cancer, colorectal cancer and prostate cancer (Table 3) was assessed. Briefly, a predetermined number of cells as calculated from cell growth assays for each of the cell lines employed were seeded into their respective culture medium (using ATCC culture parameters—http://www.atcc.org) and cultured for 24 h at 37° C. and 5% CO₂ in 96-well culture plates. Once attached, each cell line was then exposed to various concentrations of each respective analogue (30, 10, 3, 1, 0.3 and 0.1 μM), cultured for a further 72 h and exposed to Cell-Titre luminescent reagent (100 μL/well) for a further 30 min. Luminescence was captured using an EnVision multilabel reader and the data for each analogue concentration compared against no treatment control. Cell viability was normalised to control (vehicle alone) and dose response curves, and half maximal effective concentration (EC₅₀) values were determined using Graph Pad Prism 6 (nonlinear regression sigmoidal dose-response variable slope).

TABLE 2
Anti-proliferative activity of compounds of the disclosure
against non-small cell lung cancer and ovarian cancer cells

| | |
|---|---|
| Compound | IC₅₀/μM |

| ID | A549 (NSCLC) | A2780 (ovarian) |
|---|---|---|
| 1 | 8.06 | 8.59 |
| 2 | 11.1 | 10.7 |
| 3 | 5.92 | 8.89 |
| 4 | 8.56 | 8.30 |
| 5 | 2.74 | 0.901 |
| 6 | >30.0 | >30.0 |
| 7 | 10.2 | 10.8 |
| 8 | >30.0 | >30.0 |
| 9 | 4.94 | 5.79 |
| 10 | >30.0 | >30.0 |
| 11 | 10.3 | 6.43 |
| 12 | 8.24 | 10.2 |
| 13 | 6.50 | 5.65 |
| 14 | >30.0 | >30.0 |
| 15 | >30.0 | >30.0 |
| 16 | >30.0 | 22.5 |
| 17 | >30.0 | 22.7 |
| 18 | >30.0 | >30.0 |
| 19 | >30.0 | >30.0 |
| 20 | >30.0 | >30.0 |
| 21 | >30.0 | >30.0 |
| 22 | >30.0 | >30.0 |
| 23 | >30.0 | >30.0 |
| 24 | >30.0 | >30.0 |
| 25 | >30.0 | >30.0 |
| 26 | 22.0 | 16.9 |
| 27 | 2.84 | 6.74 |
| 28 | >30.0 | >30.0 |
| 29 | 9.21 | 6.91 |
| 30 | >30.0 | >30.0 |
| 31 | 3.61 | 1.21 |
| 32 | 4.63 | 1.28 |
| 33 | 21.0 | 8.95 |
| 34 | 14.1 | 12.8 |
| 35 | >30.0 | 7.47 |
| 36 | 2.67 | 2.32 |
| 37 | 3.82 | 3.82 |
| 38 | 26.8 | 18.8 |
| 39 | >30.0 | 11.9 |
| 40 | 6.27 | 4.34 |
| 41 | 3.06 | 0.855 |
| 42 | 4.94 | 1.69 |
| 43 | 6.09 | 1.03 |
| 44 | 5.80 | 2.49 |
| 45 | 18.4 | 8.55 |
| 46 | 3.04 | 2.34 |
| 47 | 3.28 | 6.13 |
| 48 | 7.40 | 4.52 |
| 49 | 8.77 | 6.87 |
| 50 | 5.30 | 7.68 |
| 51 | 3.07 | 1.39 |
| 52 | 5.60 | 1.19 |
| 53 | >30.0 | 18.2 |
| 54 | 4.65 | 3.80 |
| 55 | 4.45 | 7.16 |
| 56 | 8.50 | 1.01 |
| 57 | 1.53 | 0.655 |
| 58 | 5.85 | 6.22 |
| 59 | 3.70 | 5.54 |
| 60 | 2.74 | 2.21 |
| 61 | 11.9 | 7.09 |
| 62 | 7.50 | 13.1 |
| 63 | 19.8 | 5.67 |
| 64 | 2.87 | 2.38 |
| 65 | 19.4 | 6.17 |
| 66 | >30.0 | 13.7 |
| 67 | 5.90 | 7.59 |
| 68 | 6.65 | 3.91 |
| 69 | 2.74 | 0.797 |
| 70 | 11.7 | 8.33 |
| 71 | 7.14 | 2.88 |
| 72 | 11.2 | 3.43 |
| 73 | 11.7 | 2.13 |
| 74 | 1.70 | 1.16 |
| 75 | 1.49 | 0.467 |
| 76 | 8.88 | 7.11 |
| 77 | 3.04 | 3.49 |
| 78 | 2.33 | 2.95 |
| 79 | >30.0 | 18.9 |
| 80 | 4.00 | 3.33 |
| 81 | 3.10 | 1.20 |
| 82 | 7.46 | 1.88 |
| 83 | 9.16 | 2.20 |
| 84 | 1.39 | 1.51 |
| 85 | 6.81 | 1.74 |
| 86 | 3.23 | 1.07 |
| 87 | 2.36 | 2.60 |
| 88 | 3.42 | 1.74 |
| 89 | 6.1 | 2.73 |
| 90 | 3.87 | 5.19 |
| 91 | 6.76 | 5.42 |
| 92 | 1.30 | 2.22 |
| 93 | 10.1 | 2.65 |
| 94 | 5.57 | 4.59 |
| 95 | 1.76 | 2.78 |
| 96 | 6.70 | 5.52 |
| 97 | 4.99 | 3.07 |
| 98 | 5.21 | 1.03 |
| 99 | 22.2 | 10.3 |
| 100 | 19.4 | 11.8 |
| 101 | 11.6 | 12.5 |
| 102 | 7.45 | 1.32 |
| 103 | 24.1 | 16.5 |
| 104 | >30.0 | >30.0 |
| 105 | 24.1 | 9.05 |
| 106 | 15.6 | 8.43 |
| 107 | 9.40 | 1.95 |
| 108 | 9.53 | 5.79 |
| 109 | 21.1 | 13.0 |
| 110 | 9.19 | 4.87 |
| 111 | >30.0 | >30.0 |
| 112 | 0.938 | 0.767 |
| 113 | 8.79 | 2.54 |
| 114 | 2.36 | 0.937 |
| 115 | 2.52 | 1.24 |
| 116 | 3.60 | 1.39 |
| 117 | 3.05 | 1.62 |
| 118 | 4.05 | 1.92 |
| 119 | 2.46 | 0.759 |
| 120 | 5.28 | 1.40 |
| 121 | 4.00 | 2.38 |
| 122 | 3.17 | 2.68 |
| 123 | 3.36 | 2.38 |
| 124 | 2.25 | 2.74 |
| 125 | 1.62 | 0.968 |
| 126 | 14.8 | 2.50 |
| 127 | 2.54 | 2.62 |
| 128 | 0.943 | 0.606 |
| 129 | 1.00 | 0.316 |
| 130 | 10.9 | 2.88 |
| 131 | 11.5 | 3.00 |
| 132 | 8.03 | 4.71 |
| 133 | 14.3 | 7.17 |
| 134 | 8.86 | 2.68 |
| 135 | 3.19 | 0.660 |
| 136 | 4.77 | 1.03 |
| 137 | 13.4 | 8.71 |
| 138 | 6.07 | 1.10 |
| 139 | 3.44 | 1.10 |
| 140 | 13.2 | 7.26 |
| 141 | 3.30 | 1.22 |
| 142 | >30.0 | >30.0 |
| 143 | 9.48 | 3.01 |
| 144 | 27.8 | 9.37 |
| 145 | >30.0 | 10.9 |
| 146 | 15.7 | 14.1 |
| 147 | 3.18 | 2.81 |
| 148 | >30.0 | 7.77 |
| 149 | 3.51 | 5.04 |
| 150 | 16.5 | 12.2 |
| 151 | 7.41 | 5.81 |

TABLE 3
Anti-proliferative activity of compounds 57, 87 and 92 against
colorectal cancer, pancreatic cancer and prostate cancer cells

| | IC₅₀/μM | | |
|---|---|---|---|
| Compound | Caco-2 | MIA PaCa-2 | PC3 |
| ID | (colorectal) | (pancreatic) | (prostate) |
| 57 | 1.12 | 1.02 | 1.48 |
| 87 | 4.33 | 2.75 | 4.12 |
| 92 | 2.48 | 3.19 | 4.49 |

The ability of compounds of the disclosure to inhibit the proliferation of SK-N-SH neuroblastoma cells was also assessed. The SK-N-SH cell line was maintained as a monolayer in Dulbeccos Modified Eagles medium (DMEM) supplemented with 10% foetal bovine serum (FBS) and grown at 37° C. in a humidified atmosphere with 5% CO₂. For the cytotoxicity assay, SK-N-SH cells were seeded at a density of 2×10³ cells per well in 96-well plates. The cells were treated with serial dilutions of the test compounds (1:2 starting concentration 100 μM) and viability measured after 72 hours using a standard MTS assay. Cell viability was normalised to control (vehicle alone) and dose response curves and IC₅₀ values (Table 4) were determined using Graph Pad Prism 6.

TABLE 4
Anti-proliferative activity of compounds of the
disclosure against SK-N-SH neuroblastoma cells

| Compound ID | IC₅₀/μM |
|---|---|
| 5 | 1.87 |
| 11 | 9.11 |
| 13 | 1.72 |
| 31 | 14.3 |
| 32 | 4.09 |
| 41 | 2.22 |
| 51 | 1.40 |
| 54 | 1.15 |
| 56 | 3.68 |
| 57 | 1.11 |
| 60 | 2.61 |
| 64 | 1.45 |
| 69 | 2.39 |
| 71 | 2.07 |
| 74 | 1.24 |
| 75 | 1.47 |
| 77 | 2.36 |
| 78 | 1.86 |
| 80 | 2.87 |
| 81 | 3.82 |
| 82 | 4.46 |
| 83 | 3.88 |
| 84 | 1.26 |
| 85 | 4.28 |
| 86 | 2.17 |
| 87 | 1.38 |
| 88 | 2.20 |
| 89 | 3.48 |
| 92 | 2.00 |
| 93 | 4.43 |
| 95 | 2.62 |
| 96 | 8.77 |
| 97 | 8.54 |
| 98 | 3.93 |
| 112 | 0.46 |
| 124 | 0.68 |
| 125 | 0.73 |
| 128 | 0.57 |
| 129 | 0.42 |

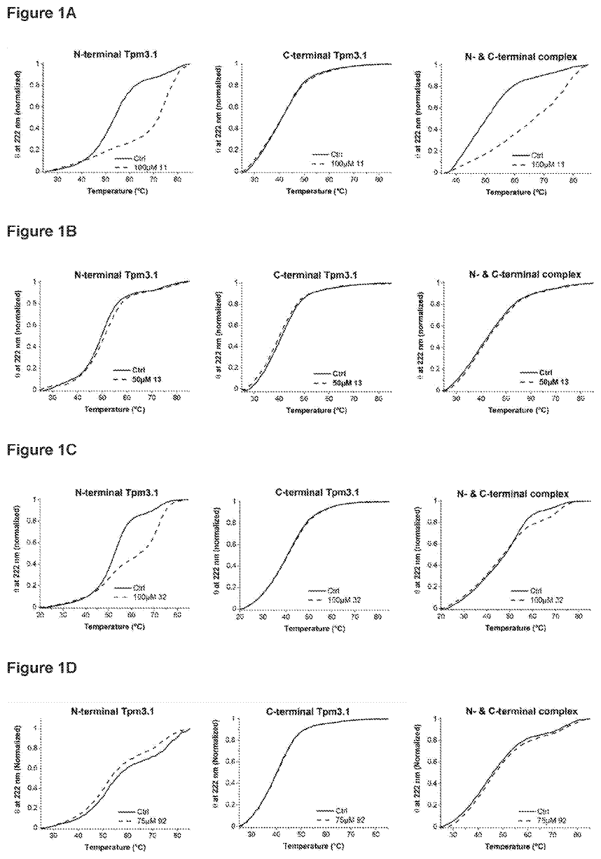

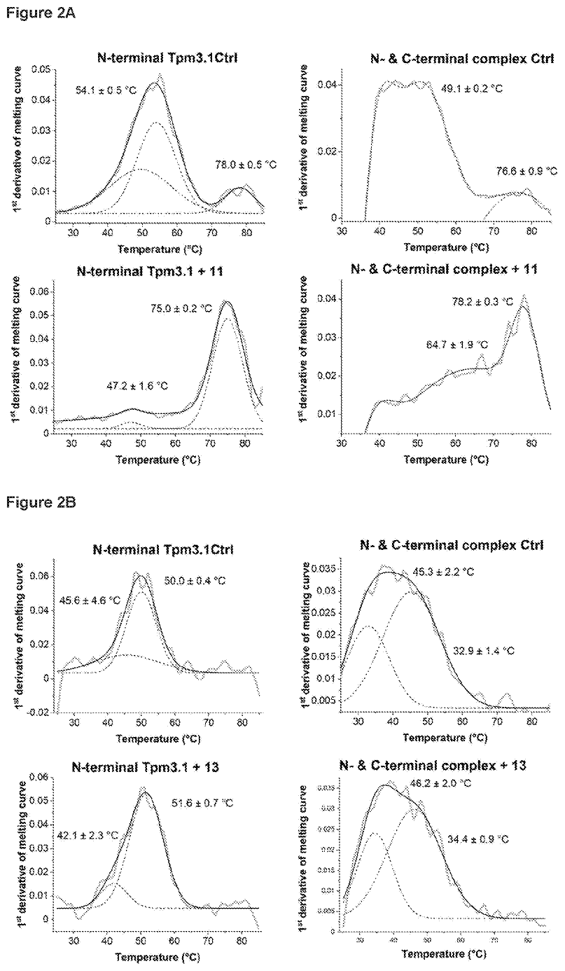

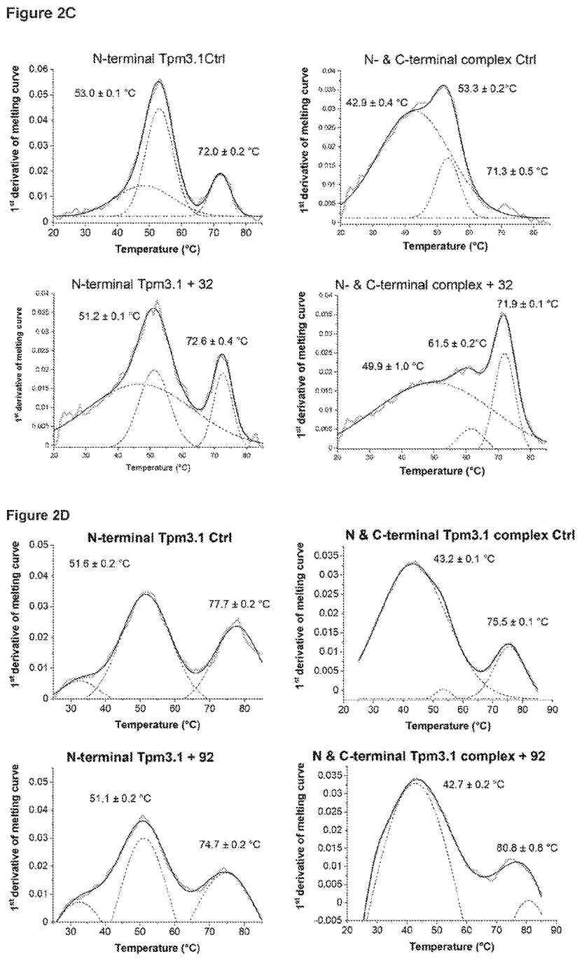

The interactions of compounds 11, 13, 32 and 92 with the N-terminus and overlap region of Tpm3.1 were measured using circular dichroism (CD) spectroscopy (see FIG. 1). Peptides corresponding to the C-terminus (residues 139-248) and N-terminus (residues 2-80 with an initial Ala-Gly dipeptide to mimic N-terminal acetylation) of Tpm3.1 were prepared as described in the literature (Janco et al., 2019). CD spectra and thermal unfolding isotherms of N_82AA_Tpm3.1 and C_109AA_Tpm3.1 peptides were acquired on a Chirascan Plus CD Spectrometer (Applied Photophysics Limited, U.K.) in stoppered 0.5 mm cuvettes (48/Q/0.5; Starna Scientific Ltd.). Proteins were dialyzed O/N against 10 mM NaH₂ PO₄, 150 mM NaCl, 75 μM TCEP (added prior to experiment), 1% (v/v) acetonitrile pH 7 buffer. Final concentration of peptides was 20 μM. Tpm samples containing the test compound (100 μM) were incubated at 37° C. O/N. CD spectra were measured from 195 to 260 nm with a 1 nm step size and a 1.0 mm bandwidth, taking 3 to 4 averages at 37° C. The thermal unfolding was carried at 222 nm over the range 20-85° C. at the rate of 1° C./min. Sample was cooled down to the initial temperature and heating procedure was repeated 2 to 3 times. To obtain information of the major thermal transitions from individual isotherms, data were further smoothed by the Savitzky-Golay method (5-point window), differentiated and then fitted to multiple Gaussian peaks as described previously (Janco et al., 2012) (FIG. 2).

Data demonstrate that compounds 11, 13, 32 and 92 alter the melting profile of the Tpm3.1 N-terminal peptide construct. Compounds 11, 32 and 92 decrease the stability of the N-terminal peptide, while compound 13 has a stabilizing effect. Compounds 11 and 32 also affect the melting profile of the overlap complex formed by the N- and C-terminal peptides, decreasing and increasing stability, respectively. The three compounds have no impact on the melting profile of the C-terminal peptide.

### REFERENCES

Janco, M.; Rynkiewicz, M. J.; Li, L.; Hook, J.; Eiffe, E.; Ghosh, A.; Bocking, T.; Lehman, W. J.; Hardeman, E. C.; Gunning, P. W., Molecular integration of the anti-tropomyosin compound ATM-3507 into the coiled coil overlap region of the cancer-associated Tpm3.1. Sci Rep 2019, 9 (1), 11262.
Janco, M.; Kalyva, A.; Scellini, B.; Piroddi, N.; Tesi, C.; Poggesi, C.; Geeves, M. A., alpha-Tropomyosin with a D175N or E180G mutation in only one chain differs from tropomyosin with mutations in both chains. Biochemistry 2012, 51 (49), 9880-90.

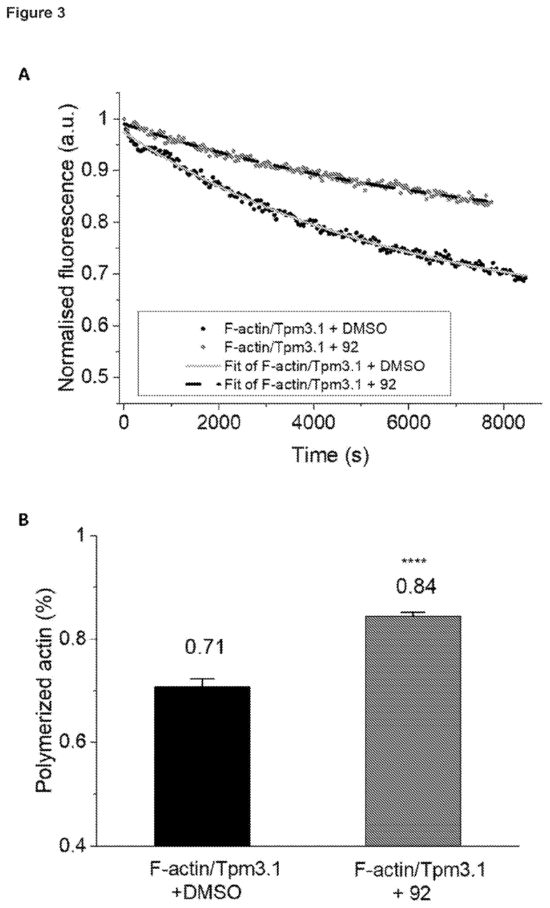

The actin depolymerisation assay was used to confirm the ability of compound 92 to inhibit the ability of Tpm3.1 to protect actin filaments from depolymerisation. A 12 μmol/L solution of labelled F-actin was prepared by polymerising the monomeric actin (35% pyrene labelled Rabbit Muscle (Cytoskeleton Inc)) into filaments in the presence of buffer T (100 mmol/L NaCl; 10 mmol/L Tris HCl pH 7.5; 2 mmol/L MgCl₂; 1 mmol/L EGTA; 0.5 mmol/L DTT) with added ATP (0.2 mmol/L), for 1 hour in the dark at room temperature. Tpm3.1 (10 μmol/L) was reduced in buffer T containing 1 mmol/L DTT at 70° C. for 6 minutes and centrifuged at 60,000 rpm for 30 minutes to remove nonreduced dimers. Prior to the addition to polymerized pyrene-labelled F-actin (3 μmol/L), Tpm3.1 dimers (5 μmol/L) were incubated (overnight, room temperature) with or without 50 μmol/L of the test compound. The F-actin/Tpm3.1±test compound was incubated for one hour at room temperature and samples were then transferred to a black walled 96-well plate. Duplicate samples were diluted 12-fold using an F-actin polymerisation buffer (100 mmol/L NaCl; 10 mmol/L Tris HCl pH 7; 2 mmol/L MgCl₂; 1 mmol/L EGTA; 0.2 mmol/L ATP; 1 mmol/L DTT) and the depolymerisation rates of F-actin alone, F-actin/Tpm3.1, and the F-actin/Tpm3.1/test compound filament complex were measured using a Costar 3915 fluorescence plate reader (407 nm) at 36-second intervals for 160 minutes at room temperature. Data were normalized to the initial fluorescence value and polymerisation curves of duplicate samples were fitted to a two-phase exponential decay model using OriginPro 9.1 (OriginLab) (FIG. 3A). Percent polymerisation was determined at t₁₆₀ as a function of to (100%) (FIG. 3B, p<0.0001).

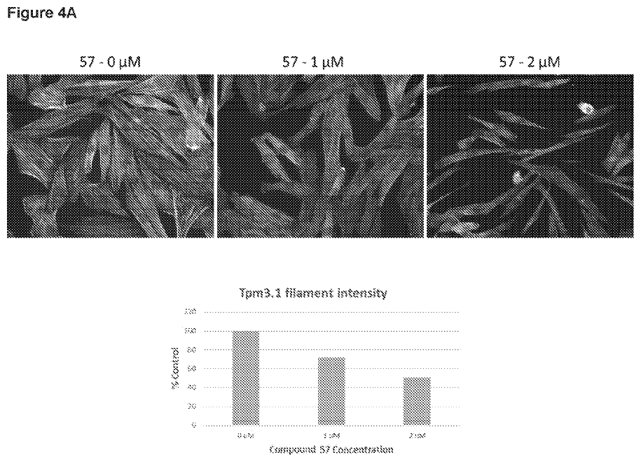

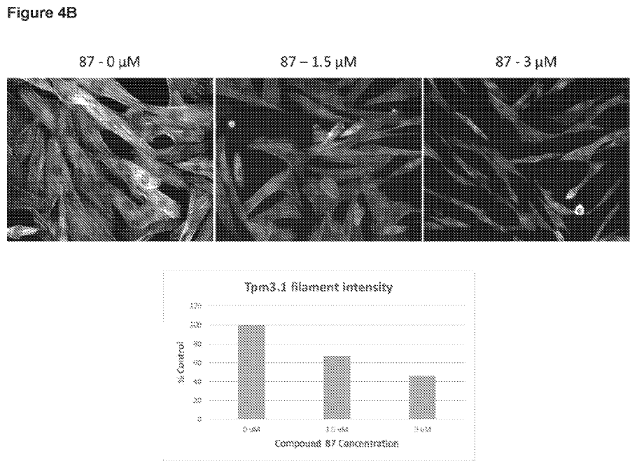

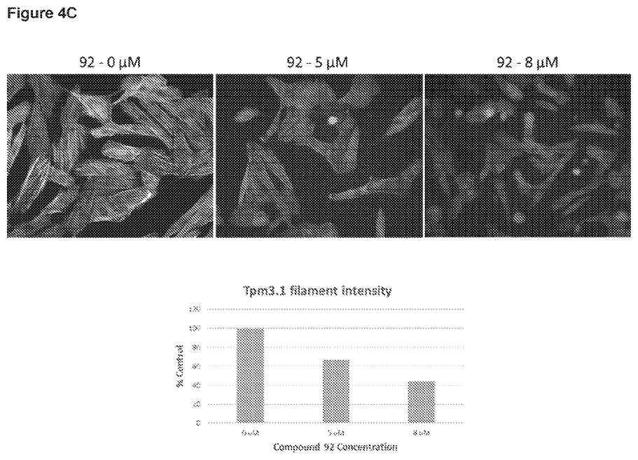

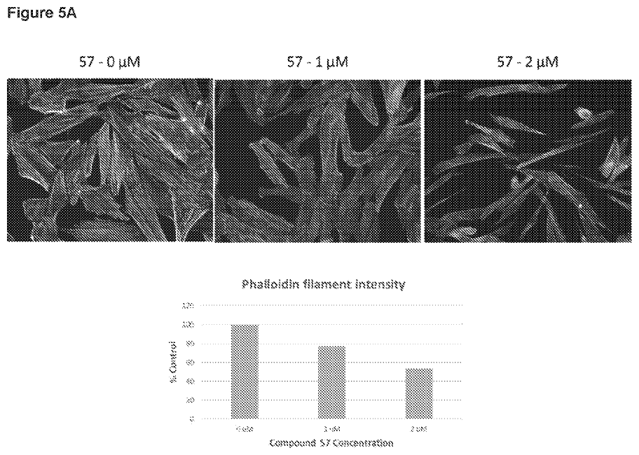

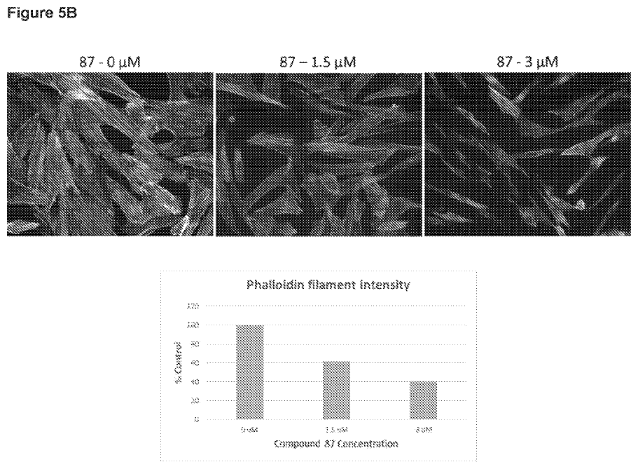

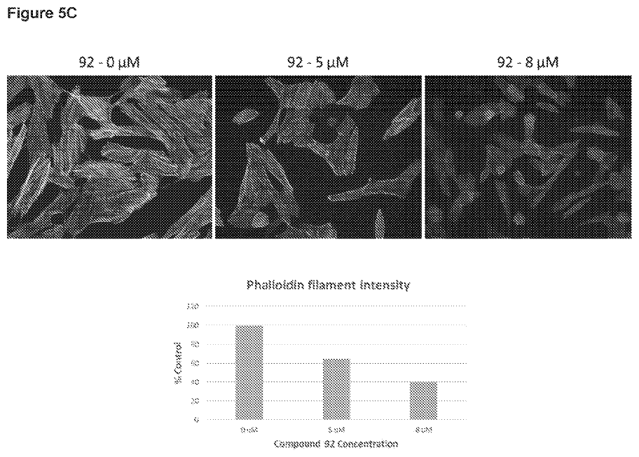

The ability of compounds 57, 87 and 92 to disrupt Tpm3.1-containing actin microfilaments was investigated in SK-N-SH neuroblastoma cells that express Tpm3.1 labelled with the mCherry fluorophore. Cells were seeded at 3×10⁴ cells per well in a volume of 1000 μL complete media onto a 12 well plate containing 13 mm coverslips and left to plate down for 24 hours prior to treatment. Cells were then treated with DMSO and two concentrations of the test compounds. 24 hours post-treatment, cells were fixed with 16% paraformaldehyde (PBS) and stained with 488-Atto-Phalloidin to visualise the actin filament bundles. Single plane images were obtained using the Olympus IX83 epi-fluorescence microscope. Ten fields of view were collected for both Tpm3.1 mCherry fluorophore (FIG. 4) and 488-Atto-Phalloidin (FIG. 5) with each treatment condition.

Quantitation of either Tpm3.1 mCherry filaments or 488-Atto-Phalloidin filaments was performed using ImageJ 1.52p software (ImageJ, NIH). The mean pixel intensity from a cell was measured by using a line across the entire cell cytoplasmic area above or below the nuclear region. The line was made to cross the filament bundles at a 900 angle for the measurement rather than run along a single filament. A total of n=100 cells were measured from ten fields of view. Cells with pixel intensities that were more than twice the average intensity in the field were excluded from the measurement. The mean pixel intensity from n=100 cells was imported into Microsoft Excel to obtain the average intensity of either Tpm3.1 mCherry filaments or 488-Atto-Phalloidin filaments from each treatment condition. Values were normalised as a percent of the 0 μM control in order to generate a bar graph. Data demonstrate that compounds 57, 87 and 92 disrupt Tpm3.1-containing actin microfilaments in a dose-dependent manner.

### Example 3—Activity of Compounds of Formula (I) in Combination with Vinorelbine and Paclitaxel

A study was performed to determine synergistic drug interactions between the cytotoxic agents vinorelbine and paclitaxel in combination with compounds 87 and 92 against the A2780 ovarian cancer cell line.

The A2780 cell line was maintained as a monolayer in Dulbeccos Modified Eagles medium (DMEM) supplemented with 10% foetal bovine serum (FBS) and grown at 37° C. in a humidified atmosphere with 5% CO₂. For the cytotoxicity assay, SK-N-SH cells were seeded at a density of 5×10³ cells per well in 96-well plates. Each screening plate contained one 6×6 dose matrix for two drugs using two-fold dilution steps. Each drug concentration was tested in triplicate using 0.25, 0.5, 1, 2 and 4×IC₅₀ values (87: 3 μM, 92: 5 μM, vinorelbine: 4 nM, paclitaxel: 4 nM). Viability was measured after 72 hours using a standard MTS assay.

Synergistic drug interactions were determined for each pairwise drug combination by applying the Bliss-independence model that assumes independence of drug mechanisms. Prior to the analysis, percent cell viability (CV) for combination data were converted to fractional cell growth inhibition (GI) by the formula:

GI=1−(CV/100)

The Bliss additivism model was used to calculate a predicted combined response C to two single agents with responses A and B (using GI values), as follows:

C=A+B−(A×B)

Experimentally observed values in excess of the predicted additive value for each combination demonstrated a synergistic effect while values that were lower than the predicted additive value demonstrated antagonism of inhibition of cell viability relative to single agent alone. The final score for any combination was reported as the difference between the value predicted by the additive model and the experimentally observed value, normalised to 100. Using GI values the maximum possible synergy score is 100, with 0 indicating no synergy and negative indicating possible antagonism. Scores were also reported as a “Max synergy” score, which is the highest individual synergy score for each combination matrix, and a “Total synergy” score, which was obtained by summation of each individual synergy score for the matrix.

TABLE 5
Synergy scores for drug combinations
in A2780 ovarian cancer cells

| | |
|---|---|
| Vinorelbine | Paclitaxel |

| Compound | Max. | Total | Max. | Total |
|---|---|---|---|---|
| ID | Synergy | Synergy | Synergy | Synergy |
| 87 | 24.46 | 160.51 | 9.54 | 28.53 |
| 92 | 27.55 | 204.68 | 17.97 | 100.06 |

The citation of any reference herein should not be construed as an admission that such reference is available as prior art to the present application. Further, the reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of an two or more of said steps, features, compositions and compounds.

1. A compound having the following formula (I): 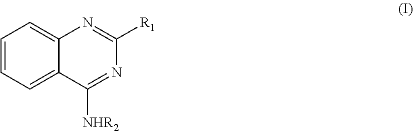
or a pharmaceutically acceptable salt, hydrate, derivative, solvate or prodrug thereof, wherein:
R₁ is selected from the group consisting of:
(i) a heteroaryl group having between 5 and 14 ring atoms in which one or more of the ring atoms are selected from nitrogen, sulfur and oxygen, a heterocyclyl group having between 5 and 14 ring atoms in which one or more of the ring atoms are selected from nitrogen and oxygen,
(ii)
(iii) 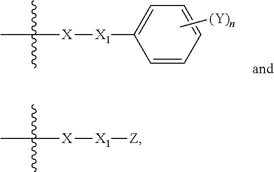
wherein X is O, S or NH;
X₁ is absent or is a straight-chain or branched-chain alkanediyl group having between 1 and 6 carbon atoms;
Y is selected from the group consisting of: CN, NR₅R₆, OH, C₁-C₆alkoxy, halo, CF₃ and C₁-C₆ alkyl;
Z is selected from the group consisting of:a heteroaryl group having 5 or 6 ring atoms in which one or more of the ring atoms are nitrogen, oxygen or sulfur, and (methylenedioxy)phenyl, and wherein the heteroaryl group is optionally substituted with a methyl group;
n is 0, 1, 2 or 3;
R₅ and R₆ are independently selected from the group consisting of: H and C₁-C₆ alkyl;
R₂ is selected from the group consisting of:
(i) T-heteroaryl, wherein the heteroaryl group has 5 ring atoms in which one or more of the ring atoms are nitrogen;
(ii) T-OH
(iii) T-OCH₃
(iv) T-NH₂ and,
(v) 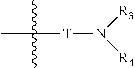
wherein T is a straight-chain or branched-chain alkanediyl group having between 1 and 10 carbon atoms;
R₃ and R₄ are independently selected from the group consisting of: H and C₁-C₆ alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group.

2.-3. (canceled)

4. The compound of claim 1, wherein the heteroaryl group of R₁ has 5 ring atoms in which one or two of the ring atoms are selected from nitrogen, sulfur and oxygen and the heterocyclyl group of R₁ has 5 ring atoms in which one or two of the ring atoms are selected from nitrogen and oxygen.

5. (canceled)

6. The compound claim 1, wherein X is NH.

7.-10. (canceled)

11. The compound of claim 1, wherein X₁ is —CH₂— or —CH₂CH₂—.

12. (canceled)

13. The compound of claim 1, wherein Y is selected from the group consisting of: CN, NR₅R₆, OH, OMe, F, Cl, CF₃ and C₁-C₄ alkyl.

14.-15. (canceled)

16. The compound of claim 1, wherein Z is thienyl, methylpyrrolyl, pyrrolyl, furanyl, pyridyl or (methylenedioxy)phenyl.

17. (canceled)

18. The compound of claim 1, wherein n is 0 or 1.

19.-30. (canceled)

31. The compound of claim 1, wherein R₁ is thienyl, pyrrolyl, furanyl, pyrrolidinyl, or

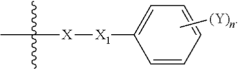

32.-37. (canceled)

38. The compound of claim 1, wherein R₁ is selected from the group consisting of:

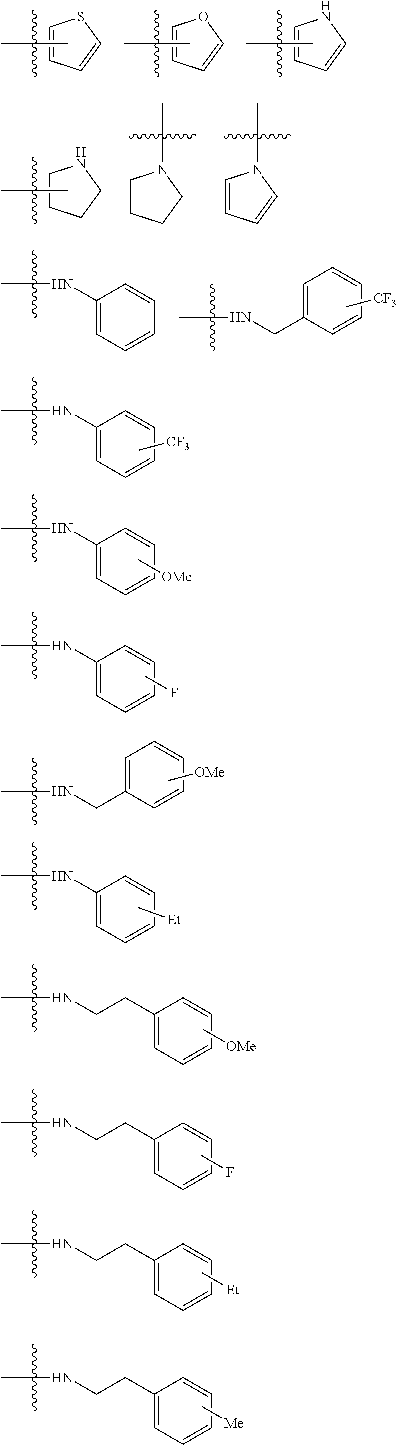

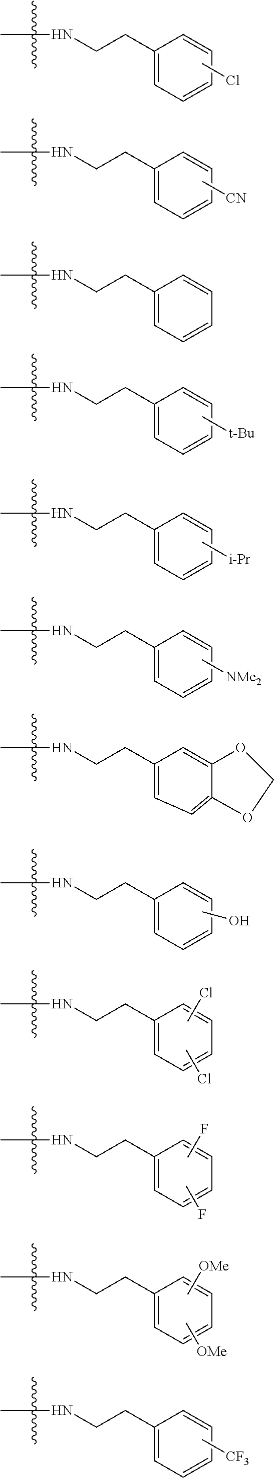

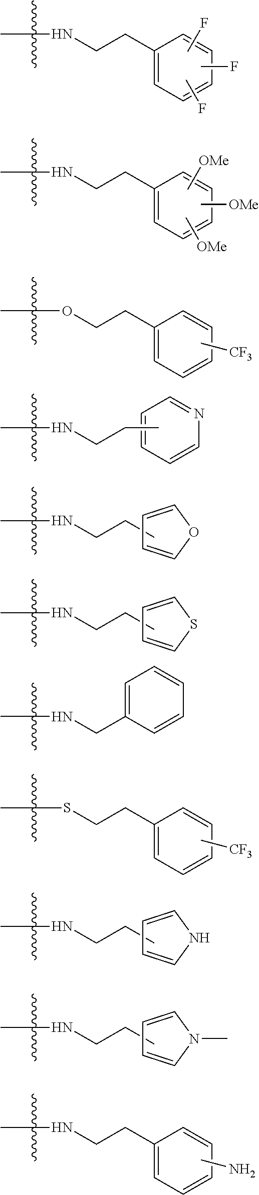

39.-46. (canceled)

47. The compound of claim 1, wherein R₃ and R₄ are independently selected from the group consisting of: H and methyl, or together with the nitrogen to which they are attached form a saturated 5- or 6-membered ring selected from:

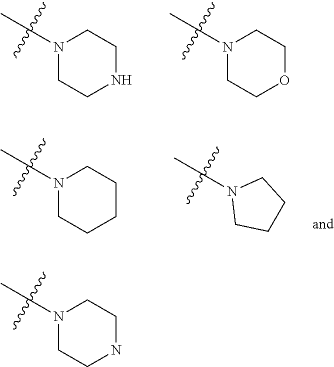 [SMILES: *N1CCNCC1 ; *N1CCOCC1 ; *N1CCCCC1 ; *N1CCCC1 ; *N1CCNCC1]

48.-55. (canceled)

56. The compound of claim 1, wherein R₂ is 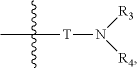
wherein R₃ and R₄ are independently selected from the group consisting of: H and methyl, or together with the nitrogen to which they are attached form a saturated 5- or 6-membered ring selected from:

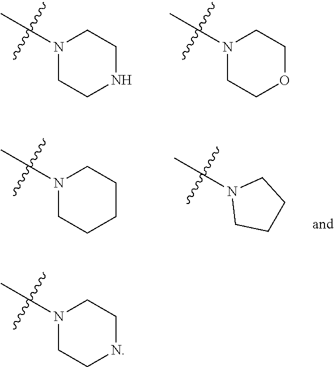

57.-59. (canceled)

60. The compound of claim 1, wherein T is a straight-chain or branched-chain alkanediyl group having 1 or 2 carbon atoms.

61. The compound of claim 1, wherein R₂ is selected from the group consisting of:

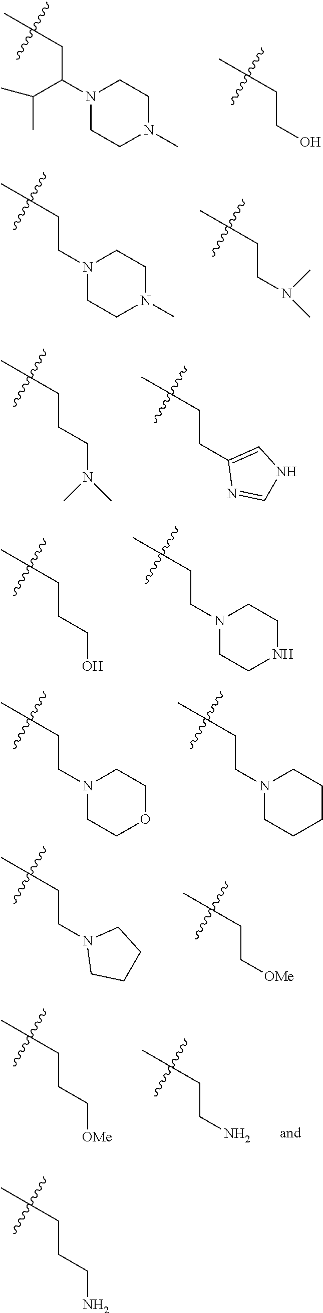

62.-65. (canceled)

66. The compound of claim 1, wherein R₂ is selected from the group consisting of:

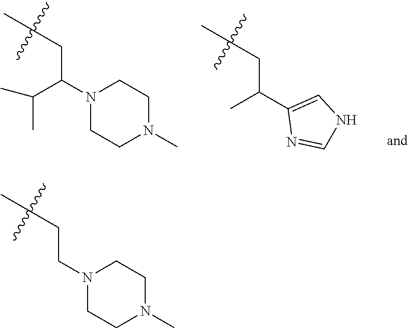

67.-77. (canceled)

78. The compound of claim 1, wherein R₁ is selected from the group consisting of: 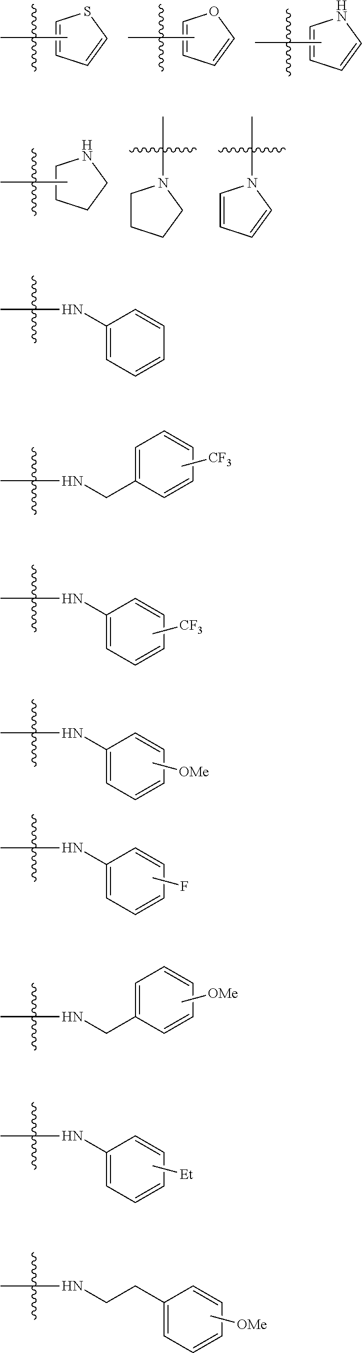 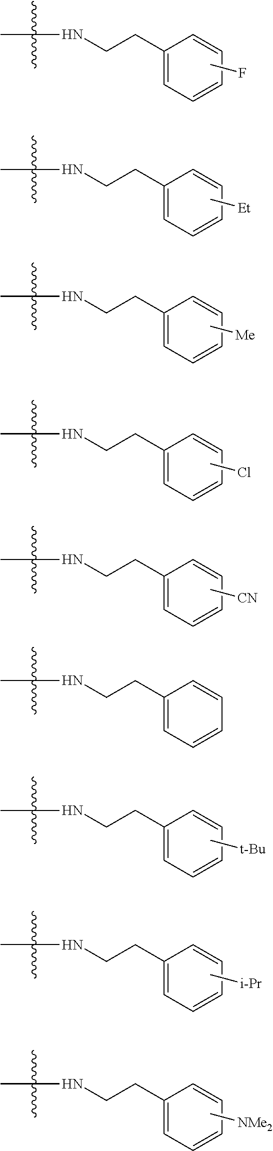 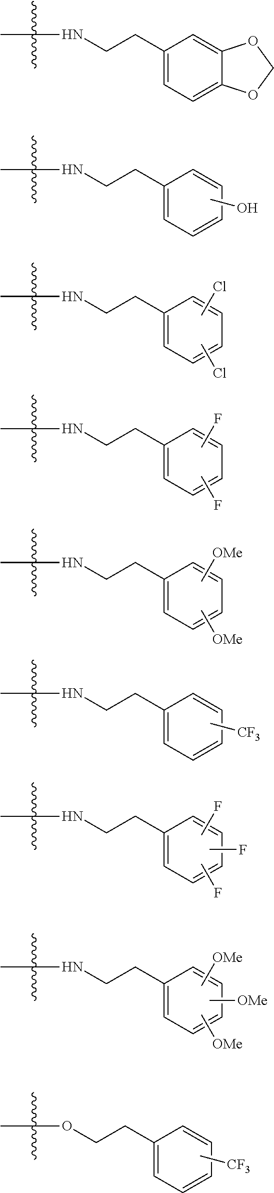 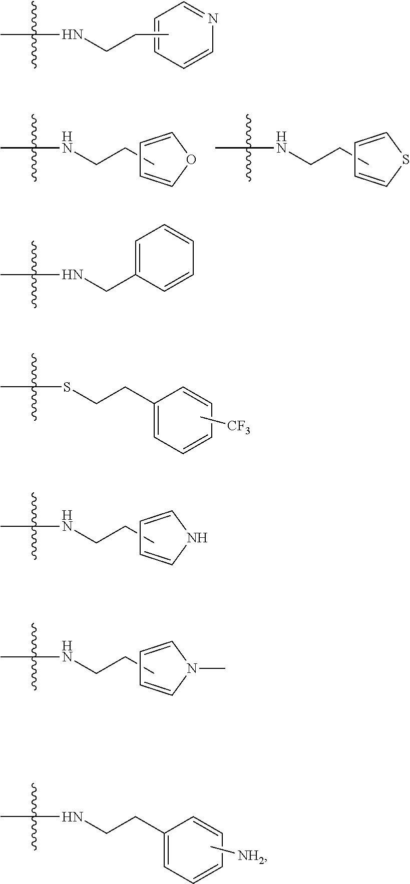
and R₂ is selected from the group consisting of:

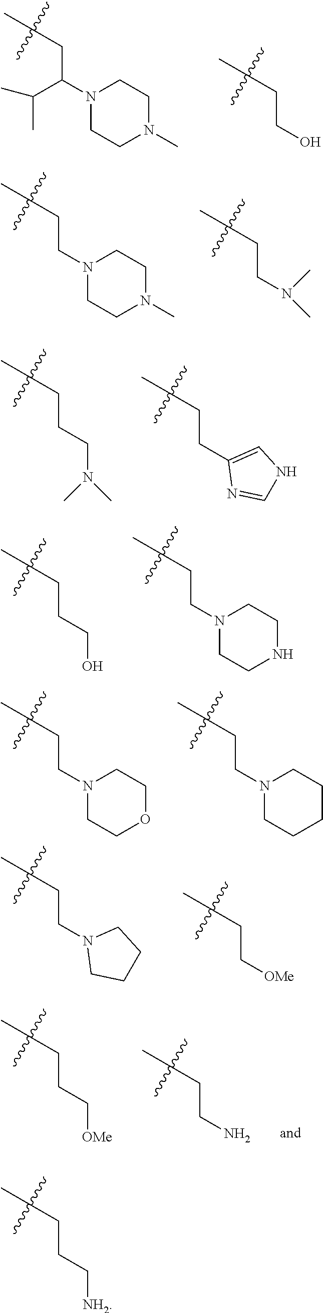

79. A compound of formula (I) selected from the group consisting of:

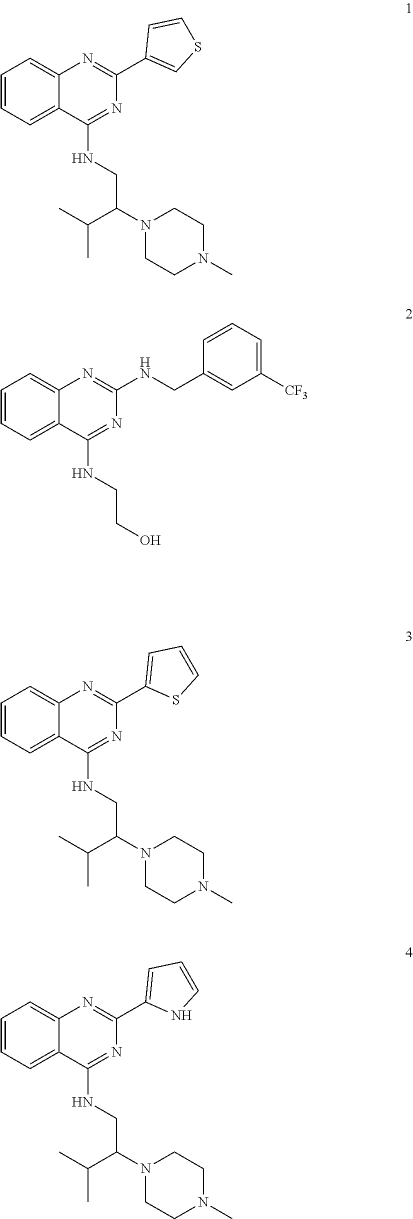 [SMILES: CC(C)C(CNc1nc(-c2ccsc2)nc2ccccc12)N1CCN(C)CC1 ; OCCNc1nc(NCc2cccc(C(F)(F)F)c2)nc2ccccc12 ; CC(C)C(CNc1nc(-c2cccs2)nc2ccccc12)N1CCN(C)CC1 ; CC(C)C(CNc1nc(-c2ccc[nH]2)nc2ccccc12)N1CCN(C)CC1]

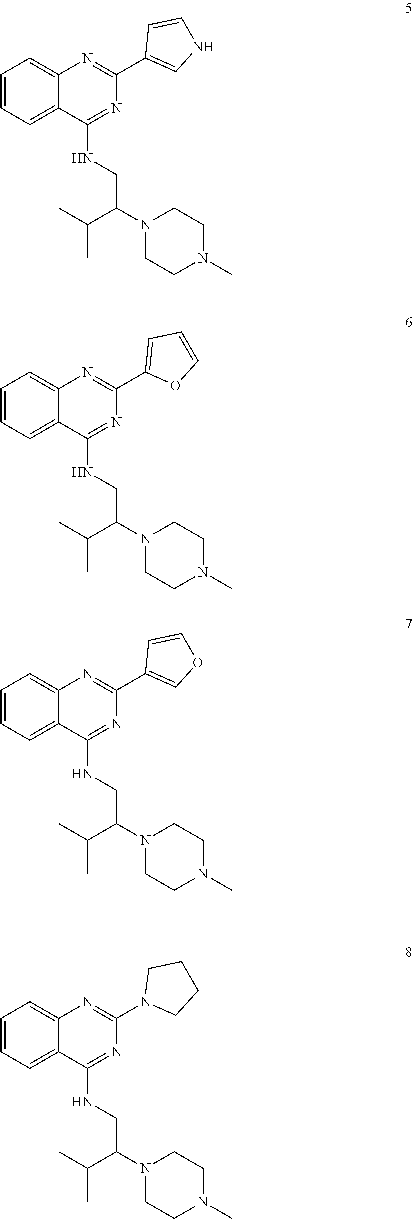

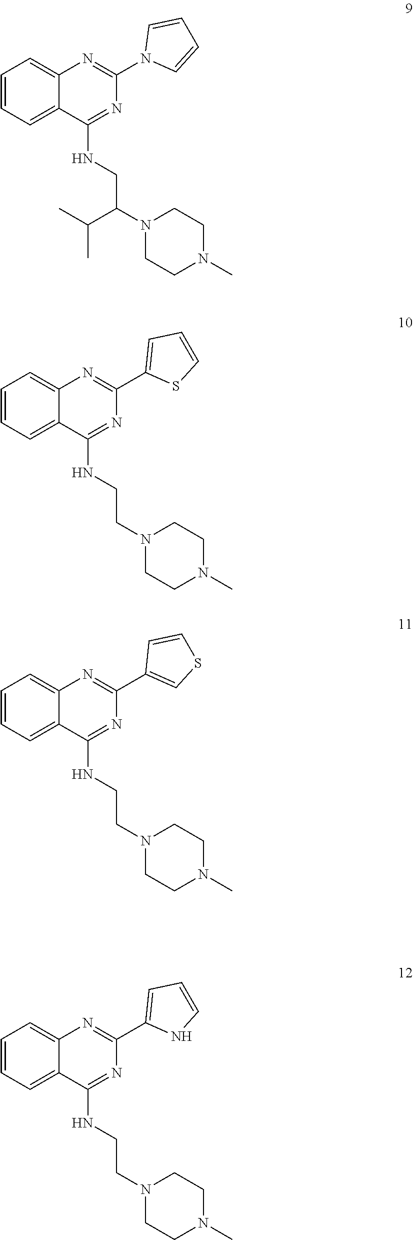

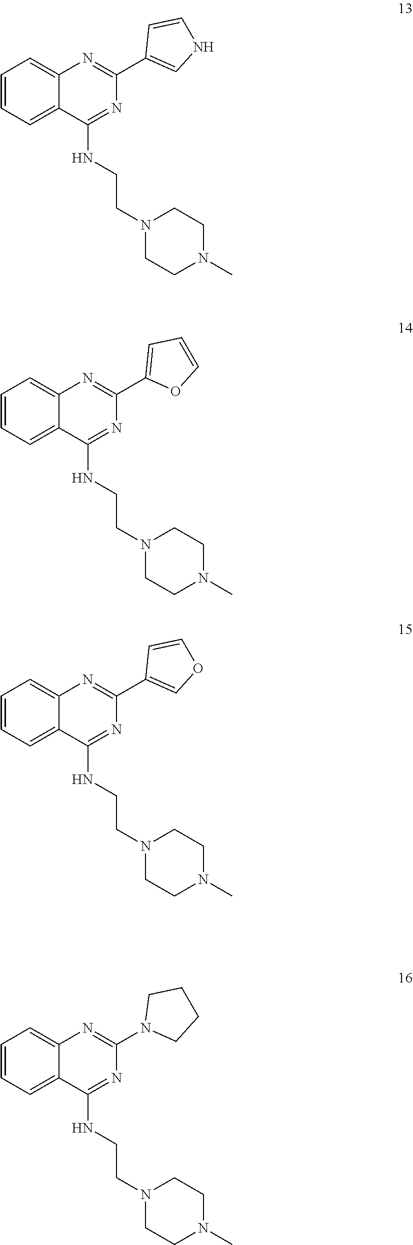

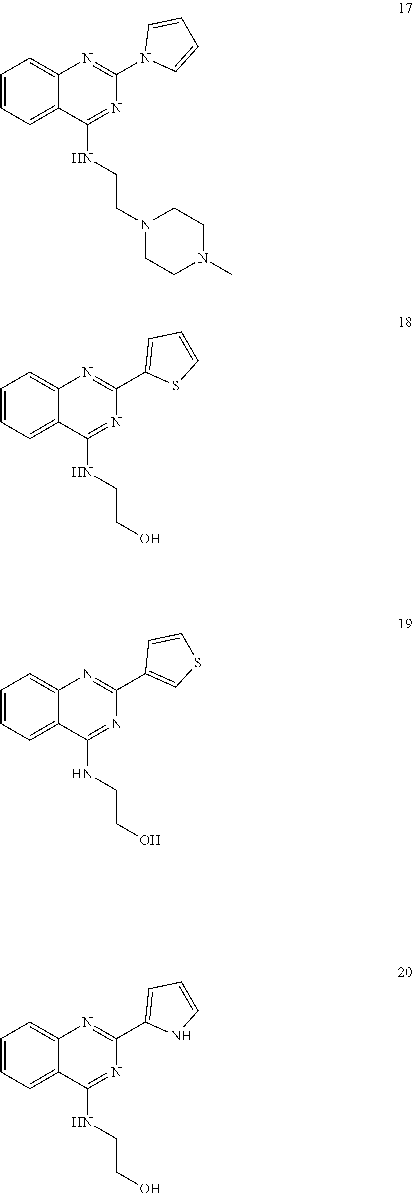 [SMILES: CN1CCN(CCNc2nc(-n3cccc3)nc3ccccc23)CC1 ; OCCNc1nc(-c2cccs2)nc2ccccc12 ; OCCNc1nc(-c2ccsc2)nc2ccccc12 ; OCCNc1nc(-c2ccc[nH]2)nc2ccccc12]

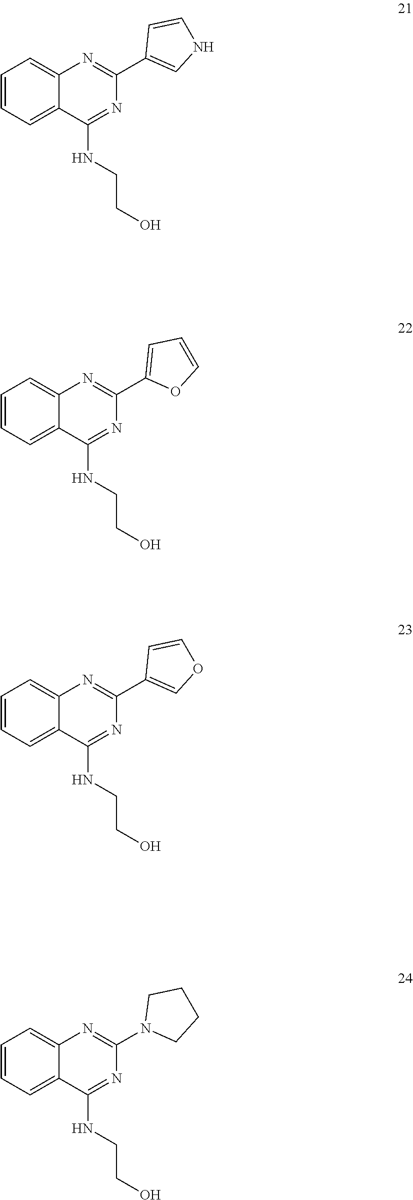 [SMILES: OCCNc1nc(-c2cc[nH]c2)nc2ccccc12 ; OCCNc1nc(-c2ccco2)nc2ccccc12 ; OCCNc1nc(-c2ccoc2)nc2ccccc12 ; OCCNc1nc(N2CCCC2)nc2ccccc12]

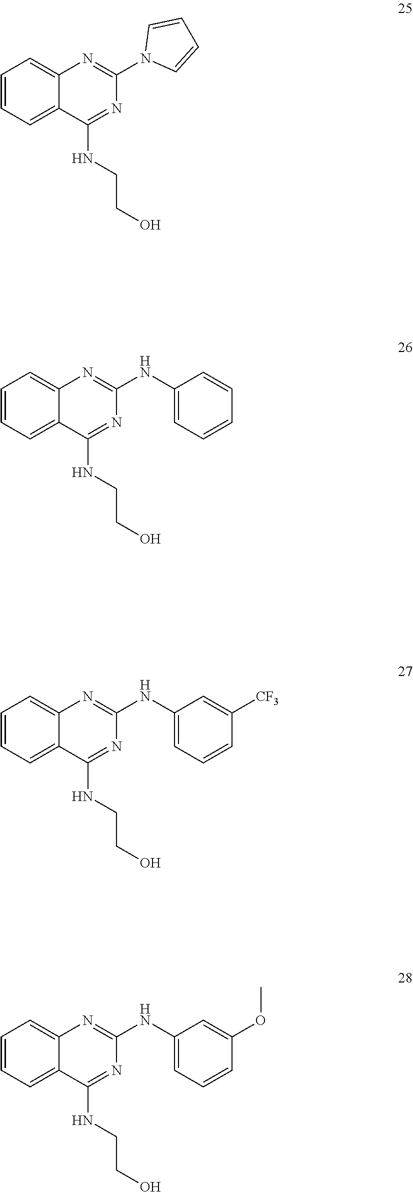 [SMILES: OCCNc1nc(-n2cccc2)nc2ccccc12 ; OCCNc1nc(Nc2ccccc2)nc2ccccc12 ; OCCNc1nc(Nc2cccc(C(F)(F)F)c2)nc2ccccc12 ; COc1cccc(Nc2nc(NCCO)c3ccccc3n2)c1]

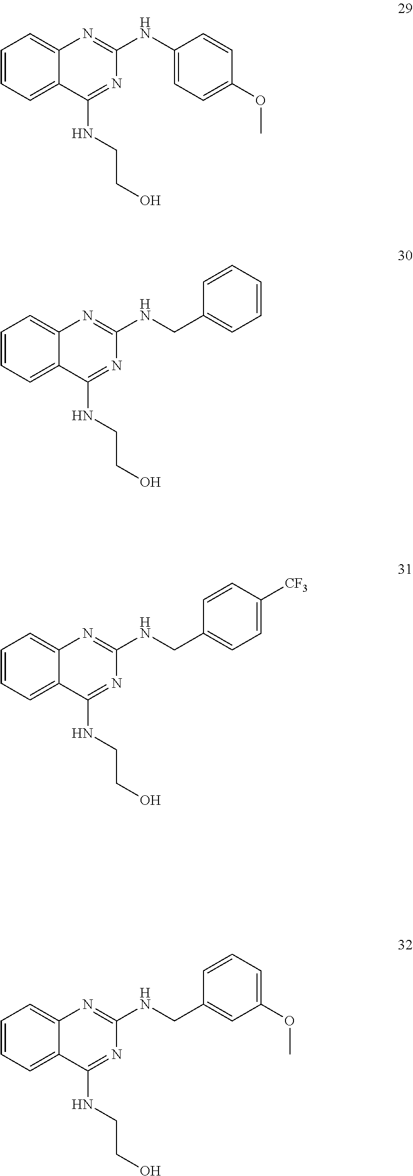 [SMILES: COc1ccc(Nc2nc(NCCO)c3ccccc3n2)cc1 ; OCCNc1nc(NCc2ccccc2)nc2ccccc12 ; OCCNc1nc(NCc2ccc(C(F)(F)F)cc2)nc2ccccc12 ; COc1cccc(CNc2nc(NCCO)c3ccccc3n2)c1]

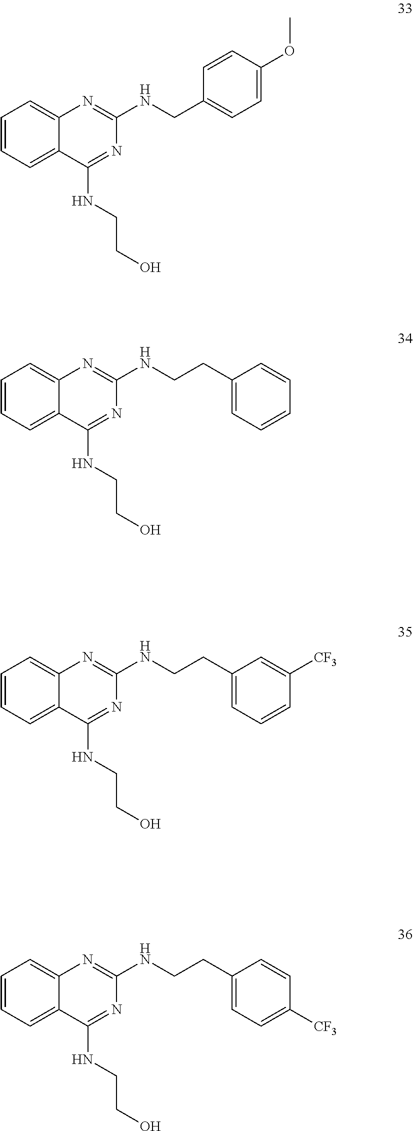 [SMILES: COc1ccc(CNc2nc(NCCO)c3ccccc3n2)cc1 ; OCCNc1nc(NCCc2ccccc2)nc2ccccc12 ; OCCNc1nc(NCCc2cccc(C(F)(F)F)c2)nc2ccccc12 ; OCCNc1nc(NCCc2ccc(C(F)(F)F)cc2)nc2ccccc12]

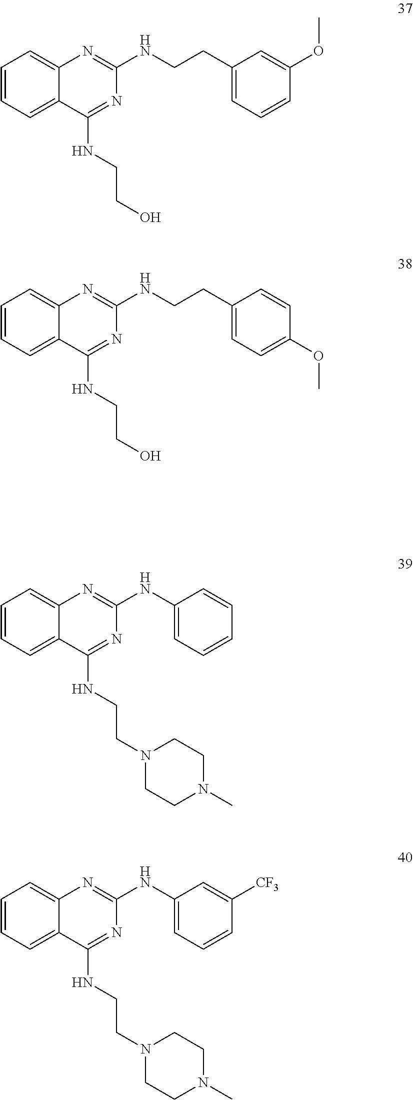 [SMILES: COc1cccc(CCNc2nc(NCCO)c3ccccc3n2)c1 ; COc1ccc(CCNc2nc(NCCO)c3ccccc3n2)cc1 ; CN1CCN(CCNc2nc(Nc3ccccc3)nc3ccccc23)CC1 ; CN1CCN(CCNc2nc(Nc3cccc(C(F)(F)F)c3)nc3ccccc23)CC1]

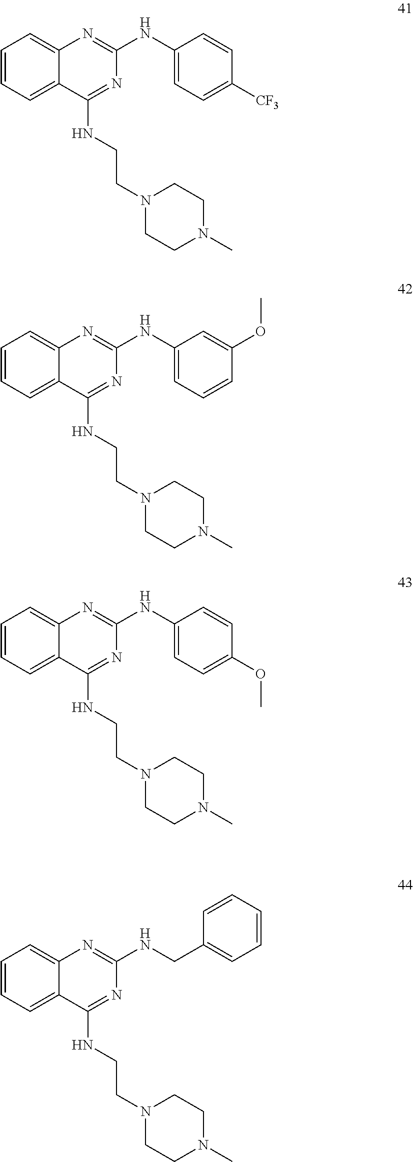 [SMILES: CN1CCN(CCNc2nc(Nc3ccc(C(F)(F)F)cc3)nc3ccccc23)CC1 ; COc1cccc(Nc2nc(NCCN3CCN(C)CC3)c3ccccc3n2)c1 ; COc1ccc(Nc2nc(NCCN3CCN(C)CC3)c3ccccc3n2)cc1 ; CN1CCN(CCNc2nc(NCc3ccccc3)nc3ccccc23)CC1]

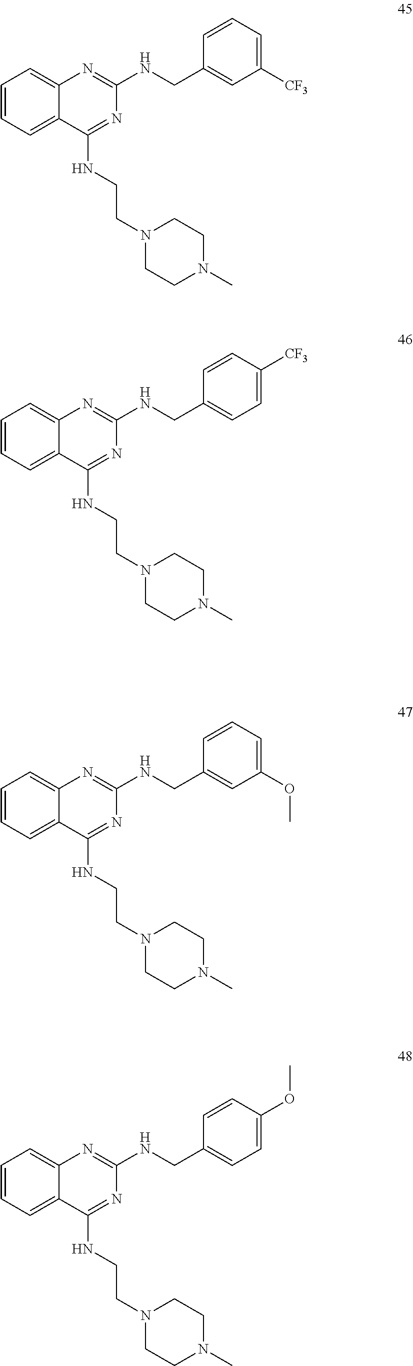 [SMILES: CN1CCN(CCNc2nc(NCc3cccc(C(F)(F)F)c3)nc3ccccc23)CC1 ; CN1CCN(CCNc2nc(NCc3ccc(C(F)(F)F)cc3)nc3ccccc23)CC1 ; COc1cccc(CNc2nc(NCCN3CCN(C)CC3)c3ccccc3n2)c1 ; COc1ccc(CNc2nc(NCCN3CCN(C)CC3)c3ccccc3n2)cc1]

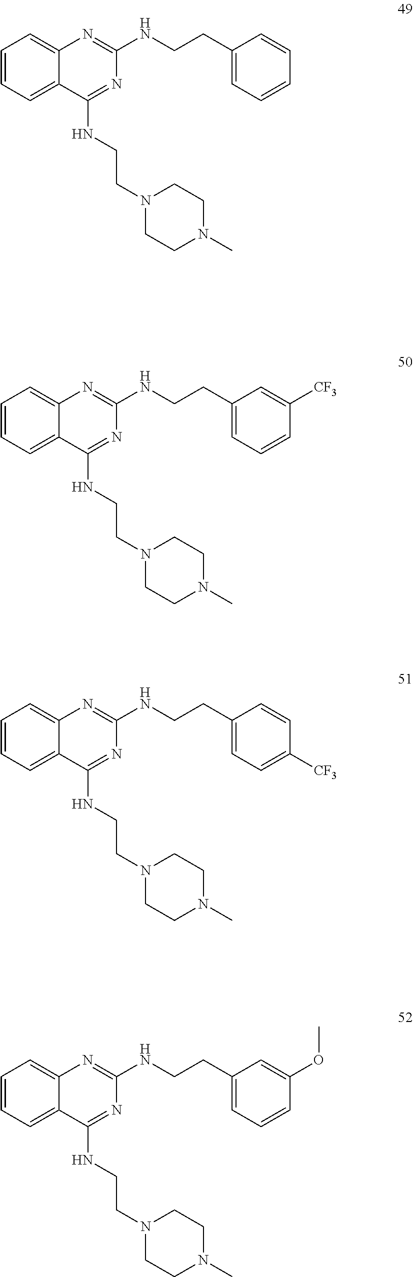 [SMILES: CN1CCN(CCNc2nc(NCCc3ccccc3)nc3ccccc23)CC1 ; CN1CCN(CCNc2nc(NCCc3cccc(C(F)(F)F)c3)nc3ccccc23)CC1 ; CN1CCN(CCNc2nc(NCCc3ccc(C(F)(F)F)cc3)nc3ccccc23)CC1 ; COc1cccc(CCNc2nc(NCCN3CCN(C)CC3)c3ccccc3n2)c1]

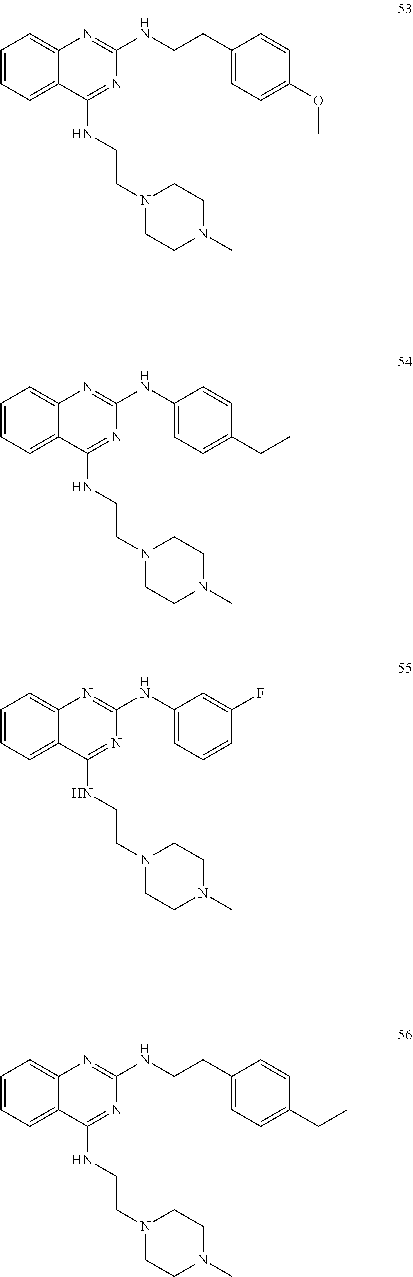 [SMILES: COc1ccc(CCNc2nc(NCCN3CCN(C)CC3)c3ccccc3n2)cc1 ; CCc1ccc(Nc2nc(NCCN3CCN(C)CC3)c3ccccc3n2)cc1 ; CN1CCN(CCNc2nc(Nc3cccc(F)c3)nc3ccccc23)CC1 ; CCc1ccc(CCNc2nc(NCCN3CCN(C)CC3)c3ccccc3n2)cc1]

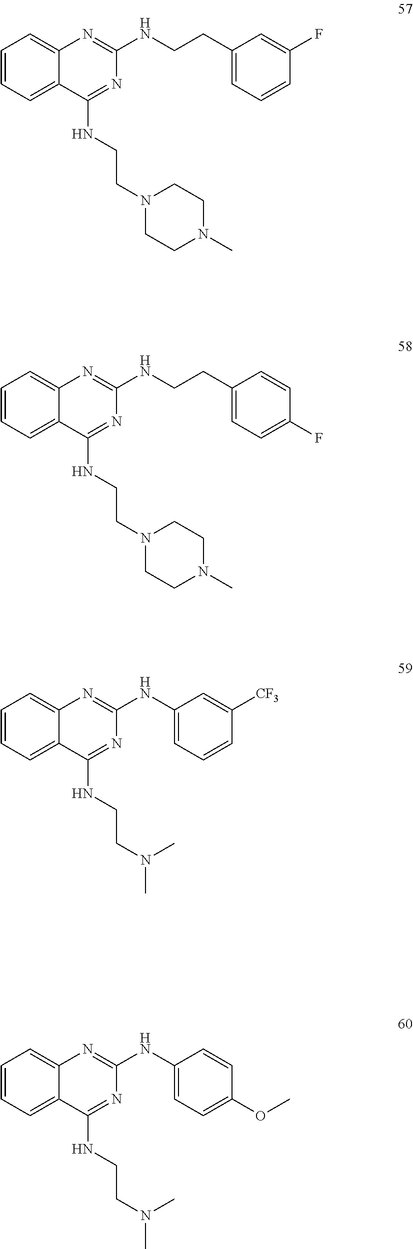 [SMILES: CN1CCN(CCNc2nc(NCCc3cccc(F)c3)nc3ccccc23)CC1 ; CN1CCN(CCNc2nc(NCCc3ccc(F)cc3)nc3ccccc23)CC1 ; CN(C)CCNc1nc(Nc2cccc(C(F)(F)F)c2)nc2ccccc12 ; COc1ccc(Nc2nc(NCCN(C)C)c3ccccc3n2)cc1]

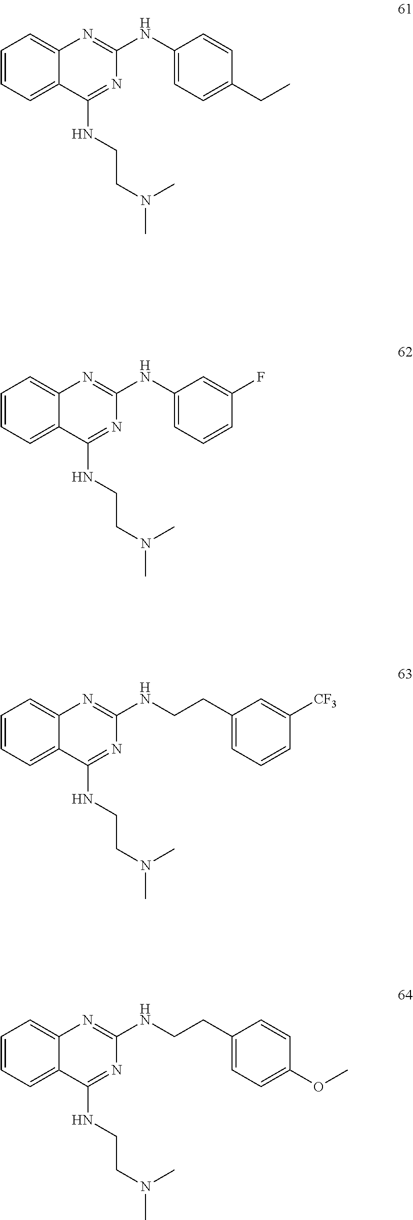 [SMILES: CCc1ccc(Nc2nc(NCCN(C)C)c3ccccc3n2)cc1 ; CN(C)CCNc1nc(Nc2cccc(F)c2)nc2ccccc12 ; CN(C)CCNc1nc(NCCc2cccc(C(F)(F)F)c2)nc2ccccc12 ; COc1ccc(CCNc2nc(NCCN(C)C)c3ccccc3n2)cc1]

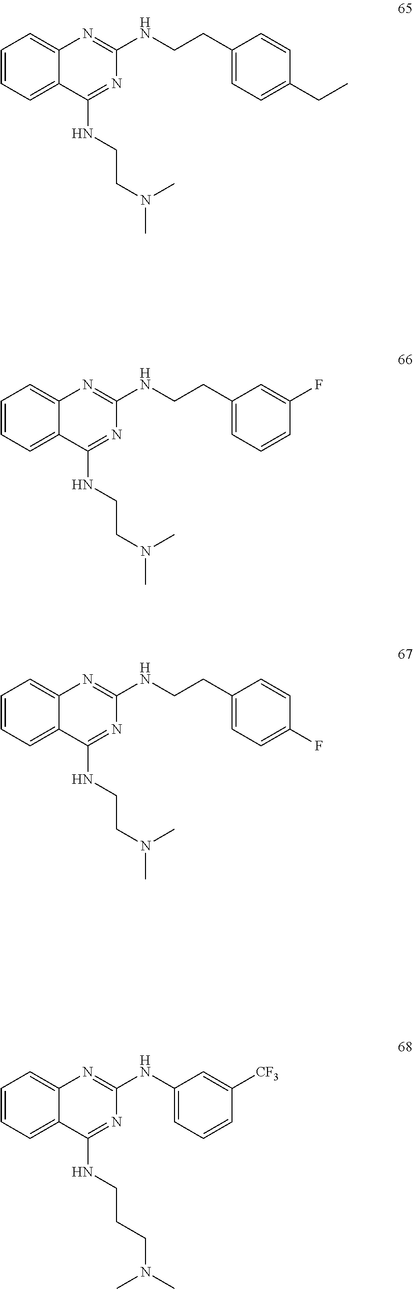 [SMILES: CCc1ccc(CCNc2nc(NCCN(C)C)c3ccccc3n2)cc1 ; CN(C)CCNc1nc(NCCc2cccc(F)c2)nc2ccccc12 ; CN(C)CCNc1nc(NCCc2ccc(F)cc2)nc2ccccc12 ; CN(C)CCCNc1nc(Nc2cccc(C(F)(F)F)c2)nc2ccccc12]

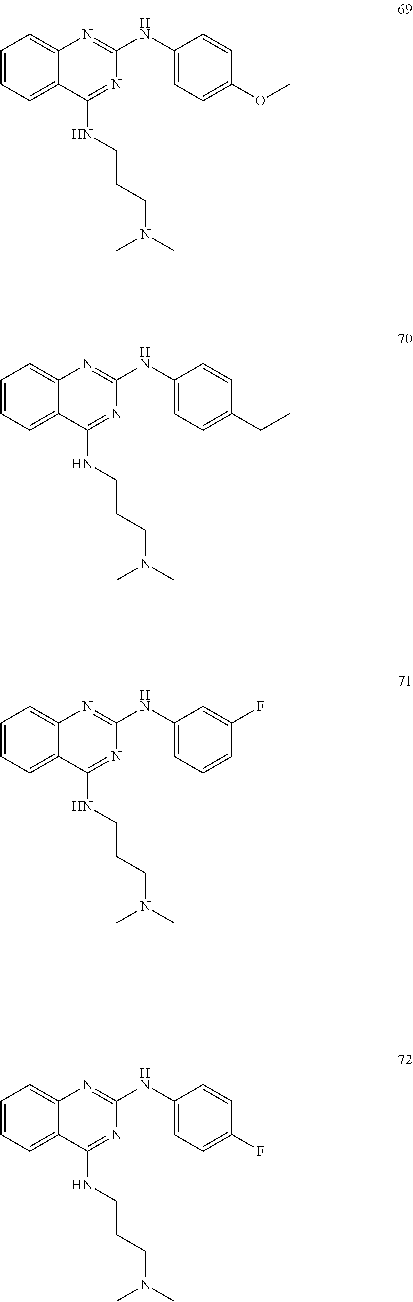 [SMILES: COc1ccc(Nc2nc(NCCCN(C)C)c3ccccc3n2)cc1 ; CCc1ccc(Nc2nc(NCCCN(C)C)c3ccccc3n2)cc1 ; CN(C)CCCNc1nc(Nc2cccc(F)c2)nc2ccccc12 ; CN(C)CCCNc1nc(Nc2ccc(F)cc2)nc2ccccc12]

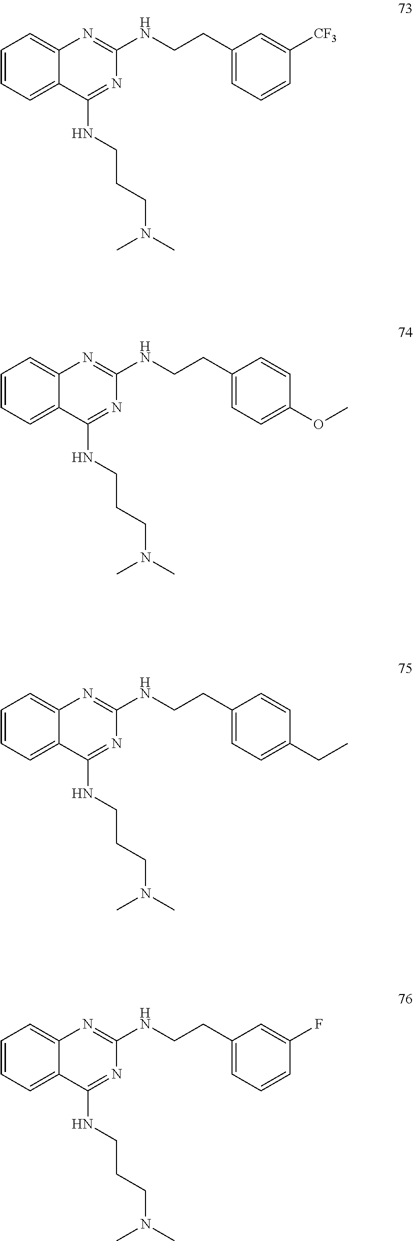 [SMILES: CN(C)CCCNc1nc(NCCc2cccc(C(F)(F)F)c2)nc2ccccc12 ; COc1ccc(CCNc2nc(NCCCN(C)C)c3ccccc3n2)cc1 ; CCc1ccc(CCNc2nc(NCCCN(C)C)c3ccccc3n2)cc1 ; CN(C)CCCNc1nc(NCCc2cccc(F)c2)nc2ccccc12]

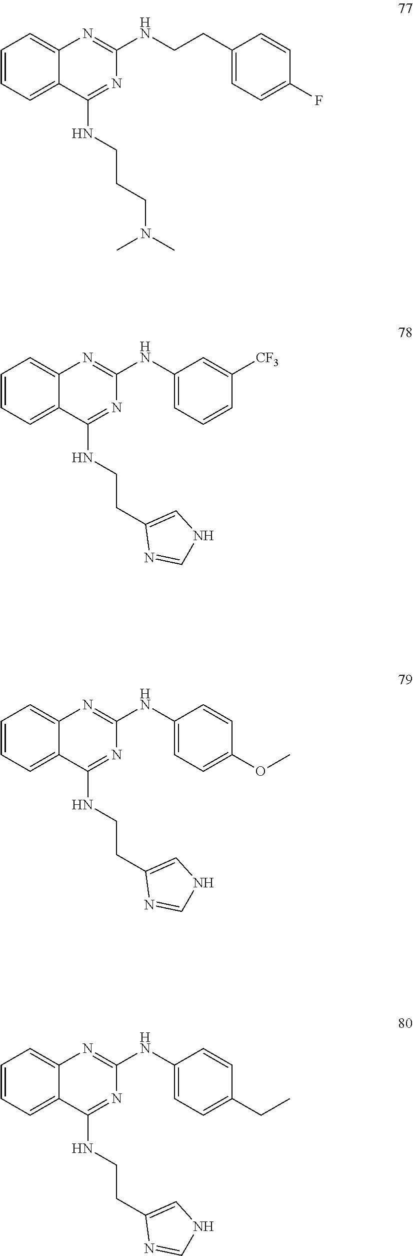 [SMILES: CN(C)CCCNc1nc(NCCc2ccc(F)cc2)nc2ccccc12 ; FC(F)(F)c1cccc(Nc2nc(NCCc3c[nH]cn3)c3ccccc3n2)c1 ; COc1ccc(Nc2nc(NCCc3c[nH]cn3)c3ccccc3n2)cc1 ; CCc1ccc(Nc2nc(NCCc3c[nH]cn3)c3ccccc3n2)cc1]

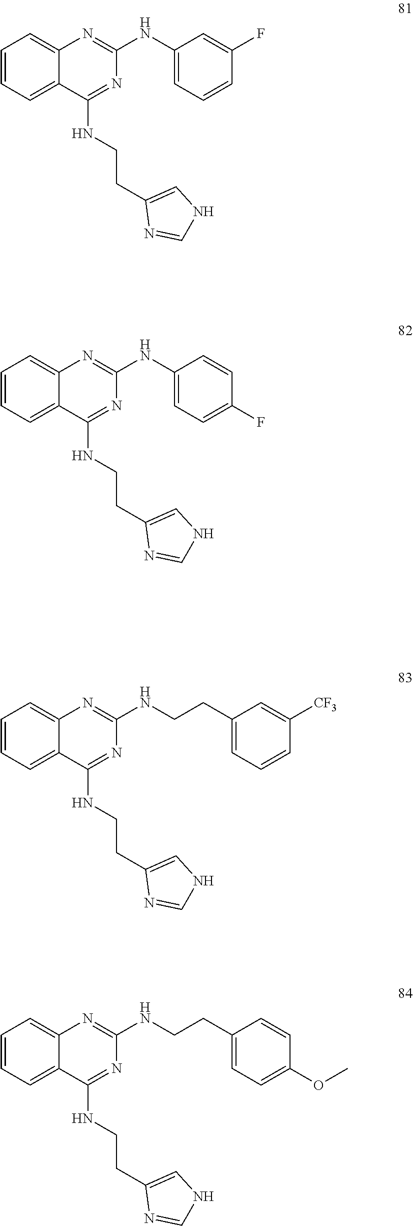 [SMILES: Fc1cccc(Nc2nc(NCCc3c[nH]cn3)c3ccccc3n2)c1 ; Fc1ccc(Nc2nc(NCCc3c[nH]cn3)c3ccccc3n2)cc1 ; FC(F)(F)c1cccc(CCNc2nc(NCCc3c[nH]cn3)c3ccccc3n2)c1 ; COc1ccc(CCNc2nc(NCCc3c[nH]cn3)c3ccccc3n2)cc1]

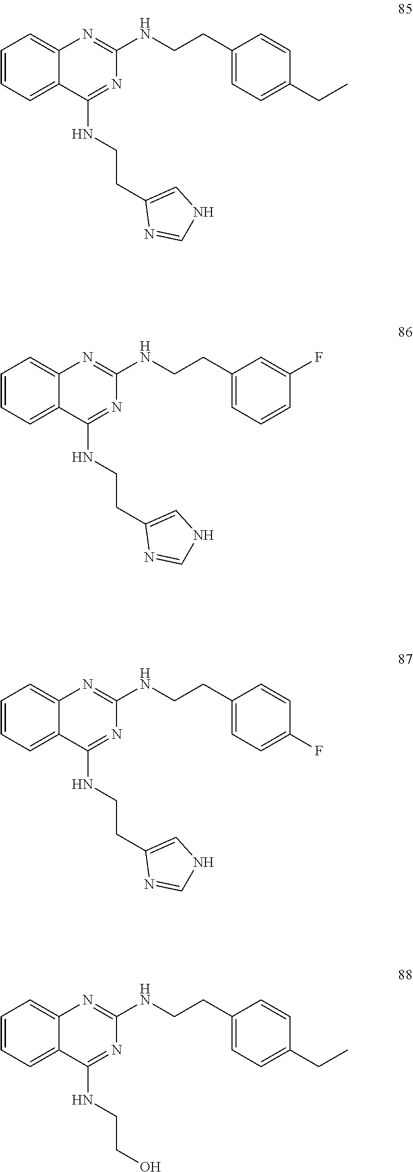 [SMILES: CCc1ccc(CCNc2nc(NCCc3c[nH]cn3)c3ccccc3n2)cc1 ; Fc1cccc(CCNc2nc(NCCc3c[nH]cn3)c3ccccc3n2)c1 ; Fc1ccc(CCNc2nc(NCCc3c[nH]cn3)c3ccccc3n2)cc1 ; CCc1ccc(CCNc2nc(NCCO)c3ccccc3n2)cc1]

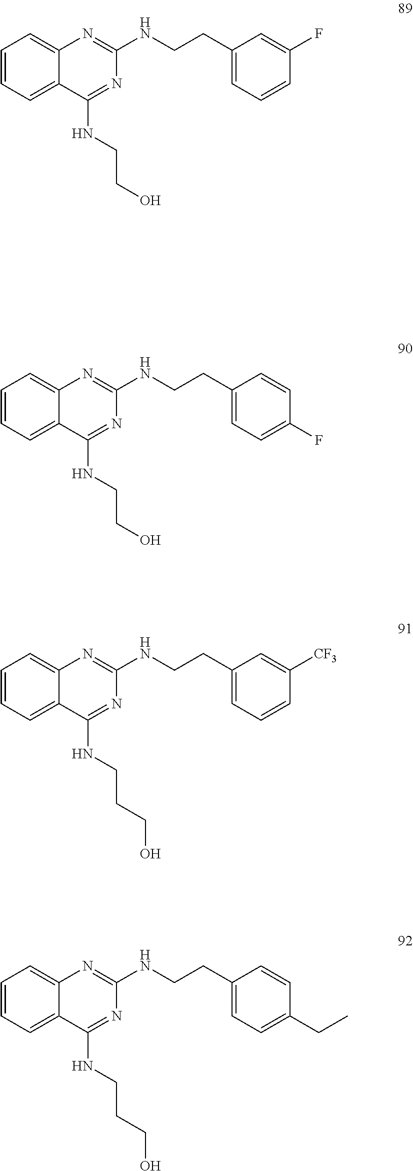 [SMILES: OCCNc1nc(NCCc2cccc(F)c2)nc2ccccc12 ; OCCNc1nc(NCCc2ccc(F)cc2)nc2ccccc12 ; OCCCNc1nc(NCCc2cccc(C(F)(F)F)c2)nc2ccccc12 ; CCc1ccc(CCNc2nc(NCCCO)c3ccccc3n2)cc1]

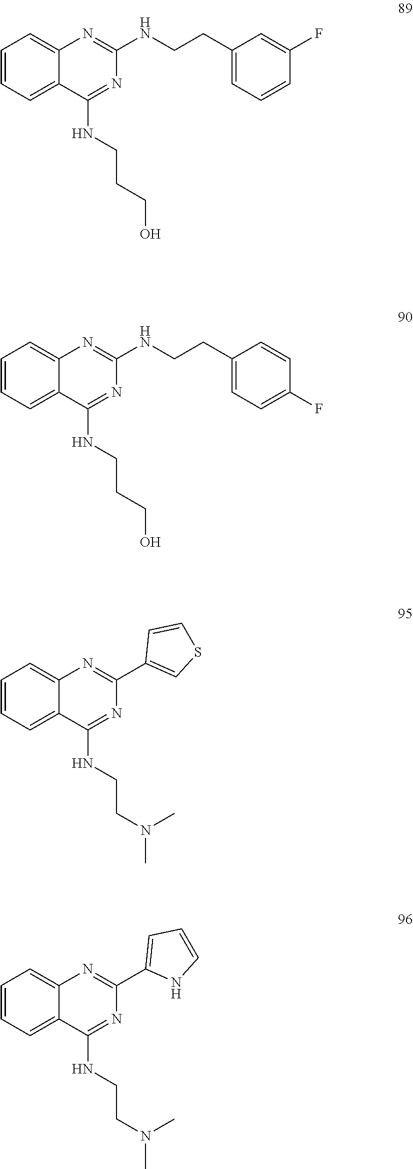 [SMILES: OCCCNc1nc(NCCc2cccc(F)c2)nc2ccccc12 ; OCCCNc1nc(NCCc2ccc(F)cc2)nc2ccccc12 ; CN(C)CCNc1nc(-c2ccsc2)nc2ccccc12 ; CN(C)CCNc1nc(-c2ccc[nH]2)nc2ccccc12]

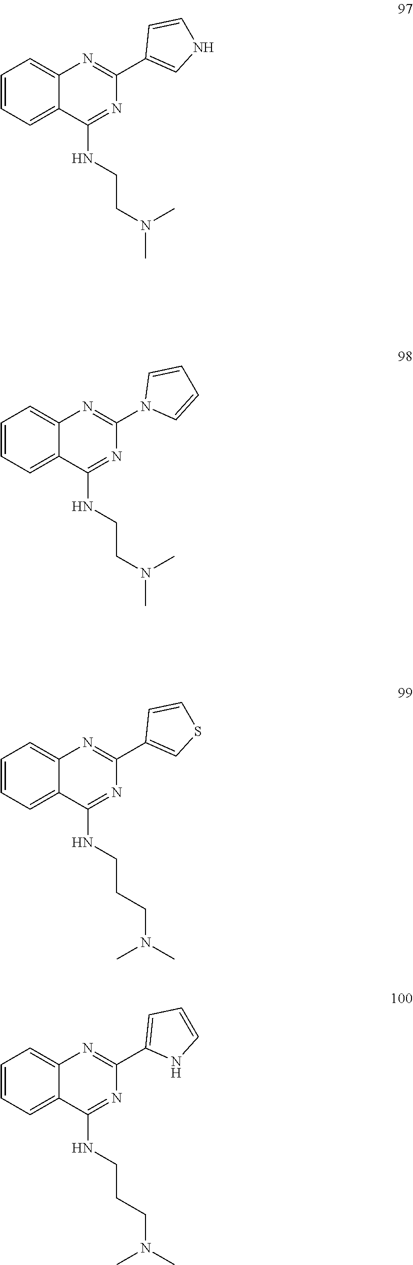 [SMILES: CN(C)CCNc1nc(-c2cc[nH]c2)nc2ccccc12 ; CN(C)CCNc1nc(-n2cccc2)nc2ccccc12 ; CN(C)CCCNc1nc(-c2ccsc2)nc2ccccc12 ; CN(C)CCCNc1nc(-c2ccc[nH]2)nc2ccccc12]

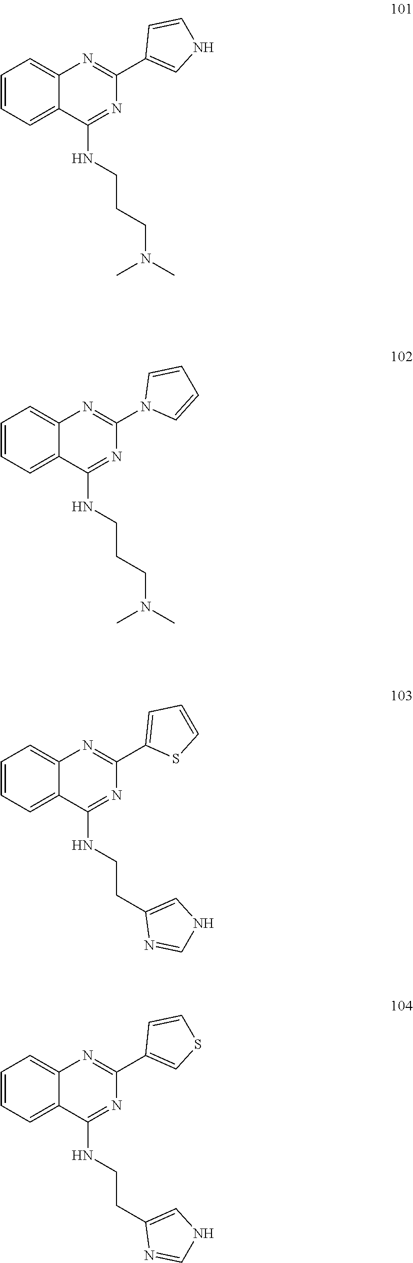 [SMILES: CN(C)CCCNc1nc(-c2cc[nH]c2)nc2ccccc12 ; CN(C)CCCNc1nc(-n2cccc2)nc2ccccc12 ; c1csc(-c2nc(NCCc3c[nH]cn3)c3ccccc3n2)c1 ; c1ccc2c(NCCc3c[nH]cn3)nc(-c3ccsc3)nc2c1]

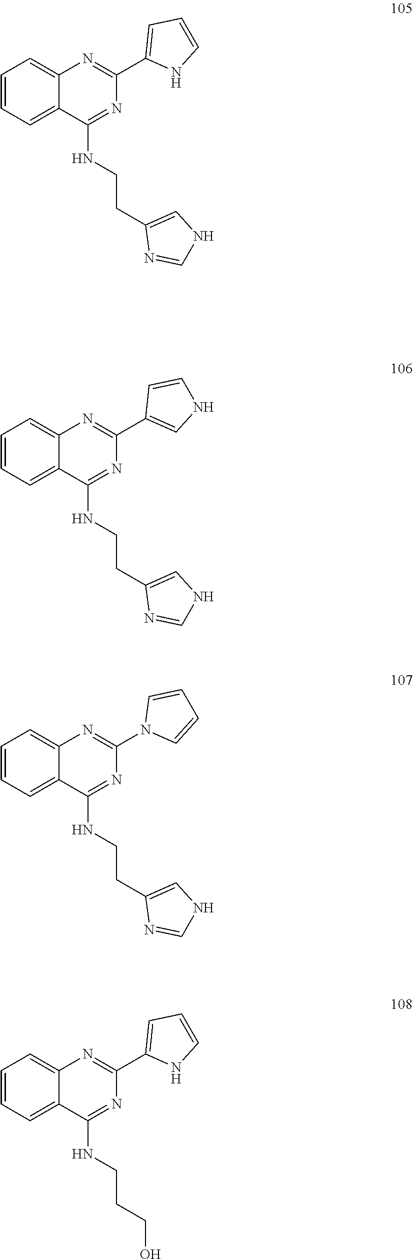 [SMILES: c1c[nH]c(-c2nc(NCCc3c[nH]cn3)c3ccccc3n2)c1 ; c1ccc2c(NCCc3c[nH]cn3)nc(-c3cc[nH]c3)nc2c1 ; c1ccc2c(NCCc3c[nH]cn3)nc(-n3cccc3)nc2c1 ; OCCCNc1nc(-c2ccc[nH]2)nc2ccccc12]

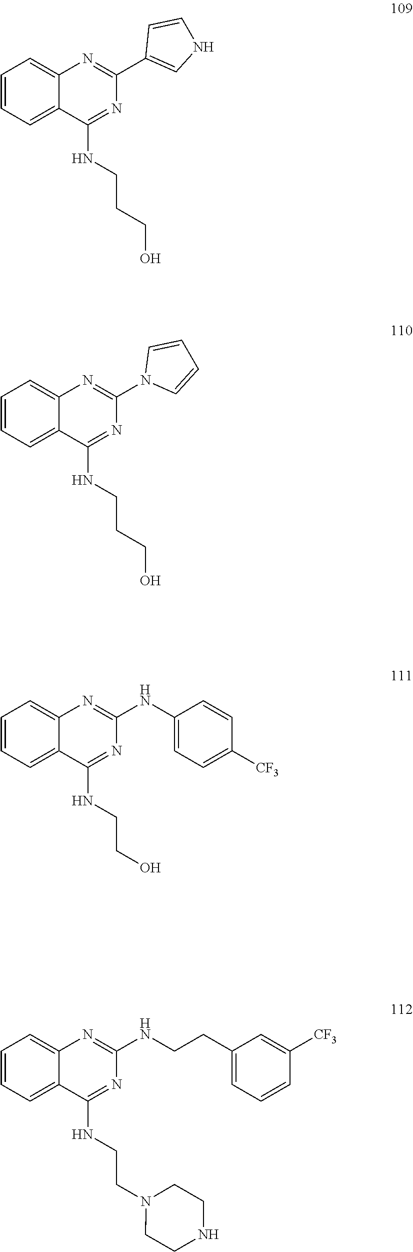 [SMILES: OCCCNc1nc(-c2cc[nH]c2)nc2ccccc12 ; OCCCNc1nc(-n2cccc2)nc2ccccc12 ; OCCNc1nc(Nc2ccc(C(F)(F)F)cc2)nc2ccccc12 ; FC(F)(F)c1cccc(CCNc2nc(NCCN3CCNCC3)c3ccccc3n2)c1]

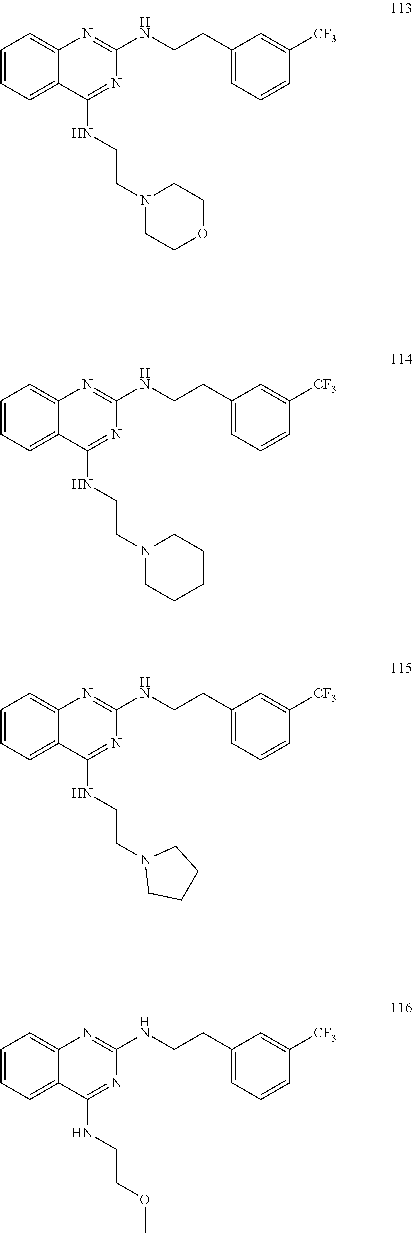 [SMILES: FC(F)(F)c1cccc(CCNc2nc(NCCN3CCOCC3)c3ccccc3n2)c1 ; FC(F)(F)c1cccc(CCNc2nc(NCCN3CCCCC3)c3ccccc3n2)c1 ; FC(F)(F)c1cccc(CCNc2nc(NCCN3CCCC3)c3ccccc3n2)c1 ; COCCNc1nc(NCCc2cccc(C(F)(F)F)c2)nc2ccccc12]

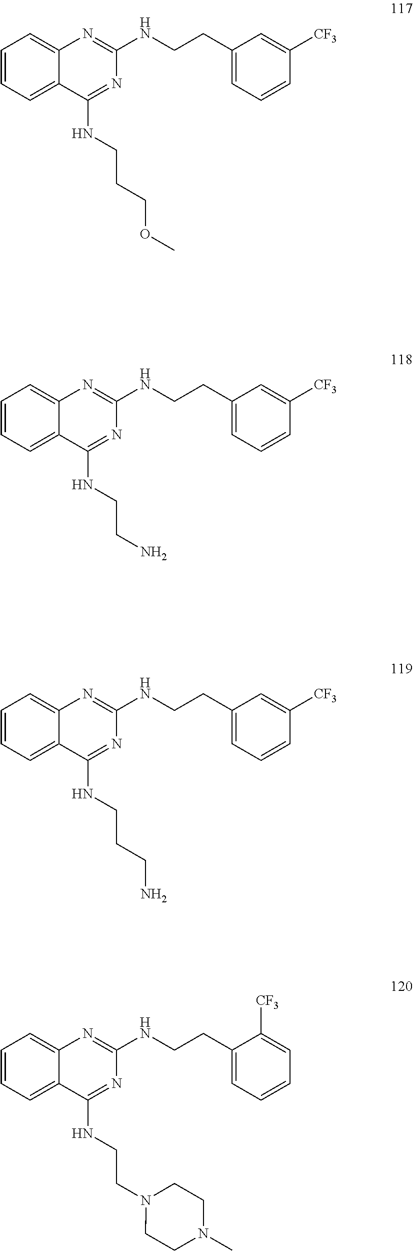 [SMILES: COCCCNc1nc(NCCc2cccc(C(F)(F)F)c2)nc2ccccc12 ; NCCNc1nc(NCCc2cccc(C(F)(F)F)c2)nc2ccccc12 ; NCCCNc1nc(NCCc2cccc(C(F)(F)F)c2)nc2ccccc12 ; CN1CCN(CCNc2nc(NCCc3ccccc3C(F)(F)F)nc3ccccc23)CC1]

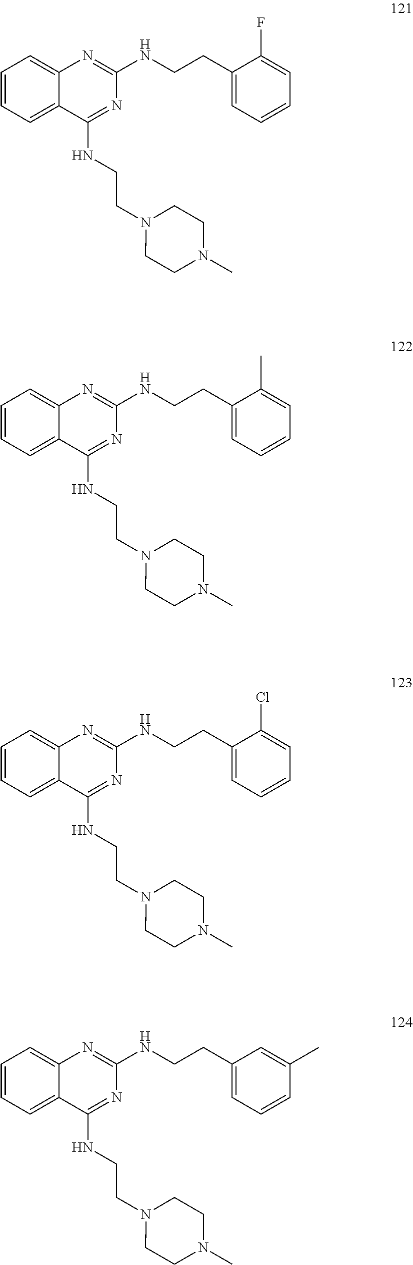 [SMILES: CN1CCN(CCNc2nc(NCCc3ccccc3F)nc3ccccc23)CC1 ; Cc1ccccc1CCNc1nc(NCCN2CCN(C)CC2)c2ccccc2n1 ; CN1CCN(CCNc2nc(NCCc3ccccc3Cl)nc3ccccc23)CC1 ; Cc1cccc(CCNc2nc(NCCN3CCN(C)CC3)c3ccccc3n2)c1]

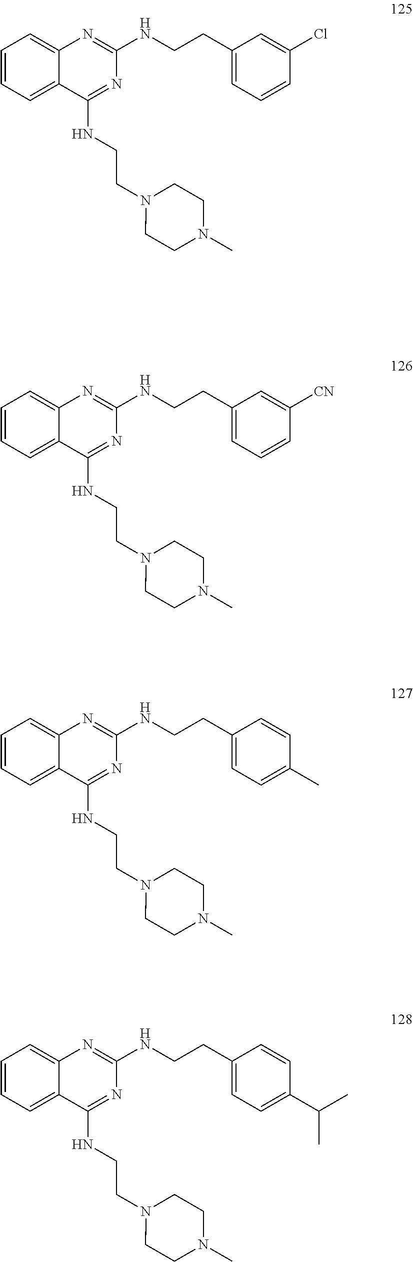 [SMILES: CN1CCN(CCNc2nc(NCCc3cccc(Cl)c3)nc3ccccc23)CC1 ; CN1CCN(CCNc2nc(NCCc3cccc(C#N)c3)nc3ccccc23)CC1 ; Cc1ccc(CCNc2nc(NCCN3CCN(C)CC3)c3ccccc3n2)cc1 ; CC(C)c1ccc(CCNc2nc(NCCN3CCN(C)CC3)c3ccccc3n2)cc1]

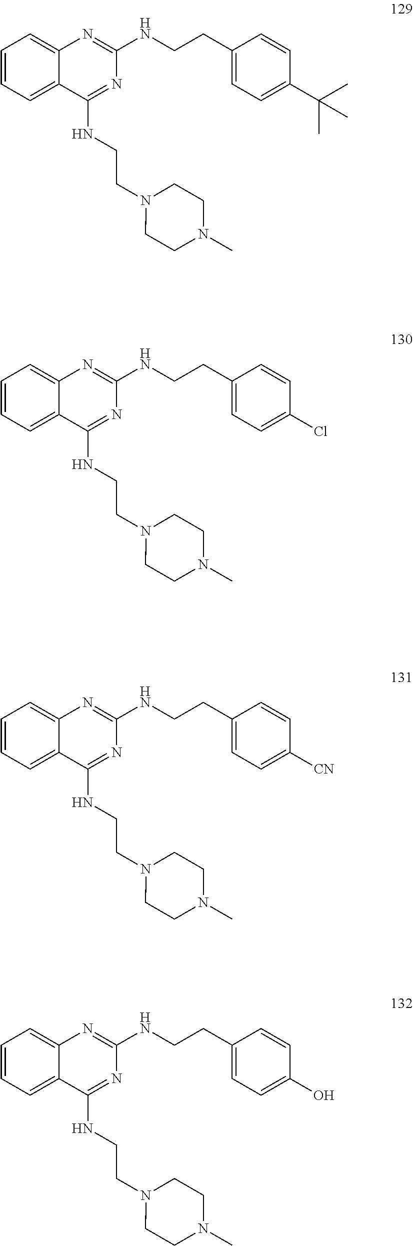 [SMILES: CN1CCN(CCNc2nc(NCCc3ccc(C(C)(C)C)cc3)nc3ccccc23)CC1 ; CN1CCN(CCNc2nc(NCCc3ccc(Cl)cc3)nc3ccccc23)CC1 ; CN1CCN(CCNc2nc(NCCc3ccc(C#N)cc3)nc3ccccc23)CC1 ; CN1CCN(CCNc2nc(NCCc3ccc(O)cc3)nc3ccccc23)CC1]

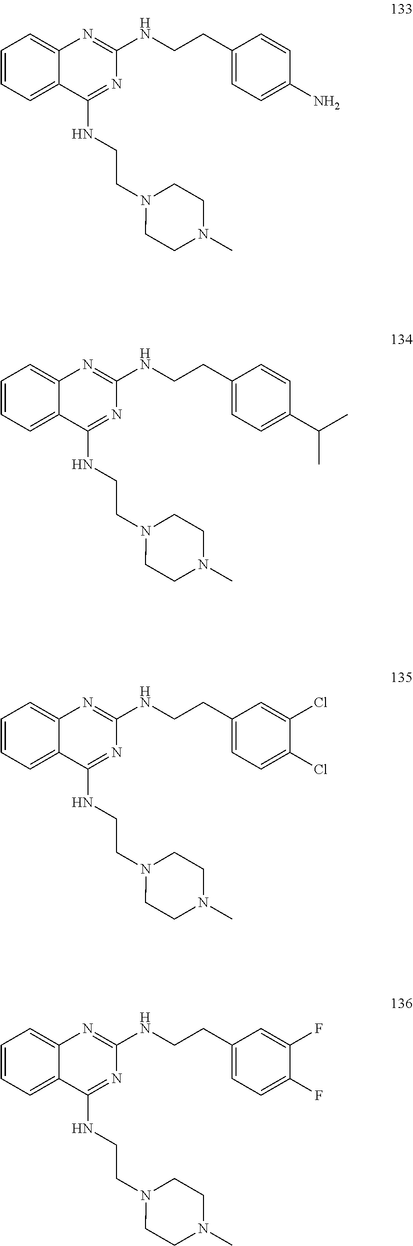 [SMILES: CN1CCN(CCNc2nc(NCCc3ccc(N)cc3)nc3ccccc23)CC1 ; CC(C)c1ccc(CCNc2nc(NCCN3CCN(C)CC3)c3ccccc3n2)cc1 ; CN1CCN(CCNc2nc(NCCc3ccc(Cl)c(Cl)c3)nc3ccccc23)CC1 ; CN1CCN(CCNc2nc(NCCc3ccc(F)c(F)c3)nc3ccccc23)CC1]

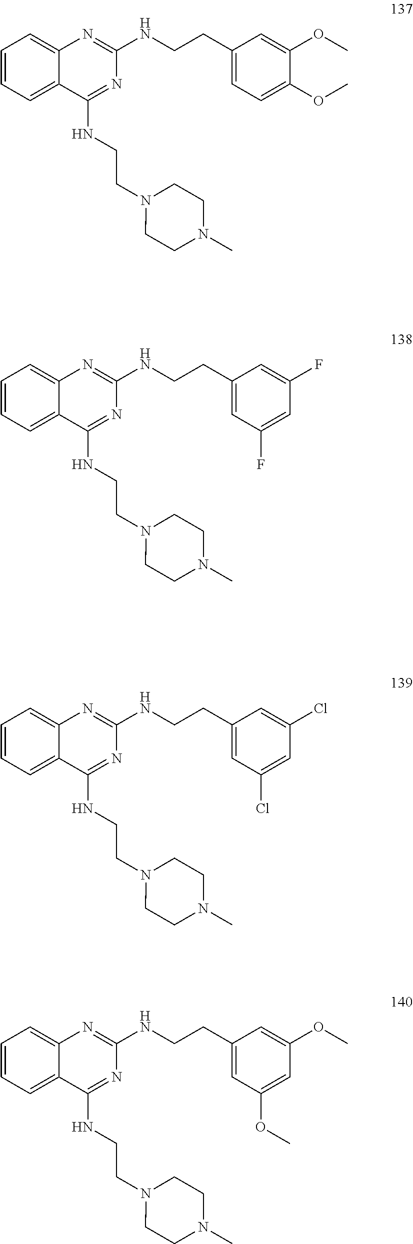 [SMILES: COc1ccc(CCNc2nc(NCCN3CCN(C)CC3)c3ccccc3n2)cc1OC ; CN1CCN(CCNc2nc(NCCc3cc(F)cc(F)c3)nc3ccccc23)CC1 ; CN1CCN(CCNc2nc(NCCc3cc(Cl)cc(Cl)c3)nc3ccccc23)CC1 ; COc1cc(CCNc2nc(NCCN3CCN(C)CC3)c3ccccc3n2)cc(OC)c1]

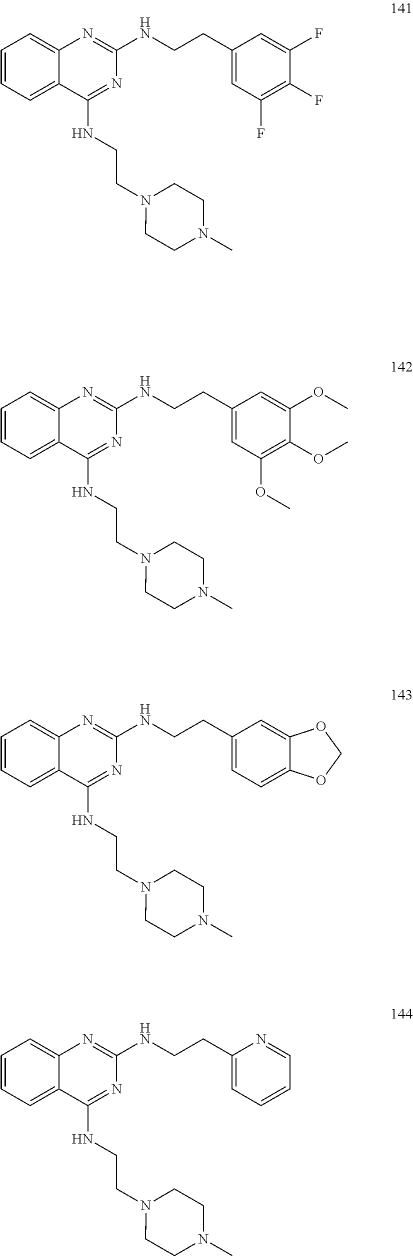 [SMILES: CN1CCN(CCNc2nc(NCCc3cc(F)c(F)c(F)c3)nc3ccccc23)CC1 ; COc1cc(CCNc2nc(NCCN3CCN(C)CC3)c3ccccc3n2)cc(OC)c1OC ; CN1CCN(CCNc2nc(NCCc3ccc4c(c3)OCO4)nc3ccccc23)CC1 ; CN1CCN(CCNc2nc(NCCc3ccccn3)nc3ccccc23)CC1]

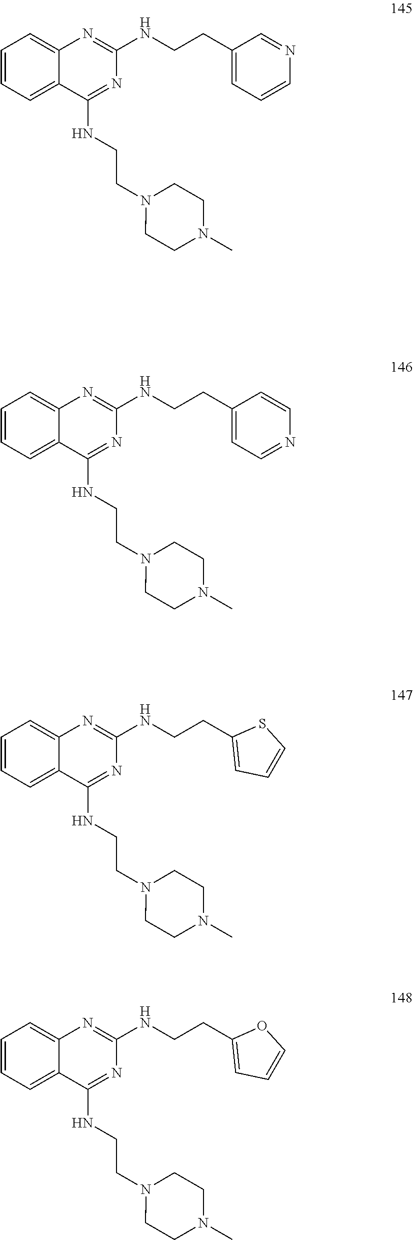 [SMILES: CN1CCN(CCNc2nc(NCCc3cccnc3)nc3ccccc23)CC1 ; CN1CCN(CCNc2nc(NCCc3ccncc3)nc3ccccc23)CC1 ; CN1CCN(CCNc2nc(NCCc3cccs3)nc3ccccc23)CC1 ; CN1CCN(CCNc2nc(NCCc3ccco3)nc3ccccc23)CC1]

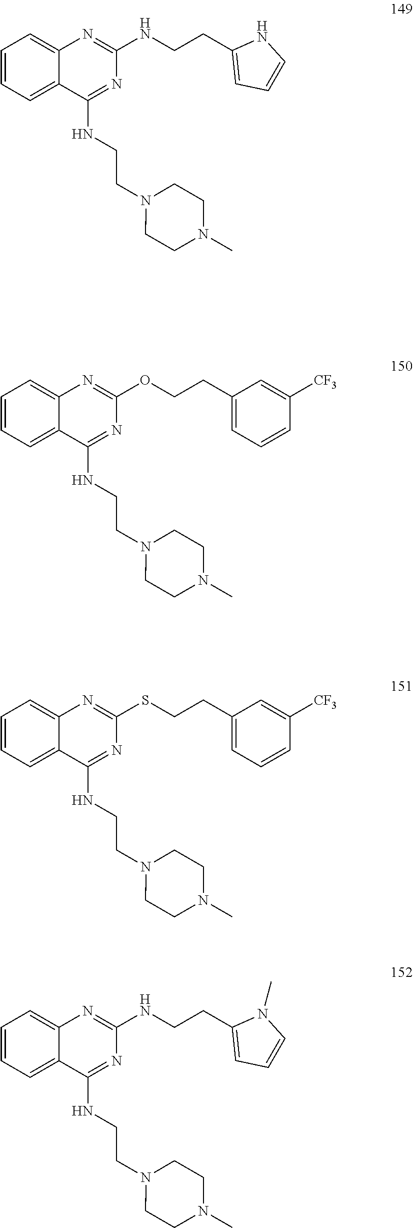 [SMILES: CN1CCN(CCNc2nc(NCCc3ccc[nH]3)nc3ccccc23)CC1 ; CN1CCN(CCNc2nc(OCCc3cccc(C(F)(F)F)c3)nc3ccccc23)CC1 ; CN1CCN(CCNc2nc(SCCc3cccc(C(F)(F)F)c3)nc3ccccc23)CC1 ; CN1CCN(CCNc2nc(NCCc3cccn3C)nc3ccccc23)CC1]

80. A pharmaceutical composition comprising a compound of formula (I) according to claim 1, together with a pharmaceutically acceptable carrier, diluent or excipient.

81.-82. (canceled)

83. A method for the treatment of cancer in a subject in need thereof, the method comprising administration to the subject of a therapeutically effective amount of a compound of formula (I) according to claim 1.

84.-86. (canceled)

87. A method for reducing incidences of, or risk of, cancer recurrence in a subject deemed to be at risk of cancer recurrence, the method comprising administration to the subject of an effective amount of a compound of formula (I) according to claim 1.

88. A method for the treatment of cancer in a subject in need thereof, the method comprising administration to the subject of a therapeutically effective amount of a compound of the following formula (I) 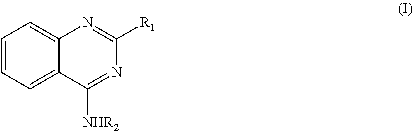
or a pharmaceutically acceptable salt, hydrate, derivative, solvate or prodrug thereof, wherein:
R₁ is selected from the group consisting of:
(i) a heteroaryl group having between 5 and 14 ring atoms in which one or more of the ring atoms are selected from nitrogen, sulfur and oxygen, a heterocyclyl group having between 5 and 14 ring atoms in which one or more of the ring atoms are selected from nitrogen and oxygen,
(ii)
(iii) 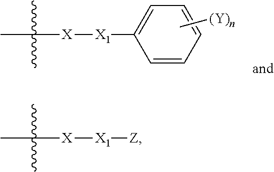
wherein X is O, S or NH;
X₁ is absent or is a straight-chain or branched-chain alkanediyl group having between 1 and 6 carbon atoms;
Y is selected from the group consisting of: CN, NR₅R₆, OH, C₁-C₆alkoxy, halo, CF₃ and C₁-C₆ alkyl;
Z is selected from the group consisting of:a heteroaryl group having 5 or 6 ring atoms in which one or more of the ring atoms are nitrogen, oxygen or sulfur, and (methylenedioxy)phenyl, and wherein the heteroaryl group is optionally substituted with a methyl group;
n is 0, 1, 2 or 3;
R₅ and R₆ are independently selected from the group consisting of: H and C₁-C₆ alkyl;
R₂ is selected from the group consisting of:
(i) T-heteroaryl, wherein the heteroaryl group has 5 ring atoms in which one or more of the ring atoms are nitrogen;
(ii) T-OH
(iii) T-OCH₃
(iv) T-NH₂ and,
(v) 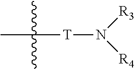
wherein T is a straight-chain or branched-chain alkanediyl group having between 1 and 10 carbon atoms;
R₃ and R₄ are independently selected from the group consisting of: H and C₁-C₆ alkyl, or together with the nitrogen to which they are attached form a saturated 5- or 6-membered ring having 0, 1 or 2 additional nitrogen atoms or 1 or 2 oxygen atoms, wherein the ring is optionally substituted with a C₁-C₆ alkyl group, with the proviso that the following compound is disclaimed:

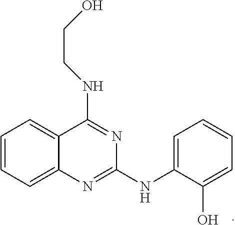 [SMILES: OCCNc1nc(Nc2ccccc2O)nc2ccccc12]

89.-90. (canceled)
